(19) United States
(12) Patent Application Publication
(10) Pub. No.: US 2011/0244496 A1
(43) Pub. Date: Oct. 6, 2011

(54) HIV REVERSE TRANSCRIPTASE COMPOSITIONS AND METHODS

(75) Inventors: Edward V. Arnold, Belle Mead, NJ (US); Joseph Bauman, Somerset, NJ (US); Kalyan Das, Edison, NJ (US)

(73) Assignee: RUTGERS, THE STATE UNIVERSITY, New Brunswick, NJ (US)

(21) Appl. No.: 12/921,390

(22) PCT Filed: Mar. 6, 2008
(86) PCT No.: PCT/US08/56110

§ 371 Date: Jun. 16, 2011

Related U.S. Application Data
(60) Provisional application No. 60/905,168, filed on Mar. 6, 2007.

(51) Int. Cl. C12N 9/12; C12N 15/54; C12N 15/63; C12N 1/00; C12N 5/10; C12Q 1/48

(52) U.S. Cl. 435/15; 536/23.2; 435/320.1; 435/194; 435/243; 435/325; 435/419

(57) ABSTRACT

The present invention provides engineered novel variants of human immunodeficiency virus reverse transcriptase (HIV-RT) capable of being expressed in large quantity and that with polymerase and RNase H activity in a form that facilitates crystallization and high resolution structure resolution following X-ray diffraction. The present invention facilitates high resolution determination of RT in complexes with RT drugs and RT inhibitors, and provides methods for systematic generation of variants and for structure based identification and design of novel RT inhibitors.

This PCT application claims priority to U.S. Provisional Application Ser. No. 60/905,168 filed Mar. 6, 2007, which is incorporated by reference in its entirety.

This invention was made with the support of the National Institutes of Health/NAID Grant Nos.: NIH-NIAID R37 AI027690R01 (Feb. 1, 1998-01/31/09) and NIH-NIGMS P01 GM066671 (Aug. 1, 2002-Aug. 31, 2007). The United States Government may have certain rights to this invention.

Throughout this application, various publications are referenced by name or by number. Full citations for these publications may be found listed at the end of the specification and preceding the claims. The disclosures of these publications in their entireties are hereby incorporated by reference into this application in order to more fully describe the state of the art. A Sequence Listing is also provided.

### FIELD OF ME INVENTION

The present invention relates to engineered novel variants of human immunodeficiency virus (HIV) reverse transcriptase (RT), a primary target for anti-HIV agents. The present invention provides novel HIV-RT constructs capable of being expressed in large quantity and that provide polymerase and RNase H activity. The present invention further provides RT in a form that facilitates crystallization and high resolution structure resolution upon X-ray diffraction (better than 2.0 Å). Thus, the present invention facilitates high resolution determination of RT in complexes with RT drugs and RT inhibitors, thereby facilitating structure-based design of new RT inhibitors.

### BACKGROUND OF THE INVENTION

The production of effective and safe treatment against harmful viruses and other pathogens such as HIV continues to be a difficult endeavor. To date, even the most successful treatments are subject to viral breakthrough by drug-resistant virus strains.

HIV-1 reverse transcriptase (HIV-RT) is responsible for generating double-stranded DNA from the single stranded RNA packaged in the HIV-1 virus. Twelve of the 25 approved anti-AIDS drugs target RT (hivinsite.ucsf.edu, 2007). The two classes of approved RT inhibitors are nucleoside/nucleotide RT inhibitors (NRTIs) and non-nucleoside RT inhibitors (NNRTIs). A high rate of viral turnover combined with lack of efficient proofreading activities in both the RT and human RNA polymerase II involved in HIV-1 replication results in a pool of mutant viruses (Telesnitsky and Goff, 1997). The ability to mutate rapidly enables HIV-1 to develop resistance to anti-AIDS drugs, sometimes within days to a few months of treatment (Larder and Kemp, 1987). New anti-AIDS drugs must overcome the resistance that limits the efficacy of existing drugs. To overcome some of these complications, it is highly desirable to develop methodologies and reagents that will direct antiviral treatments that are stable and resistant to viral breakthrough.

Protein crystal engineering through mutagenesis has been used to determine crystal structures of previously intractable drug/HIV-1 RT complexes. Structures play an important role in designing inhibitors of RT. High-resolution structures can be critical in designing RT inhibitors but RT complexes have usually been structurally determined at ˜3.0 Å.

RT is a heterodimer consisting of p66 and p51 subunits with mass 66 and 51 kDa, respectively. The p51 subunit is formed when the RNase H domain of p66 is proteolytically removed at residue 440 by HIV protease. RT crystallizes with different space groups, unit cells, and X-ray diffraction resolution depending on the complex (e.g. +/−nucleic acid, +/−NNRTI, etc.) and the RT construct. Three different RT constructs, varying in termini and strain sequence, have been used for RT/NNRTI complex crystallization, each crystallizing with characteristic space group symmetry: P2₁2₁2₁, (Ren et al., 1995); C2, (Kohlstaedt et al. 1992; Ding et al., 1995); and C222₁, (Hogberg et al., 2001).

NNRTIs are a diverse set of inhibitors first discovered in Janssen Pharmaceuticals in 1987 (Pauwels et al., 1990). Crystal structures have been instrumental in the development of NNRTIs. Some of the major discoveries from structural studies include: 1. All NNRTIs bind in the same NNRTI binding pocket (NNIBP); 2. Different classes of NNRTIs have distinct modes of binding including the mechanism of entrance into the NNIBP; 3. The NNIBP exists in a closed form when an NNRTI is not bound; and 4. The NNIBP is elastic, and its confirmation depends on the NNRTI bound (for review see Das et al., 2004 and 2005). The elastic nature of the NNIBP poses a challenge for computational modeling and molecular dynamic simulations as both the target and ligand are flexible.

NNRTIs do not affect binding of RT to the nucleic acid substrate or to dNTPs (Rittinger et al., 1995; Spence et al., 1995). Recently, evidence for the mechanism of NNRTI inhibition was shown through two crystal structures produced in the presence of ATP and Mn⁺² with and without the NNRTI HBY 097. The structure with NNRTI bound contains an ATP coordinated by two Mn⁺² at the polymerase active site. The coordination is not present in the NNRTI-bound crystal form. NNRTI may restrict the flexibility of the YMDD active site loop and thereby prevent the catalytic aspartate residues (185 and 186) from binding the two Mn⁺² (Das et al., 1998 and 2007).

TMC278, belonging to the diarylpyrimidine (DAPY) class of NNRTIs, is currently in advanced Phase-II clinical trials in the USA and Europe. It was developed by a multidisciplinary effort involving medicinal chemists, virologists, crystallographers, molecular modelers, toxicologists, analytical chemists, and pharmacologists (Janssen et al., 2005). Short-term results from a Phase-H clinical trial were recently published showing the efficacy of TMC278 in HIV-1 infected patients (Goebel et al., 2006). Antiretroviral naïve patients were given a once daily dosage of 25, 50, 100, or 150 mg of TMC278 for seven days. Their HIV-1 RNA viral loads were measured before the initial dosage and on day 8; results showed an average decrease of 1.199 log₁₀ copies/ml versus an increase of 0.002 log₁₀ copies/ml in the placebo group. The side effects were found to be less than the placebo group with headaches reported in 14% of the patients given TMC278 versus 18% in the placebo group. Bioavailability was also shown to be excellent with plasma concentrations of TMC278 not below the target concentration of 13.5 ng/ml at any of the time points tested.

Structural studies have been utilized in determining the mechanism of NNRTI-resistance mutations. Tyr181Cys and Tyr188Cys directly alter the binding ability of the NNRTI to the NNIBP by elimination of π-π interactions between the protein and ligand. Leu101Ile and Gly103Ala cause steric hindrance for the NNRTI by altering the shape of the NNIBP. Lys103Asn uses an unexpected mechanism for resistance; it stabilizes the closed form of the NNIBP by coordinating a sodium atom with residues Lys101 and Tyr188. The stabilization of the NNIBP by the Lys103Asn mutation creates an additional energetic penalty for entry that the NNRTI must compensate for by additional interactions (Hsiou et al., 2001; Das et al., 2007).

Indeed, structural studies were instrumental in developing the DAPY class of NNRTIs, including TMC278/rilpivirine and TMC125/etravirine, which inhibit wild-type and drug-resistant HIV-1 viruses (Janssen et al 2005). The DAPY NNRTIs have strategic flexibility, allowing them to inhibit NNRTI resistant RT (Das et al., 2004; Das et al., 2008). In early attempts to crystallize RT/TMC278 complex, the crystals failed to diffract beyond 6 Å resolution. The conformational flexibility of TMC278 potentially introduced heterogeneity in the arrangement of RT molecules in the crystal lattice (Das et al. 2005), which may have been responsible for low resolution diffraction obtained in many trials over five years. Ineed, during the development of the DAPY class of compounds, structural studies using X-ray crystallography were used to determine the modes of binding and effects of resistance mutations on the potency of the inhibitor candidates. Numerous crystal structures, with and without resistance mutations, showed the existence of an NNRTI-binding pocket (NNIBP), which is not present in crystal structures where RT is not complexed with an NNRTI (Ding et al., 1995a; Ding et al., 1995b; Esnouf et al., 1995; Ren et al., 1995a; Ren et al., 1995b; Das et al., 1996; Hopkins et al., 1996; Esnouf et al., 1997; Hsiou et al., 1998; Ren et al., 1998; Hopkins et al., 1999; Ren et al., 1999; Hogberg et al., 2000; Ren et al., 2000a; Ren et al., 2000b; Chan et al., 2001; Hsiou et al., 2001; Ren et al., 2001; Chamberlain et al., 2002; Lindberg et al., 2002; Das et al., 2004; Hopkins et al., 2004; Pata et al., 2004; Ren et al., 2004; and Das et al., 2007). The formation of the NNIBP only in the presence of NNRTIs and the binding of a chemically diverse set of inhibitors to the same pocket is one of the major results of crystallographic studies of RT complexed with NNRTIs. The flexible nature of the NNIBP (depending on the arrangement of the NNRTI bound) limits the usefulness of non-crystallographic structural studies (i.e., molecular dynamic modeling) due to the complexity of studying both a flexible ligand and target. Understanding the mechanism of binding and resistance to early DAPYs and other NNRTIs, by structural studies, led to the development of the current inhibitors including TMC125-etravirine and TMC278-rilpivirine (Das et al., 2004 and 2005). TMC278 is a potent inhibitor of NNRTI-resistant HIV-1 strains including the Leu100Ile/Lys103Asn and Lys103Asn/Tyr181Cys double mutants, which are resistant to all approved NNRTIs. The strategic flexibility of TMC278 may have been responsible for no diffraction quality crystals being obtained in five years of trials.

According to the approach of the present invention, restriction of RT conformations in the crystal lattice through protein engineering was employed to improve diffraction quality.

The present invention therefore provides a systematic protein crystal engineering approach to solve the problem of the prior art and to obtain improved crystal structures of the RT/TMC278 complex. There are three fundamental types of protein engineering approaches for crystallography: 1) alterations affecting the suitability of the protein for biochemical study including mutagenesis and the addition of tags for expression, solubility, and purification; 2) engineering to increase the conformational homogeneity of the protein sample; and 3) modification of the protein to directly alter interactions at crystal contact interfaces (for reviews Dale et al., 2003 and Derewenda, 2004). Examples of engineering to increase the homogeneity of the sample include addition and subsequent removal of purification tags; deletions of disordered regions including termini, loops, and domains; and replacement of highly entropic residues (e.g., lysines and glutamic acids) by the surface entropy reduction method. Rational alterations of the protein for crystallization include substitution of residues known to be required for crystallization of a homologous protein, systematic or random alteration of surface residues to create a library of potentially crystallizable proteins, and alteration of known crystal contacts to create potentially new crystal forms.

The present invention, for the first time, provides the crystal structures of TMC278 with and without the NNRTI-resistance mutations Leu100Ile/Lys103Asn and Lys103Asn/Tyr181Cys. The structure of TMC278 complexed with the provided engineered RT, RT52A, at 1.8 Å resolution, has the highest resolution ever obtained for any HIV-1 RT structure.

According to the present invention, engineered RTs were co-crystallized with TMC278, and screened for quality of X-ray diffraction data. Several iterative rounds of mutagenesis and crystallization with TMC278 were employed to produce a construct that produced improved diffraction with this important drug candidate. One construct, RT52A, provided by the present invention, is a product of multiple iterative rounds of design. RT52A produces crystals within hours to days of crystal drop generation with and without microseeding. High-resolution datasets, some better than 2.0 Å, can now be produced quickly and reproducibly for most of the NNRTIs tested.

Most notably, TMC278 was structurally solved to 1.8 Å resolution; thousands of crystallizations prior to this effort had yielded only 8 Å resolution crystal diffraction. This is compared to previous RT/inhibitor crystals, which in favorable cases formed in days to weeks with microseeding and structural resolution of 25 to 3.0 Å. The previous highest resolution RT structure was 22 Å. The swiftness of crystallization of this new construct allows for high-throughput structure-based design of new NNRTIs. Further protein engineering was carried out to obtain high-resolution structures of unliganded RT and RT/RNase H inhibitor complexes. The improved resolution enables a detailed understanding of drug resistance, designing improved drugs against existing targets in RT, and in finding novel sites for new types of RT inhibitors.

The present invention utilized a co-expression system that facilitates subunit-specific mutagenesis at multiple positions and the addition of a purification tag on the C or N terminus of the subunit of choice for facile purification. In the initial co-expression construct, the p51 subunit consisted of 428 residues and a hexahistidine purification tag at the C terminus (Huang et al., 1998 and Sarafianos et al., 2003). The co-expression construct codes for the p66 Q258C mutant, which is used to produce homogenous nucleic-acid cross-linked samples for X-ray crystallographic studies. This plasmid facilitates expression, purification, and crystallization of multiple RT constructs in parallel.

The present invention utilized two methods of expression/purification methods for RT. RT is a heterodimer consisting of a p66 and p51 subunit. The p51 subunit is identical to p66 with the RNase H domain proteolytically removed at residue 440. According to the first method p66 is expressed in E. coli, and then it is purified using laborious chromatography techniques. A co-purifying E. coli protease cleaves the p66 into a p66:p51 heterodimer, which is then further purified to homogeneity (Clark et al., 1990). This protein, referred to as 1B1, has been extensively used for NNRTI structural studies. The second method uses co-expression of the p66 and p51. In the co-expression construct the p51 subunit terminates at residue 428, and a hexahistidine purification tag is appended at the C terminus (Sarafianos et al., 2004). The co-expression construct codes for a Q258C mutation that has been used in cross-linking experiments to link nucleic acid substrates; however, this construct has not been successfully used with NNRTIs. To produce large numbers of subunit specific mutants and express/purify them in parallel, the co-expression method was used.

### SUMMARY OF THE INVENTION

The present invention provides an isolated nucleic acid molecule encoding a peptide comprising the amino acid sequences of SEQ ID NO:1 and SEQ ID NO:2, representing the p66 and p51 subunits of HIV-RT.

The present invention also provides an isolated nucleic acid molecule encoding at least a portion of the amino acid sequence of human immunodeficiency virus reverse transcriptase (HIV-RT) wherein: (a) the amino-terminus of HIV-RT p66 comprises amino acid residues MVPISP (SEQ ID NO: 4); (b) the nucleic acid molecule encodes alanine at amino acid residue 172 of p66; (c) the nucleic acid molecule encodes alanine at amino acid residue 173 of p66; (d) the nucleic acid molecule encodes serine at amino acid residue 280 of p66; (e) the nucleic acid molecule encodes serine at amino acid residue 280 of p51; (f) the carboxy-terminus of p66 terminates at residue 555; and (g) the carboxy-terminus of HIV-RT p51 terminates at residue 428.

The present invention also provides an isolated nucleic acid or portion thereof wherein the nucleic acid (a) encodes at least a portion of a human immunodeficiency virus (HIV) reverse transcriptase (RT); and (b) is capable of hybridizing under standard hybridization conditions to the provided nucleic acid sequence or complement thereof. According to specific embodiments of this invention, the provided nucleic acid is capable of hybridizing with a nucleic acid or its complement that is capable of encoding SEQ ID NO:1 or SEQ ID NO: 2.

The present invention further provides a method for generating crystallization variants of an HIV-RT-NNRTI complex, comprising the steps of: (a) truncating at least one terminus of HIV-RT; (b) reducing surface lysine acid regions; and (c) mutating at least one amino acid residue, thereby altering lattice contact from the non-mutated residue.

Finally, the present invention provides A method for identifying HIV-RT inhibitor solvent molecules comprising the steps of (a) soaking a small molecule fragment into a crystallization variant generated by the provided method, thereby forming an HIV-RT complex with the molecule; (b) determining three dimensional structure of the complex; and (c) determining HIV-RT enzyme activity.

### BRIEF DESCRIPTION OF THE FIGURES

FIGS. 1A-1C. Molecular cloning of RT. (A) 6.7 kilo-base pair expression vector with unique restriction sites for p51 and p66. (B) Diagram of RT1A and RT52A. (C) Schematic showing binding sites of the 2′-O-methylated primers used in MOE-LIC.

FIGS. 2A-2B. Magnified images of RT crystals. (A) Images of crystals of round three mutant RT in complex with an NNRTI, CL32543. When grid is present, one grid is 0.12 mm on an edge. (B) Images of crystals from round four and five mutagenesis.

FIGS. 3A-3B. Enzymatic activities of engineered RT round four mutants. (A) DNA-dependent DNA polymerase processivity assay using a 5′ end-labeled primer annealed to single-stranded M13mp18 DNA. RT is allowed to bind in the absence of dNTPs. dNTPs and a “cold trap” (poly[rC].oligo[dG]) are added and the reaction incubated at 37°. Presence of the “cold trap” limits the polymerase reaction to one cycle of extension. (B) RNase H activity assay. A 5′ end-labeled RNA is annealed to a DNA primer. The template/primer is incubated with the various RTs for the indicated length of time. An untreated sample is included to show the size of the full-length RNA.

FIGS. 4A-4B. Structure of RT52A with TMC278 at 1.8 Å. (A) Cartoon of RT52A with the p66 domains labeled. The YMDD polymerase active site is labeled in azure while TMC278 is in gray. (B) 2Fo-Fc map of TMC278 in the NNRTI binding pocket. The NNRTI binding pocket residues mutated are shown.

FIG. 5. Cartoon showing RT mutations.

FIG. 6. Contacts in RT crystals. Residues within 4.5 Å of a symmetry related residue are labeled in spheres. The 1B1/NNRTI structure is PDB code 1S9E. Additional regions involved in crystal contacts in the RT52A and RT69A structures are from both the p66 and p51 subunits.

FIGS. 7A-7C. Resolution distribution of RTs. (A) RT52A and RT69A datasets compared to published RT data sets. Number of structures are plotted against resolution. Total unique reflections in 2.8, 2.2, and 1.8 Å datasets are shown. (B) Diagram of tested RTs. Shown are mutants which produced low resolution diffracting crystals or no crystals, mutants producing medium 3-4 Å resolution diffracting crystal, 2-3 Å resolution diffracting crystal producing constructs, and RTs producing crystals which diffracted to better than 2 Å. (C) Plot of inverse resolution of tested mutants, scaled by the one minus the exponent of the inverse of the resolution (1−Exp(1/resolution in Å). Actual resolution is indicated.

FIG. 8. Ramachandran plot and statistics for RT52A/TMC278.

FIGS. 9A-9C. RT52A/TMC278 structure and p66 fingers crystal contacts. (A) Cartoon of RT52A/TMC278 with p66 subdomains labeled. TMC278 is colored in grey and the polymerase active site in cyan. (B) Cartoon viewed from below. (C) From the same viewing angle as B the p66 subunit makes crystal contacts with the thumb subdomain of one symmetry molecule and the RNase H domain of another molecule.

FIGS. 10A-10B. Overlay of engineered RTs and wild-type RT. (A) Ribbon diagram of the RT structures from this study and wild-type RT/R129385 produced in MacPyMol. (B) Alignment with secondary structure overlay. Includes the sequence from the structure of RT/nevirapine complex (PDB code: 1VRT).

FIGS. 11A-11D. TMC278 bound in the wild-type NNIBP. (A) Representation of TMC278 in the NNIBP of RT52A. Amino acids lining the NNBP are labeled. (B) The cyanovinyl and Wing 1 reside inside the hydrophobic core of the NNIBP. (C) Electron density defines the position of TMC278 in the NNIBP. (D) TMC278 with the cyanovinyl and labeled torsion angle locations.

FIGS. 12A-12C. Structural comparison of L100I and K103N double mutant to the wild-type NNIBP. (A) Omit map defines the position of the inhibitor. (B) Overlay showing the torsional flexibility of TMC278 (wiggling) when bound to the mutant RT. (C) A perpendicular view showing the spatial movement of TMC278 (jiggling).

FIGS. 13A-13B. Structural comparison of K103N and Y181C double mutant to the wild-type NNIBP. (A) Omit map defines the position of the inhibitor. (B) Overlay showing the adjustment of Y183 to compensate for the Y181C mutation.

FIG. 14. Published molecular structures of TMC120 inhibitor used in RT engineering study.

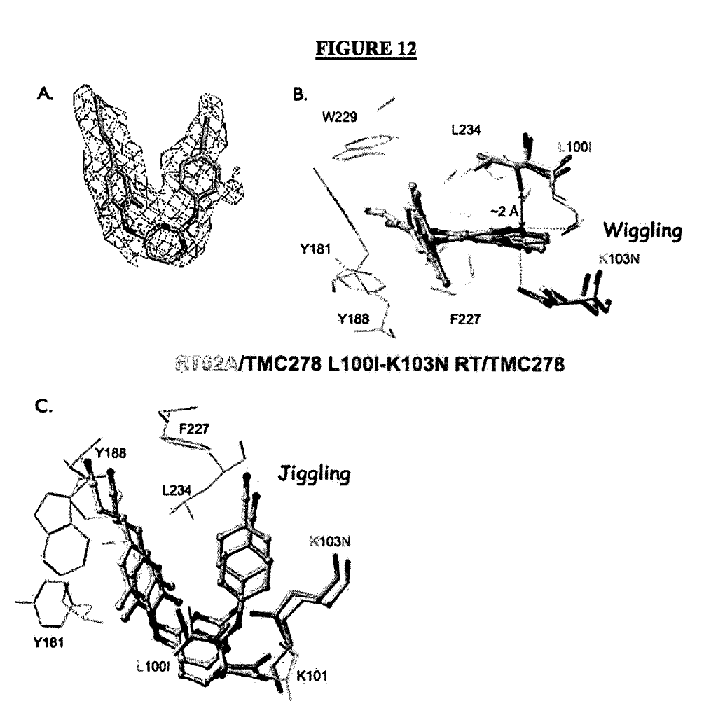

FIG. 15. Published molecular structures of TMC125 inhibitor used in RT engineering study.

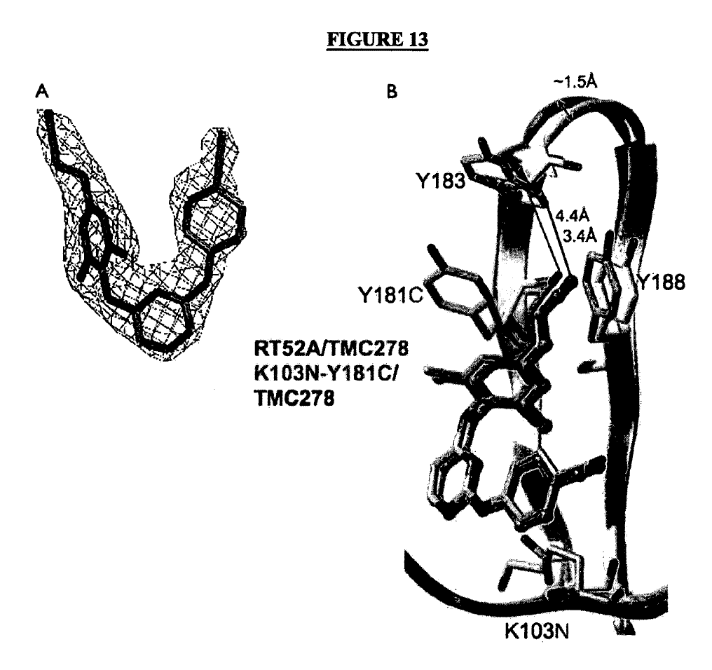

FIG. 16. Published molecular structures of TMC278 inhibitor used in RT engineering study.

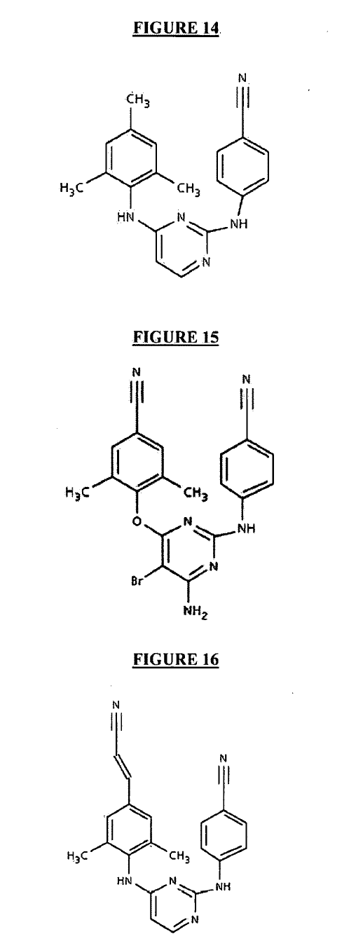

FIG. 17. Iterative approach to crystal engineering of the present invention.

FIGS. 18A-18E. Mutagenesis of RT of the present invention. (A) Schematic showing the binding sites (arrows) of the 2′-O-methylated primers used in MOE-LIC.

(B) Annealing of the primer terminated insert and vector; 2′-O-methyl nucleotides are indicated with Me. (C) Cartoon of RT color-coded by the p66 subdomains. All mutations made in this study are indicated as spheres. The beneficial mutations are labeled. (D) Flowchart of mutants coded by crystal X-ray diffraction resolution. Stars mark mutants with improved resolution unliganded (E) Diagram of RT1A, RT52A, and RT69A.

FIGS. 19A-19C. Crystal Structure of RT52A with TMC278 at 1.8 Å resolution. (A) Simulated annealed Fo-Fc omit map (3□ contours) for TMC278. (B) Typical 1B1-RT arrangement in a crystal unit cell (pdb code: 1S9E). (C) A relatively compact packing of RT52A molecules in the crystal lattice of RT52A/TMC278 complex.

FIG. 20. Comparison of unit cell and X-ray diffraction resolution of mutants. Plot of unit cell (Matthews Coefficient) and X-ray diffraction resolution (Å) of the mutants that produced crystals that diffracted X-rays to better than 4 Å resolution. The legend table indicates the mutations and the template for each of the mutants. RT69A and RT97A are plotted based on crystals complexed with RNHIs bound, all others with NNRTIs.

FIGS. 21A-21C. (A) Overall structure of the wild-type HIV-1 RT/TMC278 complex determined at 1.8 Å resolution. (B) The position and conformation of TMC278 were defined by the difference (|Fo|−|Fc|) electron density calculated at 1.8 Å resolution (3.5□ contours). (C) Chemical structure of TMC278. The □ angles define the torsional flexibility of TMC278.

FIGS. 22A-22B. (A) Interactions of TMC278 with NNRTI-binding pocket residues. (B) The molecular surface defines the hydrophobic tunnel that accommodates the cyanovinyl group of TMC278.

FIG. 23. Superposition of K103N/Y181C mutant RT/TMC278 complex on the wild-type RT/TMC278 complex. The YMDD motif in the mutant structure is repositioned closer to TMC278, this leads to an important interaction between the cyanovinyl group and the highly conserved Y183 residue. Despite the rearrangements in the inhibitor position and conformation and the binding-pocket residues, the extents of the inhibitor-protein interactions remain almost unchanged.

FIGS. 24A-24B. Comparison of L100I/K103N mutant RT/TMC278 structure with the wild-type RT/TMC278 structures reveals (A) wiggling and (B) jiggling of TMC278.

### DETAILED DESCRIPTION

The present invention provides an isolated nucleic acid molecule encoding a peptide comprising the amino acid sequence of SEQ ID NO:1. SEQ ID NO: 1 encodes the p66 subunit, of HIV-RT. According to one embodiment of the invention, the provided nucleic acid molecule further comprising SEQ ID NO: 2. SEQ ID NO: 2 encodes the p51 subunit of HIV-RT. According a preferred embodiment, the provided nucleic acid molecule is capable of expressing the p66/p51 heterodimer. According to a most preferred embodiment, the provided nucleic acid encodes the p66 subunit and the p51 subunit in different open reading frames. According to another preferred embodiment, separate promoters control expression of the p66 and p51 subunits. According to another embodiment the invention provides an isolated nucleic acid molecule encoding a peptide comprising the amino acid sequence of SEQ ID NO:2.

The present invention also provides an isolated nucleic acid molecule encoding at least a portion of the amino acid sequence of human immunodeficiency virus reverse transcriptase (HIV-RT) wherein at least one terminal end of the protein is truncated. According to a preferred embodiment of this invention, truncation of an HIV-RT terminus facilitates resolution of three dimensional crystal structure. It is specifically contemplated by the invention that any combination of HIV-RT termini may be truncated so long as they facilitate resolution of the three dimensional crystal structure of the protein. According to a preferred embodiment of the invention, the HIV-RT is complexed with a NNRTI ligand. NNRTI ligands are well known in the art and include the DAPY compounds. According to a preferred embodiment of this invention the Still further, the present invention provides HIV-RT in complex with TMC278. According to an alternative embodiment, the invention provides HIV-RT in complex with TMC125.

The present invention further provides an isolated nucleic acid molecule encoding at least a portion of the amino acid sequence of human immunodeficiency virus reverse transcriptase (HIV-RT) wherein: (a) the amino-terminus of HIV-RT p66 comprises amino acid residues MVPISP (SEQ ID NO: 4); (b) the nucleic acid molecule encodes alanine at amino acid residue 172 of p66; (c) the nucleic acid molecule encodes alanine at amino acid residue 173 of p66; (d) the nucleic acid molecule encodes serine at amino acid residue 280 of p66; (e) the nucleic acid molecule encodes serine at amino acid residue 280 of p51; (f) the carboxy-terminus of p66 terminates at residue 555; and (g) the carboxy-terminus of HIV-RT p51 terminates at residue 428. According to one embodiment, the amino-terminus of p51 further comprises a human rhinovirus subtype 14 3C (HRV-14 3C) protease cleavage site, wherein the HRV-14 3C protease cleavage site is situated between a hexaHIS purification tag and the p51 coding sequence, thereby facilitating generation of a post-protease amino-terminus of gPISP upon exposure to HRV-14 3C protease under standard conditions for HRV-14 3C protease activity. According to a preferred embodiment, the isolated nucleic acid molecule comprises the nucleic acid sequence of SEQ ID NO 3. According to another embodiment, the invention provides a recombinant vector comprising the nucleic acid molecule of SEQ ID NO: 3. According to another embodiment, the present invention provides a nucleic acid molecule that encodes HIV-RT p66 and the amino-terminus of p66 begins with the amino acid residues MVPISP (SEQ ID NO: 121). According to yet another embodiment, the present invention provides a nucleic acid molecule that encodes at least a portion of the amino acid sequence of human immunodeficiency virus reverse transcriptase (HIV-RT), wherein the nucleic acid molecule encodes alanine at amino acid residue 172 of p66. According to yet another embodiment, the present invention provides a nucleic acid molecule that encodes HIV RT p66 and wherein the amino terminus of p66 comprises amino acid residues MVPISP (SEQ ID NO: 121). According to still yet another embodiment, the present invention provides a nucleic acid molecule that encodes alanine at amino acid residue 173 of p66. Still according to another embodiment, the present invention provides a nucleic acid molecule that encodes serine at amino acid residue 280 of p66. According to a further embodiment, the present invention provides a nucleic acid molecule that encodes serine at amino acid residue 280 of p51. Further still, according to another embodiment, the present invention provides a nucleic acid molecule that encodes HIV RT p66 and wherein the carboxy-terminus of p66 terminates at residue 555. It is understood that the termination residue for the naturally occurring protein is 560. Still another embodiment provides that the nucleic acid molecule encodes HIV RT p51 and wherein the amino-terminus of p51 comprises a human rhinovirus subtype 14 3C protease (HRV-14 3C) cleavage site. According to a preferred embodiment of this invention, the HRV-14 3C protease cleavage site is situated between a hexaHIS purification tag and the p51 coding sequence, thereby facilitating generation of a post-protease amino-terminus of gPISP upon exposure to HR V-14 3C protease under standard conditions for HRV-14 3C protease activity. According to a still further embodiment, the nucleic acid molecule encodes the carboxy-terminus of p51 terminates at residue 428. It is understood that the termination residue for the naturally occurring protein is 440. The present invention also provides the HIV-RT product of the expression of the provided nucleic acid.

The present invention also provides an isolated nucleic acid or portion thereof wherein the nucleic acid (a) encodes at least a portion of a human immunodeficiency virus (HIV) reverse transcriptase (RT); and (b) is capable of hybridizing under standard hybridization conditions to the provided nucleic acid sequence or complement thereof. According to specific embodiments of this invention, the provided nucleic acid is capable of hybridizing with a nucleic acid or its complement that is capable of encoding SEQ ID NO: or SEQ ID NO: 2. It is well understood and specifically contemplated by the present invention that the provided recombinant vector may be in the form of a replicon. According to a preferred embodiment of this invention, the vector is a plasmid. According to yet another embodiment of this invention, a host cell is transformed with the vector. According to one embodiment of this invention, the host cell is a prokaryotic cell. According to an alternative embodiment of this invention, the host cell is a eukaryotic cell. According to another embodiment, the present invention provides an isolated cell line comprising the provided nucleic acid.

The present invention also provides a method for generating crystallization variants of an HIV-RT-NNRTI complex, comprising the steps of (a) truncating at least one terminus of HIV-RT; (b) reducing surface lysine acid regions; and (c) mutating at least one amino acid residue, thereby altering lattice contact from the non-mutated residue. According to one embodiment, step b comprises reducing surface glutamic acid regions. According to another embodiment, step b comprises mutating lysine to alanine. According to still another embodiment, step b comprises mutating glutamic acid to alanine. According to a further embodiment, step c is systematic mutagenesis. According to a preferred embodiment, step c is achieved by methylated overlap extension ligation independent cloning (MOE-LIC). According to still yet another embodiment, the provided method further comprises the step of selecting mutant HIV-RT for enzymatic activity. Still a further embodiment of the method comprises the step of crystallizing the mutant HIV-RT. It is understood that it is important to minimize mutation of conserved amino acid residues. According to a further embodiment, the method further comprises the step of determining the three dimensional crystal structure of the mutant HIV-RT-NNRTI complex. According to a preferred embodiment the resolution is determined to better than about 3.0 Å resolution. According to a most preferred embodiment, the resolution is determined to better than about 2.0 Å resolution. The present invention also provides an HIV-RT-NNRTI complex produced by the provided method. According to a preferred embodiment, the NNRTI is a DAPY compound. According to a most preferred embodiment, the DAPY compound is selected from the group consisting of TMC278 and TMC125. The present invention also provides a L100/K103N and K103N/y181C double mutant in the p66 subunit.

The present invention provides a method for identifying HIV-RT inhibitor solvent molecules comprising the steps of: (a) soaking a small molecule fragment into a crystallization variant generated by the provided method, thereby forming an HIV-RT complex with the molecule; (b) determining three dimensional structure of the complex; and (c) determining HIV-RT enzyme activity.

The present invention provides a plasmid containing both the p66 and p51 subunits of RT under separate promoters. The plasmid was designed to allow facile manipulation of the subunits independently using standard molecular cloning techniques. Mutagenesis of HIV-RT was used to generate constructs capable of producing crystals to diffract X-rays to high resolution. The techniques of mutagenesis, expression, purification, crystallization, and X-ray diffraction data collection were performed in iterative cycles. The iterative search led to the invention of a construct of RT that is biologically active and diffracts X-rays to high resolution (1.8 Å resolution). The construct used for crystallization has sequence beginning with GPISP sequence after proteolytic removal of MAHHHHHHALEVLFQ using the HRV14 C3 protease. The crystals of this construct, RT52A, in complexes with several NNRTIs have diffracted X-rays to better than 2.0 Å resolution. The crystals have symmetry of space group C2 with approximate cell parameters a=160-165, b=71-74, c=107-114 A, any=90 and 0=99-103′. This unit cell is novel when compared with all crystal structures of HIV-1 RT available in the Protein Data Bank. The present invention further provides Drug resistance mutations that were introduced into the plasmid and high resolution structures of mutant RT. Double mutants are provided that develop high resistance to most NNRTIs. Protein RT51A contains two mutations in p66: L100I and K103N. Protein RT55A contains two mutations in p66: K103N and Y181C. These mutant RTs have also been successfully crystallized and structurally studied.

It is understood that the structures provided by the present invention using the provided methods are of highly improved quality compared to those available in the PDB and, therefore, provide reliable information on inhibitor binding pocket and ligand protein interactions. It is understood by those of skill in the art that the provided HIV-RT construct, mutants, described crystal form, and determined 3-D structure information in molecular docking and other computational tools to generate new lead RT inhibitors targeting polymerization/RNase H activity and in optimization of lead compounds. Moreover, high resolution crystal structures obtained using the construct(s) provided by the present invention provide a method to locate solvent molecules unambiguously which otherwise was not feasible using available crystal forms and techniques prior to this invention. The present invention thereby provides a fragment-based drug discovery method. According to the provided method, many small molecule fragments or cocktails of fragments (obtained commercially or through scientific collaborations) are soaked into the above described crystals of engineered RT. Binding of certain chemical fragments identify novel drug binding sites or novel modes of inhibitor binding to existing drug-binding sites.

The provided plasmid produces engineered RT which is enzymatically active and yields crystals that diffract X-rays to significantly high resolution (better than 2 Å). The solution structures of RT and its complexes with different inhibitors are critical for design of RT inhibitors and availability of this construct and crystal form dramatically accelerates the rate of successful drug identification.

The provided plasmid produces a novel heterodimeric protein consisting of the two subunits p66 and p51. The amino-terminus of p66 begins as MVPISP while the amino terminus of p51 contains a cleavable purification tag which after cleaving leaves GPISP as the amino-terminus. The carboxy-terminus of p66 ends at residue 555 and the carboxy-terminus of p51 ends at residue 428. The following mutations are present in p66: K172A, K173A, and C280S. p51 also has the C280S mutation.

These features in combination allow for improved resolution of X-ray crystal structures of the protein with inhibitors. Typical resolution of HIV-1 reverse transcriptase with nonnucleoside reverse transcriptase inhibitors (NNRTI's) is 2.5-3.0 A resolution. With the engineered protein, called RT52A, resolution of 1.8-2.4 A is common. In addition to improved resolution the provided protein crystallizes in a fraction of the time it would take the non-engineered protein to crystallize. It now takes hours to days to crystallize instead of days to weeks.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Cells, ed. (1994)]; “Current Protocols in Immunology” Volumes [Coligan, J. E., ed. (1994)]; “Oligonucleotide Synthesis” (M. J. Gait ed. 1984); “Nucleic Acid Hybridization” [B. D. Hames S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

The amino acid residues described herein are preferred to be in the “L” isomeric form. However, residues in the “D” isomeric form can be substituted for any L-amino acid residue, as long as the desired functional property of immunoglobulin binding is retained by the polypeptide. NH₂ refers to the free amino group present at the amino terminus of a polypeptide. COOH refers to the free carboxy group present at the carboxy terminus of a polypeptide. In keeping with standard polypeptide nomenclature, abbreviations for amino acid residues are used as shown in shown in the following Table of Correspondence:

TABLE OF CORRESPONDENCE

| SYMBOL | | |
|---|---|---|
| 1-Letter | 3-Letter | AMINO ACID |
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above Table is presented to correlate the three-letter and one-letter notations, which may appear alternately herein.

It should also be noted that in addition to the standard IUPAC one-letter code for the nucleotides of DNA the following code is used herein including letters for ambiguity as follows: M is A or C; R is A or G; W is A or T; S is C or G; Y is C or T; K is G or T; V is A, C or G; H is A, C or T; D is A, G or T; B is C, G or T; and N is G, A, T or C.

A “replicon” is any genetic element (e.g., plasmid, chromosome, virus) that functions as an autonomous unit of DNA replication in vivo; i.e., capable of replication under its own control. A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Another feature of this invention is the expression of the DNA sequences disclosed herein. As is well known in the art, DNA sequences may be expressed by operatively linking them to an expression control sequence in an appropriate expression vector and employing that expression vector to transform an appropriate unicellular host.

Such operative linking of a DNA sequence of this invention to an expression control sequence, of course, includes, if not already part of the DNA sequence, the provision of an initiation codon, ATG, in the correct reading frame upstream of the DNA sequence.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMB9 and their derivatives, plasmids such as RP4; phage DNAS, e.g., the numerous derivatives of phage λ, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences—sequences that control the expression of a DNA sequence operatively linked to it—may be used in these vectors to express the DNA sequences of this invention. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage λ, the control regions of fd coat protein, the promoter for 3-phosphoglycerate kinase or other glycolytic enzymes, the promoters of acid phosphatase (e.g., Pho5), the promoters of the yeast α-mating factors, and other sequences known to control the expression of genes of prokaryotic or eukaryotic cells or their viruses, and various combinations thereof.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, R1.1, B-W and L-M cells, African Green Monkey kidney cells (e.g., COS 1, COS 7, BSC1, BSC40, and BMT10), insect cells (e.g., Sf9), and human cells and plant cells in tissue culture.

It will be understood that not all vectors, expression control sequences and hosts will function equally well to express the DNA sequences of this invention. Neither will all hosts function equally well with the same expression system. However, one skilled in the art will be able to select the proper vectors, expression control sequences, and hosts without undue experimentation to accomplish the desired expression without departing from the scope of this invention. For example, in selecting a vector, the host must be considered because the vector must function in it. The vector's copy number, the ability to control that copy number, and the expression of any other proteins encoded by the vector, such as antibiotic markers, will also be considered.

In selecting an expression control sequence, a variety of factors will normally be considered. These include, for example, the relative strength of the system, its controllability, and its compatibility with the particular DNA sequence or gene to be expressed, particularly with regard to potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the DNA sequences to be expressed, and the ease of purification of the expression products.

Considering these and other factors a person skilled in the art will be able to construct a variety of vector/expression control sequence/host combinations that will express the DNA sequences of this invention on fermentation or in large-scale animal culture.

Transcriptional and translational control sequences are DNA regulatory sequences, such as promoters, enhancers, polyadenylation signals, terminators, and the like, that provide for the expression of a coding sequence in a host cell. A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide,” as used generally herein, such as in referring to probes prepared and used in the present invention, is defined as a molecule comprised of two or more ribonucleotides, preferably more than three. Its exact size will depend upon many factors, which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

“Degenerate to” is meant that a different three-letter codon is used to specify a particular amino acid. It is well known in the art that the following codons can be used interchangeably to code for each specific amino acid:

| | |
|---|---|
| Phenylalanine (Phe or F) | UUU or UUC |
| Leucine (Leu or L) | UUA or UUG or CUU or CUC or CUA or |
| | CUG |
| Isoleucine (Ile or I) | AUU or AUC or AUA |
| Methionine (Met or M) | AUG |
| Valine (Val or V) | GUU or GUC of GUA or GUG |
| Serine (Ser or S) | UCU or UCC or UCA or UCG or AGU |
| | or AGC |
| Proline (Pro or P) | CCU or CCC or CCA or CCG |
| Threonine (Thr or T) | ACU or ACC or ACA or ACG |
| Alanine (Ala or A) | GCU or GCG or GCA or GCG |
| Tyrosine (Tyr or Y) | UAU or UAC |
| Histidine (His or H) | CAU or CAC |
| Glutamine (Gln or Q) | CAA or CAG |
| Asparagine (Asn or N) | AAU or AAC |
| Lysine (Lys or K) | AAA or AAG |
| Aspartic Acid (Asp or D) | GAU or GAC |
| Glutamic Acid (Glu or E) | GAA or GAG |
| Cysteine (Cys or C) | UGU or UGC |
| Arginine (Arg or R) | CGU or CGC or CGA or CGG or AGA or |
| | AGG |
| Glycine (Gly or G) | GGU or GGC or GGA or GGG |
| Tryptophan (Trp or W) | UGG |
| Termination codon | UAA (ochre) or UAG (amber) or UGA (opal) |

It should be understood that the codons specified above are for RNA sequences. The corresponding codons for DNA have a T substituted for U.

Mutations can be made in the nucleotide sequence encoding SEQ. ID. NO:1 or SEQ. ID. NO:2 or other sequences described herein, such that a particular codon is changed to a codon which codes for a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative changes which do not significantly alter the activity or binding characteristics of the resulting protein. The following is one example of various groupings of amino acids:

Amino Acids with Nonpolar R Groups

### Alanine

### Valine

### Leucine

### Isoleucine

### Proline

### Phenylalanine

### Tryptophan

### Methionine

Amino Acids with Uncharged Polar R Groups

### Glycine

### Serine

### Threonine

### Cysteine

### Tyrosine

### Asparagine

### Glutamine

Amino Acids with Charged Polar R Groups (Negatively Charged at pH 6.0)
Aspartic acid
Glutamic acid

### Basic Amino Acids (Positively Charged at pH 6.0)

### Lysine

### Arginine

### Histidine (at pH 6.0)

Another Grouping May be Those Amino Acids with Phenyl Groups:

### Phenylalanine

### Tryptophan

### Tyrosine

### Another Grouping May be According to Molecular Weight (i.e., Size of R Groups):

| | |
|---|---|
| Glycine | 75 |
| Alanine | 89 |
| Serine | 105 |
| Proline | 115 |
| Valine | 117 |
| Threonine | 119 |
| Cysteine | 121 |
| Leucine | 131 |
| Isoleucine | 131 |
| Asparagine | 132 |
| Aspartic acid | 133 |
| Glutamine | 146 |
| Lysine | 146 |
| Glutamic acid | 147 |
| Methionine | 149 |
| Histidine (at pH 6.0) | 155 |
| Phenylalanine | 165 |
| Arginine | 174 |
| Tyrosine | 181 |
| Tryptophan | 204 |

### Particularly Preferred Substitutions are:

Lys for Arg and vice versa such that a positive charge may be maintained;

Glu for Asp and vice versa such that a negative charge may be maintained;

Ser for Thr such that a free —OH can be maintained; and

Gln for Asn such that a free NH₂ can be maintained.

Two amino acid sequences are “substantially homologous” when at least about 70% of the amino acid residues (preferably at least about 80%, and most preferably at least about 90 or 95%) are identical, or represent conservative substitutions.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20° C. below the predicted or determined Tₘ with washes of higher stringency, if desired.

Media useful for the preparation of these compositions are both well-known in the art and commercially available and include synthetic culture media, inbred mice and the like. An exemplary synthetic medium is Dulbecco's minimal essential medium (DMEM; Dulbecco et al., Virol. 8:396 (1959)) supplemented with 4.5 gm/l glucose and 20 mm glutamine.

It is contemplated that the proteins, peptides, nucleic acids, vectors and virus particles of this invention can be administered to a subject to impart a therapeutic or beneficial effect. Therefore, the proteins, peptides, nucleic acids, vectors and particles of this invention can be present in a pharmaceutically acceptable carrier.

“Pharmaceutically acceptable” means that a material that is not biologically or otherwise undesirable, i.e., the material may be administered to a subject, along with the nucleic acid or vector of this invention, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art (see, e.g., Remington's Pharmaceutical Science; latest edition).

Pharmaceutical formulations of this invention, such as vaccines, of the present invention can comprise an immunogenic amount of the virus particles as disclosed herein in combination with a pharmaceutically acceptable carrier. An “immunogenic amount” is an amount of the virus particles sufficient to evoke an immune response (humoral and/or cellular immune response) in the subject to which the pharmaceutical formulation is administered.

Exemplary pharmaceutically acceptable carriers include, but are not limited to, sterile pyrogen-free water and sterile pyrogen-free physiological saline solution.

Pharmaceutical formulations for the present invention can include those suitable for parenteral (e.g., subcutaneous, intradermal, intramuscular, intravenous and intraarticular) administration. Alternatively, pharmaceutical formulations of the present invention may be suitable for administration to the mucous membranes of a subject (e.g., intranasal administration). The formulations may be conveniently prepared in unit dosage form and may be prepared by any of the methods well known in the art.

Thus, the present invention provides a method for delivering nucleic acids and vectors (e.g., virus particles) encoding the proteins of this invention to a cell, comprising administering the nucleic acids or vectors to a cell under conditions whereby the nucleic acids are expressed, thereby delivering the proteins of this invention to the cell. The nucleic acids can be delivered as naked DNA or in a vector (which can be a viral vector) or other delivery vehicles and can be delivered to cells in vivo and/or ex vivo by a variety of mechanisms well known in the art (e.g., uptake of naked DNA, viral infection, liposome fusion, endocytosis and the like). The cell can be any cell which can take up and express exogenous nucleic acids.

As used herein, “pM” means picomolar, “nM” means nanmolar, “uM” means micromolar, “mM” means millimolar, “ul” means microliter, “ml” means milliliter, “l” means liter.

As used herein, the term “synthetic amino acid” means an amino acid which is chemically synthesized and is not one of the 20 amino acids naturally occurring in nature. As used herein, the terms “non-natural amino acid” and “unnatural amino acid” means an amino acid, which is not one of the 20 amino acids naturally occurring in nature. Thus, a synthetic amino acid is an unnatural amino acid.

As used herein, the term “biosynthetic amino acid” means an amino acid found in nature other than the 20 amino acids commonly described and understood in the art as “natural amino acids.” Examples of “non-amide isosteres” include but are not limited to secondary amine, ketone, carbon-carbon, thioether, and ether moieties.

As used herein, the term “non-natural peptide analog” means a variant peptide comprising a synthetic amino acid. As used herein, “NMR” means nuclear magnetic resonance, “ESMS” means electrospray mass spectrometry; “CBD” means chitin binding, domain; “SH2” means src homology type-2 domain; “Abl” means human Abelson protein tyrosine kinase, “GST” means glutathione S-transferase; “HSQC” means heteronuclear single-quantum correlation spectroscopy. “HPLX” means high pressure liquid chromatography; “PhSH” means thiophenol, “BzlSH” means benzyl mercaptan; standard single and triple letter codes for amino acids, and single letter codes for nucleic acids are used throughout.

A “segment” as the term is used herein, consists of a portion of a protein or peptide primary amino acid sequence. Such a segment as used herein may be generated by proteolytic cleavage, chemical cleavage or physical disruption. Alternatively, such a segment may be generated by an expression vector or by an in vitro translation of an RNA transcript or portion thereof. Such a segment may assume a structural conformation or folding pattern which is unique to the segment or which represents the conformation of the segment in the complete protein or peptide.

A “domain” as used herein, is a portion of a protein that has a tertiary structure. The domain may be connected to other domains in the complete protein by short flexible regions of polypeptide. Alternatively, the domain may represent a functional portion of the protein.

As used herein, amino acid residues are preferred to be in the “L” isomeric form. However, residues in the “D” isomeric form can be substituted for any L-amino acid residue, as long as the desired functional property of immunoglobulin-binding is retained by the polypeptide. NH₂ refers to the free amino group present at the amino terminus of a polypeptide. COOH refers to the free carboxy group present at the carboxy terminus of a polypeptide. Abbreviations for amino acid residues are used in keeping with standard polypeptide nomenclature delineated in J. Biol. Chem., 243:3552-59 (1969).

It should be noted that all amino-acid residue sequences are represented herein by formulae whose left and right orientation is in the conventional direction of amino-terminus to carboxy-term inns. Furthermore, it should be noted that a dash at the beginning or end of an amino acid residue sequence indicates a peptide bond to a further sequence of one or more amino-acid residues.

Amino acids with nonpolar R groups include: Alanine, Valine, Leucine, Isoleucine, Proline, Phenylalanine, Tryptophan and Methionine. Amino acids with uncharged polar R groups include: Glycine, Serine, Threonine, Cysteine, Tyrosine, Asparagine and Glutamine. Amino acids with charged polar R groups (negatively charged at pH 6.0) include: Aspartic acid and Glutamic acid. Basic amino acids (positively charged at pH 6.0) include: Lysine, Arginine and Histidine (at pH 6.0). Amino acids with phenyl groups include: Phenylalanine, Tryptophan and Tyrosine. Particularly preferred substitutions are: Lys for Arg and vice versa such that a positive charge may be maintained; Glu for Asp and vice versa such that a negative charge may be maintained; Ser for Thr such that a free —OH can be maintained; and Gln for Asn such that a free NH₂ can be maintained. Amino acids can be in the “D” or “L” configuration. Use of peptidomimetics may involve the incorporation of a non-amino acid residue with non-amide linkages at a given position.

Amino acid substitutions may also be introduced to substitute an amino acid with a particularly preferable property. For example, a Cys may be introduced as a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces β-turns in the protein's structure.

The detectable marker labels most commonly employed for these studies are radioactive elements, enzymes, chemicals which fluoresce when exposed to ultraviolet light, and others.

A number of fluorescent materials are known and can be utilized as labels. These include, for example, fluorescein, rhodamine, auramine, Texas Red, AMCA blue and Lucifer Yellow. A particular detecting material is anti-rabbit antibody prepared in goats and conjugated with fluorescein through an isothiocyanate.

The proteins and peptides of the present invention can also be labeled with a radioactive element or with an enzyme. The radioactive label can be detected by any of the currently available counting procedures. The preferred isotope may be selected from ³H, ¹³C, ¹⁵N, ¹⁴C, ³²P, ³⁵S, ³⁶Cl, ⁵¹Cr, ⁵⁷Co, ⁵⁷Co, ⁵⁹Fe, ⁹⁰Y, ¹²⁵I, ¹³¹I, and ¹⁸⁶Re.

Enzyme labels are likewise useful, and can be detected by any of the presently utilized calorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods.

A basic description of nucleic acid amplification or PCR (polymerase chain reaction) is described in Mullis, U.S. Pat. No. 4,683,202, which is incorporated herein by reference. The amplification reaction uses a template nucleic acid contained in a sample, two primer sequences and inducing agents. The extension product of one primer when hybridized to the second primer becomes a template for the production of a complementary extension product and vice versa, and the process is repeated as often as is necessary to produce a detectable amount of the sequence.

The inducing agent may be any compound or system which will function to accomplish the synthesis of primer extension products, including enzymes. Suitable enzymes for this purpose include, for example, E. coli DNA polymerase thermostable Taq DNA polymerase, Klenow fragment of E. coli DNA polymerase I, T4 DNA polymerase, other available DNA polymerases, reverse transcriptase and other enzymes which will facilitate combination of the nucleotides in the proper manner to form amplification products. The oligonucleotide primers can be synthesized by automated instruments sold by a variety of manufacturers or can be commercially prepared based upon the nucleic acid sequence of this invention.

As used herein, the term “chip” means any solid support including, but not limited to silicon, glass, polypropylene, polystyrene, cellulose, plastic and paper. Accordingly, the term “protein chip” means a protein covalently bound to a solid support including, but not limited to silicon, glass, polypropylene, polystyrene, cellulose, plastic and paper. The “protein” component of a protein chip as used herein is the ligation product of an oligopeptide and a recombinantly expressed protein or portion thereof, the peptide being the component covalently bound to the solid support. Additionally, as used herein, the term “antibody chip” means an antibody or the antigen-binding portion thereof covalently bound to a solid support as the ligation product of an oligopeptide and a recombinantly expressed antibody protein or portion thereof, the peptide being the component covalently bound to the solid support. Furthermore, as used herein, the term “antigen chip” means an antigen covalently bound to a solid support as the ligation product of an oligopeptide and a recombinantly expressed antigenic protein or portion thereof, the peptide being the component covalently bound to the solid support. Moreover, the term “protein chip protein” refers to the protein component of the protein chip which is the ligation product produced by the methods disclosed by the present invention.

### EXAMPLES

### Material and Methods

### Expression Vector and Mutation Construction

The RT coding DNA from the Q258C-RT construct (Sarafianos et al., 2003) was ligation independent cloned into pCDF-2 Ek/LIC with the LIC Duet™ Minimal Adaptor (Novagen) according to manufacturer's recommendations. The termini of the p66 insert (ORF-2) contained the restrictions sites for the enzymes NdeI and XhoI while the termini of the p51 insert (ORF-1) contained NcoI and SacI restriction sites (New England Biolabs). To remove any residues added to the expressed protein by the vector, NdeI and NcoI were used to remove all DNA between the start codon and the insert. The RT encoding dual expression vector is call pRT1.

Mutagenesis was completed using methylated overlap extension ligation independent cloning (MOE-LIC). The following methylated primers were used:

(SEQ ID NO: 122)
revcasLIC3-GCCCGAAGAGGAGC[2′OMeG]CCGGTTTCTTTACCAGA
CTCGAG;
(SEQ ID NO: 123)
forvectLIC3-CTCCTCTTCGGGC[2′OMeC]CGCCAGCACATGGACTC
G;
(SEQ ID NO: 124)
RevvectLIC2-GGAGAAAGCCC[2′0MeG]GGTATGGCATGATAGCGC
C;
(SEQ ID NO: 125)
orf2vrevLIC2-ACGCGGGCGGCCG[2′OMeU]GGATCCTTACGCCCCG
C;
(SEQ ID NO: 126)
ForcesLIC-CGGGCTTTCTCCT(2′OMeC)CTCTCCCTTATGCGACTC
C;
(SEQ ID NO: 127)
orfcasforLIC-CGGCCGCCCGCGTG(2′OMeG)TTGATCTCGATCCCG
CG;
(SEQ ID NO: 128)
orf2vrevLIC-CACGCGGGCGGCCG(2′OMeT)GGATCCCCCCGGGTC
C.

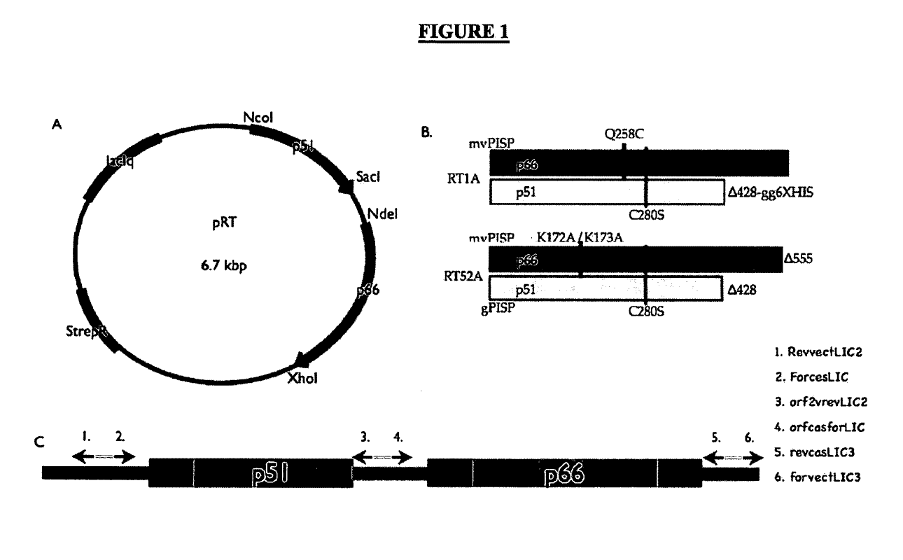

See FIG. 1 for the location and pairing of these primers on pRT1.

To minimize false positive colonies the vector was restriction digested with the appropriate restriction enzymes to remove the ORF protein coding DNA that was to be replaced (Neal and SacI for ORF-1 or NdeI and XhoI for ORF-2). For ORF-2 5 μl of vector (250 ng/μl) were digested in a 20 μl volume with 1 μl NdeI (20,000 units/ml) and 1 μl XhoI (20,000 units/ml) for one hour at 37° C. with NEBuffer2 (New England Biolabs). For p66, mutagenesis overlap extension PCR was performed using mutated overlap segments with the 2′-O-methylated primers orf2vrevLIC and revcasLIC3 to amplify the full insert with PfuUltra™ II Fusion HS DNA Polymerase (Stratagene). A typical overlap extension PCR was performed with 1 μl of each template, 1 μl (20 pmols) of each primer, 39 μl water, 1 μl (25 mM each) dNTPs, 5 μl 10× PfuUltra buffer, and 1 μl PfuUltra™ II Fusion HS DNA Polymerase (Stratagene). The PCR program is listed: 3 minutes at 95° C.; followed by 5 cycles of 1 minute at 95° C., 1 minute at 50° C., and 30 seconds at 72° C.; 30 cycles of 30 seconds at 95° C., 30 seconds at 53° C., and 45 seconds at 72° C.; ending with a final extension step of 10 minutes at 72° C.

In a separate reaction tube, the digested vector was PCR amplified with oligonucleotides orf2vrevLIC2 and forvectLIC3. The vector PCR was performed with 0.5 μl of the template (50 ng digested vector), 1 μl (20 pmols) of each primer, 40.5 μl water, 1 μl (25 mM each) dNTPs, 5 μl 10× PfuUltra buffer, and 1 μl PfuUltra™ II Fusion HS DNA Polymerase (Stratagene). The PCR program is listed: 3 minutes at 95° C.; followed by 30 cycles of 30 seconds at 95° C., 30 seconds at 54° C., and 90 seconds at 72° C.; ending with a final extension step of 10 minutes at 72° C.

The PCR products were then gel purified from a 0.5 or 1% agarose gel using the QIAquick gel extraction kit (see Appendix). The concentrations were determined by UV absorbance and 0.04 pmols of vector and insert were mixed at a 1:1 insert to vector molar ratio in a buffer containing 25 mM Tris pH 8.0, 5 mM MgCl₂, 0.025 mg/ml BSA, and 2.5 mM DTT in a 20 μl volume. The mixture was heated to 70° C. and cooled slowly over two hours in a water bath. Once cooled to ˜40° C., 1 μl of 25 mM EDTA was added and the mixture incubated at room temperature for 5 minutes before being desalted by Centri-Sep column (Princeton Separations) or ethanol precipitation (Donahue et al., 2002). 5 μl of desalted annealed DNA was added to electrocompetent NovaBlue cells (Novagen) and electroporated according to manufacturer's recommendations. The resulting colonies were tested by colony PCR and miniprepped using QIAprep Spin Miniprep kit (Qiagen) according to manufacturer's recommendations.

### Expression and Purification of HRV4 3C Protease

HRV14 3C protease was expressed in BL2′-CodonPlus®-RIL competent cells and grown on Luria-broth (LB) agar plates containing 35 mg/liter streptomycin and 0.1% glucose. A single colony was grown overnight in LB+35 mg/liter streptomycin and 0.5% glucose at 37° C. with shaking. The overnight culture was then inoculated in a 100-fold dilution, and the solution was incubated at 37° C. with shaking. Typical LB volume was 0.5-1.0 liters. When an OD₆₀₀ of 0.9 was reached, the cells were cooled to room temperature and induced with 0.5 mM isopropyl β-D-1-thiogalactopyranoside and incubated for 17 hours at 17° C. prior to pelleting and storage at −80° C. Nickel column purification was performed exactly as described for RT. Concentration is measured using the Bio-Rad Protein Assay (Bio-Rad) and the protein is stored at 1 mg/ml in a 50% glycerol solution.

### Expression and Purification of RT

Plasmids were transformed into BL21-CodonPlus®-RIL competent cells and grown on LB-agar plates containing 35 mg/liter streptomycin and 0.1% glucose. A single colony was grown overnight in LB+35 mg/liter streptomycin and 0.5% glucose at 37° C. with shaking. The overnight culture was then inoculated in a 100 fold dilution, and the solution was incubated at 37° C. with shaking. Typical LB volume was 250 ml to 4 liters. When an OD₆₀₀ of 0.9 was reached, the cells were induced with 1 mM IPTG and incubated for three hours prior to pelleting and storage at −80° C.

Nickel column purification was performed according to the manufacturer's recommendations (Qiagen) with the following modifications: no lysozyme was added to the lysate, 600 mM NaCl instead of 300 mM was used in each of the standard buffers, 0.1% Triton X-100 was added to the lysate and wash buffers, and an extra high-salt wash step was performed with 1.2 M NaCl. Following elution the yield of RT was checked by OD₂₈₀ (OD₂₈₀/3.1×dilution factor) and a 1:100 by weight ratio of HRV14 3C protease to RT is added. The protease treated solution was incubated at 4° C. overnight. The solution was buffer exchanged 20-fold into buffer A (50 mM diethanolamine pH 8.9) using an Amicon Ultra-15 Centrifugal unit with an Ultracell-30 membrane (Millipore). The solution was filtered with a 0.22 micron filter, and 10-20 mg was loaded onto a monoQ column (Amersham Biosciences) equilibrated with buffer A. The column was washed with buffer A and the samples eluted during a one hour gradient from 0 to 25% buffer B (buffer A+1 M NaCl) with a flow rate of 4 ml/minute. The monoQ column purification step effectively removed the protease. The RT was buffer exchanged and concentrated to 20 mg/ml in 10 mM Tris pH 8.0 and 75 mM NaCl. The concentrated RT was aliquoted and stored at −80° C. or 4° C. for immediate crystallization.

### Crystallization

The RT was screened unliganded, with a 2.5-fold molar excess of NNRTI, or with a 5-fold molar excess of RNHI using the hanging drop vapor diffusion method (0.17 mM RT with 0.425 mM NNRTI or 0.85 mM RNHI). Depending on the number of samples being screened, EasyXtal DG-Tools (Qiagen) or Linbro Plates (Hampton Research) crystallization trays were used for screening. Drop size for initial screening was 1 μl protein plus 1 μl reservoir solution. The well contained 500 μl of solution for Linbro Plates and 750 μl for EasyXtal DG-Tools. For the initial screening a RT-reference screen was used. Based on visually identified crystal hits, further is optimization was used. RT52A crystals were produced in a matrix of 24 conditions from 9-12% PEG 8000, 50 mM imidazole pH 6.0-6.8, 10 mM spermine, 15 mM MgSO₄, and 100 mM ammonium sulphate. The origin and age of PEG 8000 used was found to be very important. PEGs will react to light and oxygen, which results in small PEG products and a drop in pH. The change in pH can have a dramatic effect on the final pH of the crystallization solution (e.g. pH of 6.5 to 6.0). With new lots of PEG 8000, 4% PEG 400 was used as an additive with an appropriate decrease in the pH of buffer was used to reproduce crystals with old lots of PEG 8000. In addition to the matrix, the Additive Screen (Hampton Research) was used with the base solution 11% PEG 8000, 50 mM imidazole pH 6.4, 10 mM spermine, 15 mM MgSO₄, and 100 mM ammonium sulfate. All successful crystallization experiments were performed at 4° C. with protection from light and temperature fluctuations by placement inside a Styrofoam box.

Drops that did not contain crystals after three days were microseeded with crushed RT52A/NNRTI crystals. Microseeding was performed by crushing several preferably otherwise unusable RT52A/NNRTI crystals on a glass plate followed by use of the Seed Bead kit (Hampton Research) according to manufacturer's recommendations. Total volume of well solution used was typically 100 μl. Several dilutions of this seed stock were made and tested on a subset of the crystallization drops that were to be seeded. A 30-gauge needle was used to streak seed the drops. The seed solutions were stored overnight in a drawer at 4° C. and the seeded drops were checked after 24 hours. Based on the number of crystals in the seeded drops, further seeding was performed. After all the drops were seeded the seed stock was stored for future use at −80° C.

NNRTI/RT52A crystals were found to be very stable with no loss in X-ray diffraction quality after four months at 4° C. Unliganded and RNHI/RT52A crystals however were found to deteriorate both in X-ray diffraction and visual qualities with in one week of appearing. RT69A/RNHI crystals were stable for weeks.

### Data Collection

Crystals of RT52A were flash-cooled by immersion into liquid nitrogen after briefly dunking the crystal into cryoprotection solution containing well solution plus 27% ethylene glycol and the inhibitor at the same concentration as in the hanging drop. Best results were found when using MicroMounts (MiTeGen) for mounting the crystals. Data for screening and data set collection were obtained at Advanced Photon Source (APS) at Argonne National Laboratory (ANL), SER-CAT beamline 191D, Cornell High Energy Synchrotron Source (CHESS) F1 and A1 beamlines, and National Synchrotron Light Source (NSLS) beamlines X25 and X29. The diffraction data were indexed, processed, scaled and merged using HKL2000I (Otwinowski et al., 1999). The resolution of the data was estimated using the last resolution shell with preferred values for completeness, R-merge, and the ratio of I to σ(I).

### Dynamic Light Scattering

Samples were tested using the DynaPro-MS800 dynamic light scattering/molecular sizing instrument (Protein Solutions, Inc.). 20 μl of 1 mg/ml RT in 10 mM Tris pH 8.0 and 75 mM NaCl was tested after centrifugation at 14,000 g for 2 minutes. Each experiment consisted of no less then 25 measurements. Data analyses were performed using DynaPro Instrument Control Software for Molecular Research DYNAMICS (version 5.26.60).

### CD Spectroscopy

RT samples were diluted in 2 mM HEPES (pH 8.2) and 75 mM NaCl to a final concentration of 0.12 mg/ml (0.5 ml total volume) and centrifuged at 15,000 g for 2 minutes before measurements. CD spectra were recorded before and after melt from 200 to 260 nm on an AVIV Circular Dichroism Spectrometer Model 215. The thermal stability assay was performed starting at 4° C. and increased in 0.2° C. increments to 70° C. with 5 second measurements at 222 nm taken at each increment.

### RT Activity Assays

### DNA-Dependent DNA Polymerase (DDDP) Processivity Assay

The DDDP processivity assay was done by Paul Boyer (laboratory of Stephen Hughes, NCI-Frederick Cancer Research and Development Center, Frederick, Md.) as previously described (Boyer et al., 2002). The WT HIV-1 RT was produced as described previously (Boyer et al., 1999). The primer-47 (New England Biolabs) was 5′-end labeled and then annealed to single-strand M13 mp 18 DNA (New England Biolabs). The final concentration of template-primer (T/P) in each reaction mixture was approximately 2.5 nM; the RT was in molar excess (85 nM). The cold trap, poly(rC).oligo(dG), was added in excess relative to RT (300 nM) after the RT was allowed to bind to the labeled T/P. The extension products were suspended in 2× gel loading buffer (Ambion) and heated at 65° C. to denature the samples. A 15 hour electrophoresis of loaded samples was performed on an alkaline agarose gel (Sambrook et al., 1989). The products were visualized by exposure to X-ray film.

### RNase H Assay

Activity measurements were done by Paul Bayer (NCI-Frederick Cancer Research and Development Center, Frederick, Md.) as in Boyer et al., (2004). Briefly, The RNA oligonucleotides were 5′-end labeled and then annealed to synthetic DNA oligonucleotides by heating and slow cooling. A 0.2 μM concentration of T/P was suspended in a total reaction volume of 12 μl containing 25 mM Tris (pH 8.0), 50 mM NaCl, 5.0 mM MgCl2, 100 μg of bovine serum albumin/ml, 10 mM CHAPS, and 1 U of Superasin (Ambion)/μl. The reactions were initiated by the addition of the 75 ng of the indicated RT and were incubated at 37° C. Aliquots were removed at the indicated time points, and the reaction was halted by addition of 2× gel loading buffer. The reaction products were fractionated on a 15% polyacrylamide sequencing gel. Products were visualized by exposure to X-ray film.

### Expression/Purification/Crystallization

RT52A was expressed and purified as described. The NNIBP mutants were produced by site-directed mutagenesis of RT52A as described. The NNIBP mutants were expressed and purified in the same manner as RT52A. Crystallization was performed using the hanging drop vapor diffusion method. RT52A/TMC278 was crystallized by adding 1 μl of RT52A/TMC278 complex at 20 mg/ml to an equal volume of well solution (11% PEG 8000, 15 mM MgSO₄, 10 mM spermine, 100 mM ammonium sulfate, 50 mM imidazole, pH 6.8, and 60 mM sodium formate). L100′-K103N/TMC278 was crystallized by adding 1 μl of L100I-K103N/TMC278 complex at 20 mg/ml to an equal volume of well solution (8% PEG 8000, 15 mM MgSO₄, 10 mM spermine, 100 mM ammonium sulfate, and 50 mM imidazole, pH 6.2). K103N-Y181C/TMC278 was crystallized by adding 1 μl of K103N-Y181C/TMC278 complex at 20 mg/ml to an equal volume of well solution (12% PEG 8000, 15 mM MgSO₄, 10 mM spermine, 100 mM ammonium sulfate, and 50 mM sodium citrate, pH 5.0).

### Data Collection and Structure Solution

Crystals of RT52A were flash-cooled by immersion into liquid nitrogen after briefly dunking the crystal into cryoprotection solution containing well solution plus 27% ethylene glycol and the inhibitor at the same concentration as in the hanging drop. X-ray diffraction data were collected at the Cornell High Energy Synchrotron Source (CHESS) F1, and Advanced Photon Source (APS) at Argonne National Laboratory, SER-CAT beamline 191D. The diffraction data were indexed, processed, and scaled using HKL2000 (Otwinowski et al., 1999), Structure determination was performed by Kalyan Das. The previously reported HIV-1 RT/R129385 (PDB code 1S9E) structure was used as a template in obtaining molecular replacement solutions for the RT52A/TMC278 complex structure. Rigid body refinement of the initial model, broken into 13 separate segments (one fragment per subdomain except for two fragments each from p66 and p51 fingers and palm) reduced the starting R-factors by about 4-5%, indicating significant interdomain rearrangments in the RT52A/TMC278 structure compared to that in the starting model. The final model for the complex was obtained after cycles of model building in 0 (Jones et al., 1991), COOT (Emsley and Cowtan, 2004), and restrained refinements using CNS 1.1 (Brunger et al., 1998) and REFMAC (Murshudov et al., 1997). Similar molecular replacement and refinement steps were used in obtaining the remaining two structures in which the RT52A/TMC278 structure was used as the template. The X-ray data, refinement statistics, and unit cell statistics are listed in Table 7.

### Gel Purification Protocol

Gel purification was found to give the highest yields when done with QIAquick Gel Extraction Kit (Qiagen). A modified protocol based on the manufacturer's recommendations: (1) Excise the DNA from the agarose gel with a clean, sharp razor. Minimize the size of the slice; (2) Weigh the gel slice in a colorless tube. Add 3 volumes (where volume in ml=mass in mg, example: 100 mg of gel is 100 μl of buffer) of Buffer QG if the gel slice mass is more than 200 mg otherwise add 0.6 ml; (3) Incubate at 50° C. for 10 minutes and vortex every 2-3 minutes. The gel slice must be completely dissolved; (4) Add 10 μl of 3 M sodium acetate, pH 5.0, and vortex. The solution would be yellow unless dye from the gel was in the slice; (5) Add 200 μl (or one gel volume) of isopropanol to the sample and vortex; (6) Place up to 750 μl of the solution into a QIAquick column. Centrifuge using a table-top centrifuge for 30 seconds. The bottom reservoir can be discarded and any additional solution can be added and centrifuged; (7) To wash add 0.75 ml of Buffer PE to the column and let sit for 3 minutes. Then centrifuge for 30 seconds; (8) Discard the flow through and centrifuge for 1 minute; (9) Place the QIAquick column into a clean 1.5 ml eppendorf. Elute by adding 50 μl of Buffer EB (10 mM Tris-Cl, pH 8.5, 50° C.) and letting the column sit at room temperature for one minute. A second elution will improve the yield by ˜30%. The second elution is done with 30 μl Buffer EB; (10) Reduce the volume of the solution by speedvacuuming the elution to the desired volume.

### Example 1

### Engineering HIV-1 Reverse Transcriptase for Improved Crystallization

1. Co-Expression and Mutant Cloning

A co-expression system was utilized to facilitate high-throughput subunit specific mutagenesis of RT. FIG. 1 shows the modified pCDF vector with p51 in open reading frame (ORF) 1 and p66 in ORF-2. Both ORFs have unique restriction sites for subunit specific cloning. This expression system allowed for high yield expression (−40 mg/liter) under standard expression conditions (FIG. 1A). In the expectation of creating many RT mutants, a rapid and inexpensive mutagenesis system was sought. Donahue et al. (2002) proposed a ligation independent cloning technique, which uses terminator primers to create 12-15 nucleotide overhangs on the insert and vector. The insert and vector are annealed and transformed into bacteria, thereby avoiding any post-PCR enzymatic steps. The terminating residue in the primer is a 2′-O-methylated nucleotide, which causes early termination of thermostable polymerases Taq or Pfu. There are two major problems with this technique: 1. The 2′-O-methylated primers cost ˜$100 per pair; and 2. The site of 2′-O-methylation has a 20% mutation rate.

A modified version of the terminator primer technique was developed for rapid mutagenesis of RT called methylated overlap-extension ligation independent cloning (MOE-LIC). MOE-LIC uses overlap-extension mutagenesis (Ho et al., 1989) and terminator primers outside the ORF to avoid unwanted mutagenesis of the coding or regulatory regions. Overlap-extension PCR can also be used to insert a completely new insert or modify the termini of a previously constructed insert (Horton et al., 1989). For the co-expression system a total of four terminator primer pairs were required at a cost of approximately $400, which could be used for over a thousand reactions (FIG. 1C).

2. Mutagenesis and Crystallization

A mutagenesis strategy to alter the crystallization of RT was developed to combine several methodologies: 1. Disrupt or enhance common crystal contacts in the known RT crystal forms; 2. Remove high B-factor patches, primarily disordered termini; 3. Reduce surface entropy by mutagenesis of lysine and glutamic acid patches to alanine; 4. Make use of the wealth of information of RT crystallization by the multiple research groups that have studied RT; and 5. Avoid mutating conserved residues. The starting template was chosen due to its successful application to DNA cross-linking studies (RT1A depicted in FIG. 1B). Table 3 shows the list of RT variants that were made for crystallization trials and the diffraction resolution of the crystals. Table 4 describes the 18 crystallization conditions used as a starting screen for each mutant. The 18 conditions were chosen for their successful use in previous RT crystallization trials (Clark et al., 1995, Chan et al., 2001. Rodgers et al., 1995, Hogberg et al., 1999, and unpublished data).

TABLE 3
List of all RT constructs and crystallization results.

| Construct | Template | Mutation | Clone/Express | Crystals/Diffraction |
|---|---|---|---|---|
| RT1A | | Paul Boyer's Q258C construct in pRT | | 10 Å |
| RT2A | RT1A | p66: K172R | | 10 Å |
| RT3A | RT1A | p66: K512Q | | no crystals |
| RT4a | RT1A | p66: E28A | | 10 Å |
| RT5A | RT1A | p51: L92 and G93 removed | | 10 Å |
| RT6A | RT1A | p66: delta 3 N-terminus | | 10 Å |
| RT7A | RT1A | p66: delta 4 N-terminus | | no crystals |
| RT8A | RT1A | p51: delta 3 N-terminus | | 10 Å |
| RT9A | RT1A | p51: delta 4 N-terminus | | 12 Å |
| RT10A | RT1A | p51: delta 427 | | no crystals |
| Ndtermini | RT1A | p66: delta 555; p51: delta 447 | | 9 Å |
| RT12A | RT1A | p66: N-terminus HRV3c-hexahistag delta | | |
| | | 555; p51: delta 428 | | |
| RT13A | RT1A | p51: N-terminus HRV3c-hexahistag delta 428; | Best expression | crystals - no diffraction |
| | | p66 delta 555 | of RT12A-14A | |
| RT14A | RT1A | p66: C-terminus hexahistag, p51: delta 428 | | |
| RT21A | RT13A | p66: K223A and E224A | cloning failed | |
| RT22A | RT13A | p66: F160S | | 4.0 Å |
| RT23A | RT13A | p66: K219A and K220A | | 3.7 Å |
| RT24A | RT13A | p66: K172A and K173A | | RT24A/TMC278 a = 99.95 b = 58.05 c = 515.459 |
| | | | | p522 3.5 Å |
| | | | | RT24A/CL32543 a = 73.51 b = 90.44 c = 126.17 |
| | | | | α = 105.39 β = 94.73 γ = 110.37 p1 3.3 Å |
| RT25A | RT13A | p66: K201A/E203A/E204A | cloning failed | |
| RT26A | RT13A | p66: K395A and E396A | expression failed | |
| RT27A | RT13A | p66: K527A, E529A, and K530A | expression failed | |
| RT28A | RT13A | p66: E449A | | RT28A/CL32543 a = 95.39 b = 96.39 c = 527.85 |
| | | | | p522 3.8 Å |
| RT29A | RT13A | p66: E297A, E298A, and E300A | expression failed | |
| RT30A | RT13A | p66: R463A/Q464S/K465L/V466S/V467S | | 5.5 Å |
| RT31A | RT13A | p66: K461R, P468T, and N471D | | low resolution powder |
| RT34A | RT13A | p66: full length | | 6 Å |
| RT35A | RT13A | p66: C258Q | | RT35A/JLJ0135 a = 89.98 b = 127.12 c = 254.94 |
| | | | | β = 93.55 P21 3.5 Å |
| RT52A | RT24A | p66: C258Q | | RT52A/TMC278 a = 163.37 b = 73.25 |
| | | | | c = 110.07 β = 100.84 C2 1.8 Å |
| | | | | RT52A/KMMP05 a = 183.56 b = 73.23 |
| | | | | c = 107.74 β = 100.15 C2 2.5 Å |
| | | | | RT52A/unliganded a = 235.4 b = 72.69 |
| | | | | c = 84.95 β = 104.87 C2 2.7 Å |
| RT52B | RT52A | P51: delta 447 | | RT52B/CL32543 a = 226.21 b = 69.06 |
| | | | | c = 103.77 β = 105.64 C2 2.8 Å |
| RT51A | RT52A | p66: L100I and K103N | | RT51A/TMC278 a = 184.15 b = 72.83 |
| | | | | c = 113.04 β = 101.42 C2 2.2 Å |
| RT55A | RT52A | p66: K103N and Y181C | | RT55A/TMC278 a = 182.84 b = 73.18 |
| | | | | c = 108.82 β = 100.79 C2 2.3 Å |
| RT61A | RT52A | p66: full length 560 | | no crystals |
| RT62A | RT52A | p51: no HRV 3c cleavage site but hexahis-tag on N- | | 3.8 Å |
| | | terminus | | |
| RT63A | RT52A | p51: same as RT1A | | no crystals |
| RT66A | RT52A | p66: I135A/N136A/E138A | | RT66A/JLJ0135 a = 89.98 b = 127.12 |
| | | | | c = 277.35 p212121 2.6 Å |
| RT67A | RT52A | p51: N-terminal deletion of PI5 | | no crystals |
| RT68A | RT52A | p66: delta 552 | | RT68A/JLJ0135 a = 162.30 b = 78.76 |
| | | | | c = 109.28 β = 100.18 C2 2.5 Å |
| RT69A | RT22A | p66: C258Q | | RT68A/LG-blue a = 184.01 b = 72.04 |
| | | | | c = 109.33 β = 104.38 C2 1.0 Å |
| RT70A | RT23A | p66: C258Q | | 7.0 Å |
| RT71A | RT28A | p66: C258Q | | 6.0 Å |
| RT72A | RT30A | p66: C258Q | | 6.0 Å |
| RT73A | RT52A | p66: K201A/E203A/E204A | | RT73A/JLJ0135 a = 90.81 b = 247.97 |
| | | | | c = 134.32 C222 3.6 Å |
| RT75A | RT52A | p66: E449A | | 3.8 Å |
| RT76A | RT52A | p66: R463A/Q464S/K465L/V466S/V467S | | no crystals |

TABLE 4
Crystallization Trial Kit developed for mutant RTs.

A1. 50 mM bis-tris propane, pH 6.8 100 mM ammonium sulfate,
10% v/v glycerol, 8% w/v PEG8000
A2. 50 mM bis-tris propane, pH 6.8 100 mM ammonium sulfate,
10% v/v glycerol, 9% w/v PEG8000
A3. 50 mM bis-tris propane, pH 6.8 100 mM ammonium sulfate,
10% v/v glycerol, 10% w/v PEG8000
A4. 50 mM bis-tris propane, pH 6.8 100 mM ammonium sulfate,
10% v/v glycerol, 11% w/v PEG8000
A5. 50 mM bis-tris propane, pH 6.8 100 mM ammonium sulfate,
10% v/v glycerol, 12% w/v PEG8000
A6. 50 mM bis-tris propane, pH 6.4 100 mM ammonium sulfate,
5% w/v sucrose, 5% v/v glycerol, 10% w/v PEG 8000. 20 mM
MgCl2
B1. 33% saturated ammonium sulfate, 100 mM sodium phosphate, pH 6.8
B2. 34% saturated ammonium sulfate, 100 mM sodium phosphate, pH 6.8
B3. 35% saturated ammonium sulfate, 100 mM sodium phosphate, pH 6.8
B4. 1.4M ammonium sulfate, 50 mM HEPES pH 7.2, 5 mM MgCl2,
300 mM KCl
B5. 6% w/v PEG3400, 24.35 mM citric acid, 51.5 mM Na₂HPO₄, pH 5.0
B6. 7% w/v PEG3400, 24.35 mM citric acid, 51.5 mM Na₂HPO₄, pH 5.0
C1. 8% w/v PEG3400, 24.35 mM citric acid, 51.5 mM Na₂HPO₄, pH 5.0
C2. 9% w/v PEG3400, 24.35 mM citric acid, 51.5 mM Na₂HPO₄, pH 5.0
C3. 10% w/v PEG3400, 24.35 mM citric acid, 51.5 mM Na₂HPO₄, pH 5.0
C4. 50 mM imidazole, pH 6.4, 100 mM ammonium sulfate, 15 mM
MgSO4, 10 mM Spermine, 10% w/v PEG 8000
C5. 50 mM imidazole, pH 6.4, 100 mM ammonium sulfate, 15 mM
MgSO4, 10 mM Spermine, 11% w/v PEG 8000
C6. 50 mM imidazole, pH 6.4, 100 mM ammonium sulfate, 15 mM
MgSO4, 10 mM Spermine, 12% w/v PEG 8000

The first round of mutagenesis/crystallization produced constructs RT1-10. None of these proteins produced crystals diffracting to beyond 10 Å resolution. The termini were then optimized for crystallization based on the notion that trimming the termini to residues visible in the electron density. The hexahistidine (6×His) purification tag on the C terminus of p51 was repositioned to the N and C termini of p66 and p51 in constructs RT12-14. Expression results showed that RT13A with a N terminal HRV14 3C cleavable 6×His-tag gave the highest yield of monodispersed protein, as measured by dynamic light scattering. The use of HRV14 3C protease to remove the 6×His-tag post-purification resulted in a N terminus with only an extra glycine (from the proteolytic cleavage site) compared to the natural terminus of the protein. The C terminus of p66 was terminated at residue 555 based on tandem mass spectroscopy results of RT crystals, indicating the last five residues to be proteolytically cleaved. The C terminus of p51 was truncated at 428 based on the indicated importance of this terminus in crystallization. RT13A was then used as the template in a third round of mutagenesis/crystallization.

3. Third Round of Mutagenesis/Crystallization

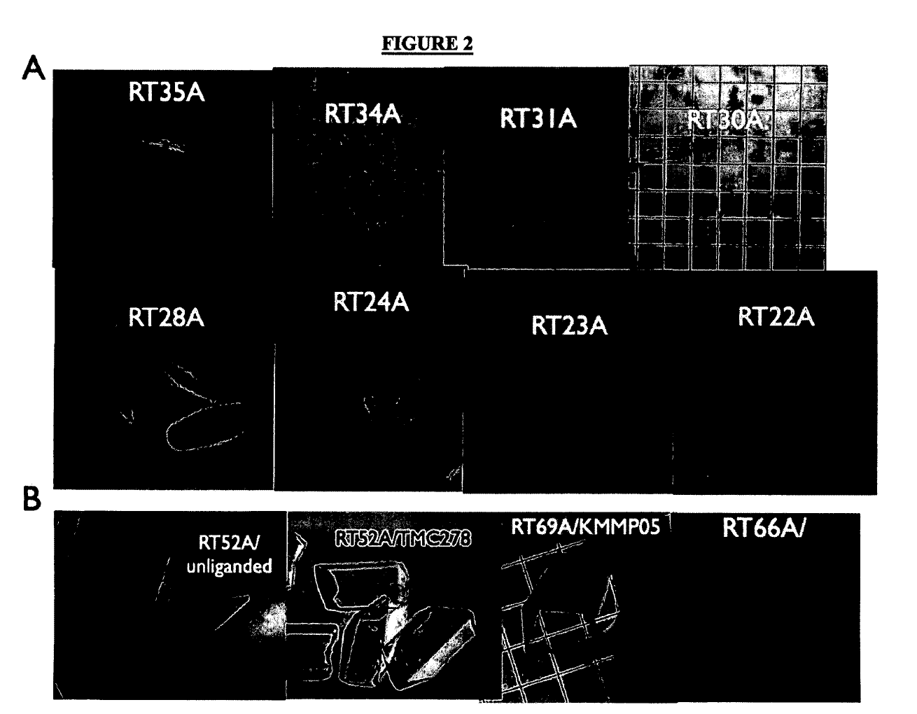

Constructs RT21-35 were then produced and crystallized unliganded and complexed with the NNRTIs CL32543 and TMC278. The new termini allowed for superior diffraction in several of the third round mutants compared to the first round of mutants. TMC278 co-crystals diffracted X-rays to higher resolution with RT24A than had been achieved with any previous RT construct. The diffraction resolution reached 3.3 Å but was very anisotropic and twinned which did not allow for structure determination. RT22A contains a PCR serendipitous mutation F160S but was used for crystallization as accidental mutations have historically been a source of improved crystallization (Braig et al., 1994, Pautsch et al., 1999). FIG. 2A shows the different crystal forms from the third round mutants.

A surprising result, where Q258C-RT which had been cross-linked to a RNA/DNA substrate but then had lost its substrate and crystallized unliganded with diffraction to 2.5 Å resolution, gave an important clue as to how to proceed. Crystals of Q258C-RT without a crosslinked substrate had never diffracted to better than 4 Å before, and the importance of the Q258C mutation was then considered. It was decided to revert residue 258 to glutamine in the fourth round of mutagenesis, which focused on RT24A, the construct that gave crystals with the highest resolution diffraction. It was also hypothesized that the anisotropy and twinning, of the RT24A/TMC278 crystal's diffraction, was originating with the TMC278's ability to wiggle and jiggle in evasion of the resistance mutations. In order to limit TMC278's flexibility in the NNRTI-binding pocket, two NNRTI resistance mutants were designed. RT51A contains mutations L100I and K103N while RT55A encodes K103N and Y181C. Both of the NNRTI double mutants are clinically significant, develop high resistance to NNRTIs, yet marginally resist inhibition by TMC278 with respective EC₅₀s 2.70 and 1.70 nM compared to 0.51 nM with wild-type RT (de Bethune et al. online poster 2005).

4. Diffraction of RT52A Crystals and its Derivatives RT52A, which is mutant RT24A without the Q258C mutation, was found to crystallize quickly (hours to days) and when complexed with NNRTIs could give high-resolution diffraction. Table 5 displays the data sets collected with RT52A, RT51A, and RT55A. The resolution of many of the NNRTI complexed data sets is without precedence for RT. RT52A/NNRTI crystals have symmetry of space group C2 with approximate cell parameters a=160-165, b=71-74, c=107-114 Å, □=□=90 and □=99-103°. This unit cell is novel when compared with all crystal structures of HIV-1 RT in the Protein Data Bank (Berman et al., 2000). Impressively, mutagenesis of the C terminus of p51 to delta 447 (which is present in 1B1) alone changes the unit cell to that seen with 1B1 complexed with NNRTIs, but with a loss in diffraction to 2.7 Å resolution (RT52B in Table 5).

TABLE 5
Collected X-ray diffraction datasets of RT with inhibitors.

| | | Ligand | | |
|---|---|---|---|---|
| Protein | Ligand | type | Resolution | Collected |
| RT52A | unliganded | | 3.2 | CHESS |
| RT52A | TMC278 | NNRTI | 1.8 | CHESS |
| RT51A | TMC278 | NNRTI | 2.9 | CHESS |
| RT55A | TMC278 | NNRTI | 2.1 | APS |
| RT52A | CL32543 | NNRTI | 2.1 | CHESS |
| RT52B | CL32543 | NNRTI | 2.7 | CHESS |
| RT55A | CL32543 | NNRTI | 3.0 | CHESS |
| RT52A | ADAM-II-CI | NNRTI | 2.3 | CHESS |
| RT52A | D372B | NNRTI | 2.4 | APS |
| RT52A | ADAM-II-CN | NNRTI | 2.55 | CHESS |
| RT52A | JLJ135 | NNRTI | 1.9 | CHESS |
| RT51A | JLJ135 | NNRTI | 2.5 | APS |
| RT55A | JLJ135 | NNRTI | 2.2 | CHESS |
| RT52A | TMC125 | NNRTI | 3.3 | CHESS |
| RT52A | TMC120 | NNRTI | 2.4 | CHESS |
| RT52A | NSC727717 | RNHI | 3.4 | APS |
| RT52A | NSC727448 | RNHI | 3.2 | APS |
| RT52A | KMMP02 | RNHI | 29 | BNL |
| RT52A | KMMP05 | RNHI | 2.5 | CHESS |
| RT69A | NSC727447 | RNHI | 1.9 | CHESS |
| RT69A | KMMP05 | RNHI | 1.85 | CHESS |
| RT69A | CL32543 | NNRTI | 3.0 | CHESS |

5. Enzymatic Assays of RT52A

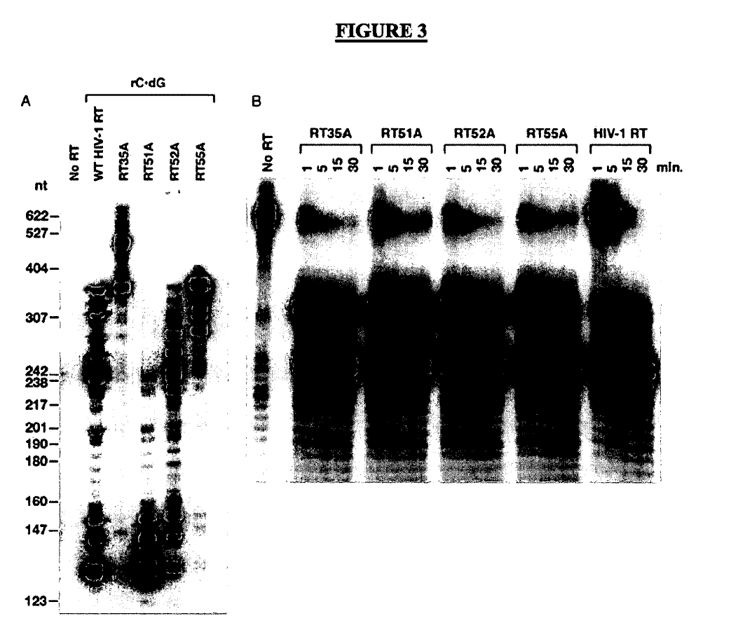

Proteins RT35A, RT51A, RT52A, and RT55A were tested for DNA-dependent DNA polymerase processivity and RNase H activity. FIG. 3A shows that RT52A has similar processivity as WT HIV-1 RT (RT co-expressed with HIV-1 protease), with RT51A having a diminished processivity and RT55A an increase. These results show that mutations K172A/K173A do not cause dramatic changes in the polymerase activity of RT. RT35A does not contain the lysine patch mutation and appears to have significantly increased processivity when compared to WT. The cause of this increased processivity is not clear, but it appears that the lysine patch mutation (K172A/K173A) causes a shift back to the processivity of the WT RT. Each of the mutants has similar RNase H activities (FIG. 3B).

6. RT52A Structure

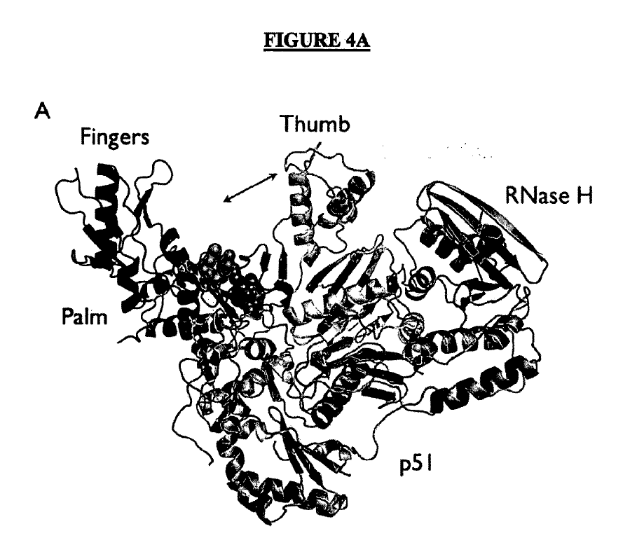

The electron density of TMC278 with RT52A is shown in FIG. 4. The crystal structures of TMC278 with and without NNRTI-resistance mutations unambiguously verifies the mechanism of inhibition when normally very effective resistance mutations are present.

7. RT52A Limitations

RT52A and its derivatives were very successful for NNRTI structure determination but did not achieve the same quality of diffraction for RNase H inhibitors (RNHIs) or when unliganded. For the fifth round of mutagenesis constructs were made to test the importance of each of the changes made to produce RT52A, and constructs that gave new crystal forms in round three were updated with the C258Q reversion and tested with RNHIs as well as NNRTIs to find a superior construct for RNase H studies. Constructs RT66A-RT69A were designed based on the electron density seen in RT52A structures. The mature RT52A termini were found to all be essential for diffraction, and mutation of three residues at a crystal contact 1135A/N136A/E138A were found to create a new crystal form.

Impressively, the construct RT69A produced crystals that gave high-resolution diffraction with two RNHIs while giving only 3.0 Å resolution diffraction with NNRTIs (Table 5). RT69A contains the accidental mutation F160S which is required for the crystal's improved diffraction. RT69A produces crystals within days but on average does not crystallize as quickly as RT52A. Thermal stability assays using circular dichroism have not shown significant changes in the stability of the mutants that would lead to the observed improvement in diffraction quality (Table 6).

Tm is the apparent melting temperature of the protein. RT samples were diluted in 2 mM HEPES (pH 8.2) and 75 mM NaCl to a final concentration of 0.12 mg/ml (0.3 ml total volume) and centrifuged at 15,000 g for 2 minutes before measurements. The thermal stability assay was performed starting at 4° C. and increased in 0.2° C. increments to 70° C. with 5 second measurements at 222 nm taken at each increment. The rate of temperature increase was 4.5° per 10 minutes. Thermal melting was not reversible.

TABLE 6
Thermal stability as measured by circular dichroism of RT
mutants.

| | | Tm Standard |
|---|---|---|
| Protein | Apparent Tm | Error |
| RT52A | 51.4 | 0.02 |
| RT69A | 51.4 | 0.03 |
| RT66A | 51.8 | 0.04 |
| RT35A | 51.2 | 0.03 |
| IBI | 52.4 | 0.03 |

### Discussion

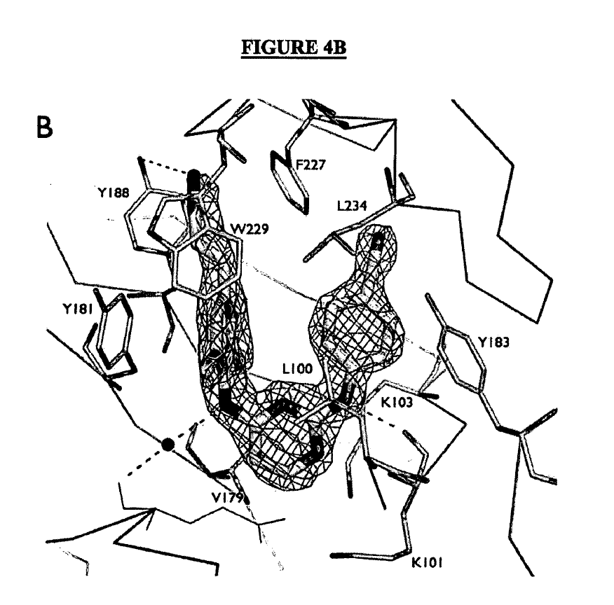

A mutagenesis/expression/purification system was created to allow for rapid testing of RT variants engineered for crystallization. The location of the 48 residues that were mutated is shown in FIG. 5. The distribution of the mutations was chosen to give the greatest variation to crystallization.

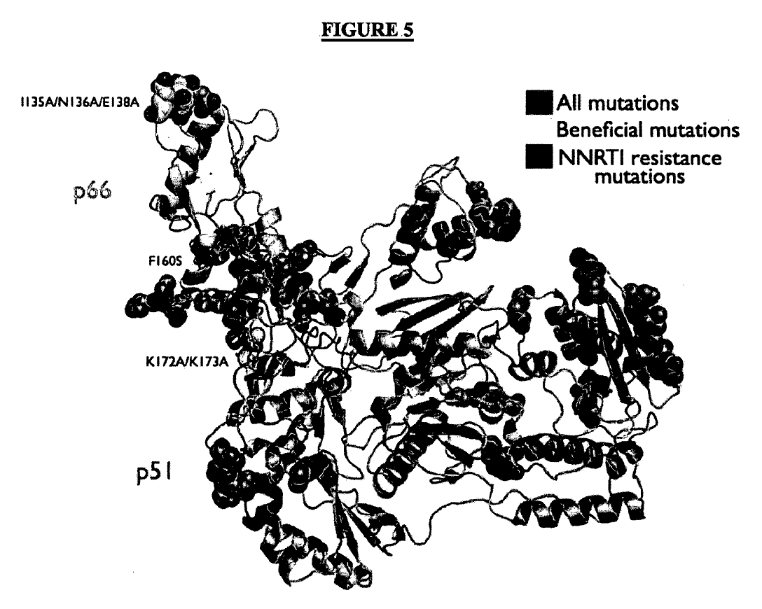

The exact mechanism of the improvement in crystallization has not been fully identified. It is clear from testing various mutants with reversions of the changes to Q258C-RT constituting RT52A that each of the mutations is required. Most of the reversions either caused a loss in crystallization or diminished diffraction quality. The Q258C mutation causes a change in space group to P6₂22 and a loss in diffraction quality while reverting the C terminus of p51 to delta 447 (the proteolytic cleavage site in 1B1 RT) causes the unit cell to be similar to that seen with 1B1 RT. Examination of the RT52A/NNRTI electron density shows that the crystal packing does not allow for extension of the p51 C terminus far beyond 428. The mutations K172A/K173A are near the symmetry-related p51 N terminus in the crystal lattice; however, the N terminus of p51 is disordered and probably not forming a crystal contact. The importance of the lysine patch mutation has been experimentally verified but the mechanism of its importance is not clear. FIG. 6 demonstrates the dramatic change in crystal contacts from 1131/NNRTI crystals to RT52A/NNRTI and RT69A/RNHI—a considerable increase in the number of RT regions involved in crystal contacts can be seen. The similarity in crystal contacts with RT52A and RT69A may indicate a possible mechanism for the improved crystallization. If the mutations bias a conformation without affecting the activities of the protein, then this conformation may be responsible for the improved diffraction quality. Thermal stability assays using circular dichroism have not shown significant changes in the stability of the mutants that would lead to the observed improvement in diffraction quality (Table 6).

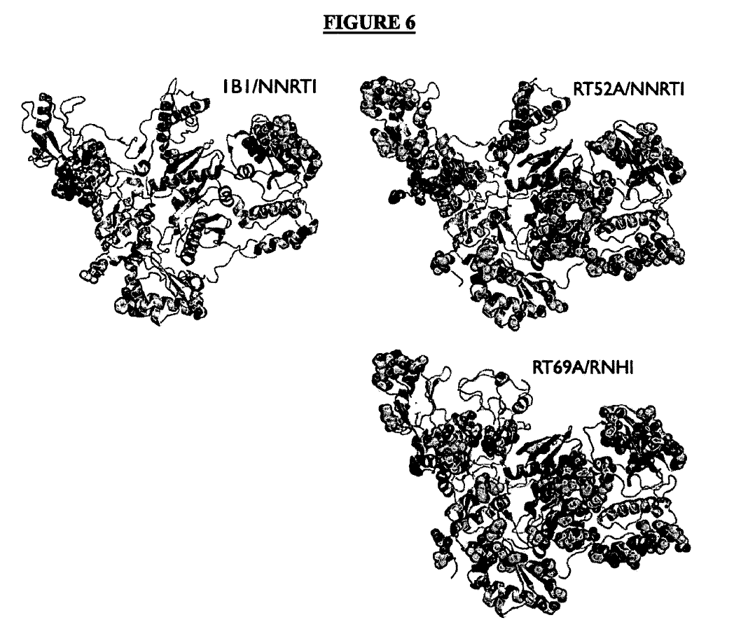

Protein engineering for crystallization, when prior structural knowledge isn't available, is primarily based on reducing disorder. The disorder can be in the form of long side chains, non-organized termini, flexible linkers, and other regions of high thermal energy. Recombinant technology adds another tool that can be used with purification technology to increase the homogeneity of a protein sample (decrease the disorder). When prior crystal structure knowledge exists it is possible to add an additional form of engineering in which known crystal contacts are enhanced or disrupted. Crystal contact disruption by mutagenesis can be shown through this and other work to be a very powerful technique for finding new crystal forms (Camara-Artigas et al., 2001, Charron et al., 2002, Honegger et al., 2005, Johnson et al., 2003, and Oubridge et al., 1995). FIG. 7 summarizes the X-ray diffraction resolution of crystals for each of the mutants tested.

A flexible protein like RT exists in many conformations in solution and can become relatively homogenous by the addition of a ligand, which may favor the stability of a single conformation or a subset of conformations. Different types of ligands induce different RT conformations and therefore different crystal forms. In the process of protein engineering for crystallization, it became clear that the engineering must be ligand type specific. The ligand specificity of crystallization has led to protein engineering being applied to other types of RT complexes that have been resistant to structural studies in the past. RT69A is the first of the successful constructs tested with ligand specificity in mind. Unfortunately, the mutation F160S affects a residue involved directly with nucleotide binding, Y115. RT69A may not be the optimal construct for studying RNHIs, but it does show the utility of this approach. Further work with current constructs as well as further mutagenesis is being carried out to study currently intractable RT complexes.

Thus the present invention identified a RT mutant which gave diffraction quality crystals in the presence of TMC278. The superior crystallizability and diffraction quality allowed by crystal engineering shows the usefulness of a systematic reiterative mutagenesis approach for crystallization of important drug targets. This success has led to the new ability of doing high-throughput crystallization of RT with NNRTIs. It is now possible to produce high-resolution diffraction within days of starting crystal trials with new NNRTIs. This provides, therefore, the long-needed, effective method for structure-based drug design through drug candidate co-crystallization studies as well as fragment screening (Hartshorn et al., 2005).

### Example 2

### Structural Studies of Engineered RT with the Potent NNRTI TMC278

1. Increased Order in the Polymerase Region of p66 Permits Higher Resolution Crystal Structures

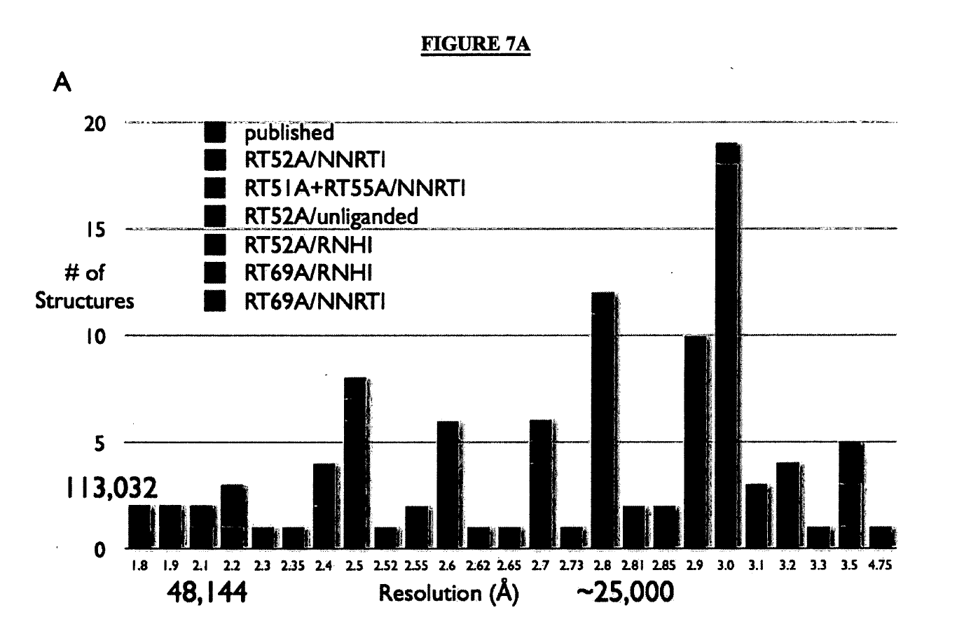

Crystals of wild-type RT/TMC278 had never diffracted to better than 8 Å after 5 years and thousands of crystallization experiments. The provided crystals of RT52A/TMC278 diffracted to better than 1.8 Å resolution. Table 7 and FIG. 8 show the statistical quality of structures of RT52A/TMC278. The new crystal form of RT52A/TMC278 is altered in many ways from wild-type crystals, though both use C2 space group symmetry. As described in Table 7B, the unit cell dimensions have decreased with a 9% decrease in solvent content. The smaller unit cell reflects the tighter packing of RT.

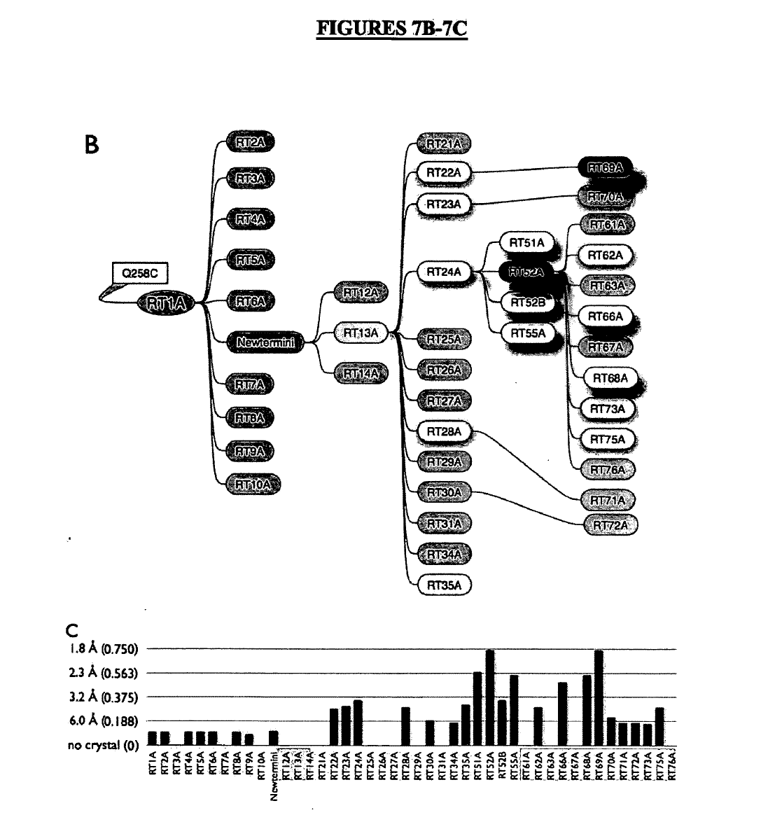

In the new crystal form the p66 thumb and finger subdomains are constrained in the crystal lattice resulting in increased order in the crystals. The increased order produces higher resolution diffraction by X-rays. As depicted in FIG. 9, the p66 fingers subdomain is bounded by the RNaseH domain and p51 subdomain of two different symmetry-related molecules. Tighter packing restricts the structural heterogeneity of the cleft-open form of RT, and therefore the crystals of RT52A/TMC278 are able to diffract to a surprisingly high 1.8 Å resolution.

2. Validity of Engineered RT Structures

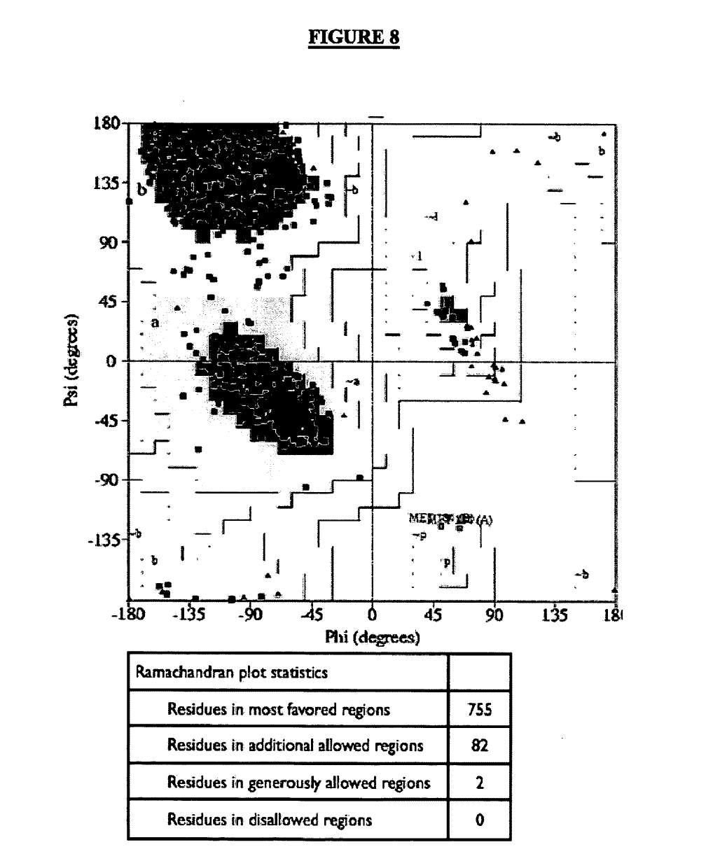

The structure of RT52A in comparison to other NNRTI/RT structures is shown in FIG. 10. The RT52A/TMC278 structure has a RMSD of 2.44 Å compared to a non-engineered RT structure with NNRTI Janssen-R129385 (Das et al., 2004). A large shift in a p66 palm subdomain loop (near residue 222) of 6.6 Å is responsible for 0.2 Å of the RMSD between the TMC278 and R129385. The shift of the palm subdomain loop is seen in other NNRTI structures in the Protein Data Bank and the RMSD of the engineered RT is similar to what it is seen between structures from

TABLE 7
Data collection and refinement statistics of RT with TMC278
A

| | | [symbol] | [symbol] |
|---|---|---|---|
| | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | | |
| [symbol] | [symbol] | [symbol] | [symbol] |
| [symbol] | [symbol] | [symbol] | [symbol] |
| | [symbol] | [symbol] | [symbol] |

[symbol]

| [symbol] | [symbol] | [symbol] | [symbol] |
|---|---|---|---|
| | | [symbol] | |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | | |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |

B

| | [symbol] | [symbol] |
|---|---|---|
| [symbol] | [symbol] | [symbol] |
| [symbol] | [symbol] | [symbol] |
| | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |

[symbol]

indicates data missing or illegible when filed

different strains of HIV-1 (˜3.0 Å). Analyses of the secondary structure of the TMC278 and R129385 crystals shows small differences (FIG. 10B). The improved electron density due to the new crystal contacts allows for a clearer delineation of secondary structure in these important regions.

3. Binding of TMC278 to the WT NNIBP

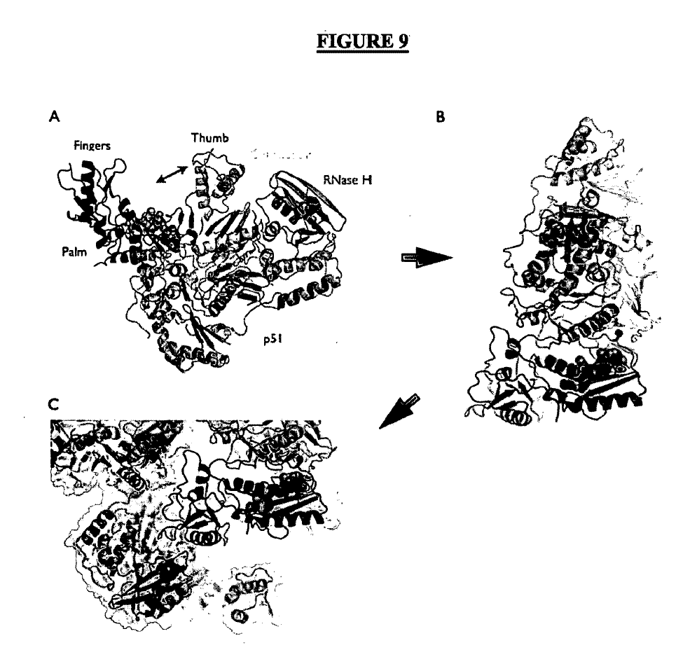

The high-resolution electron density maps precisely define the position of each non-hydrogen atom of the inhibitor (FIG. 11). The mode of binding is the “horseshoe” mode that has been seen for other DAPY compounds (Das et al., 2004). The “wings” of the NNRTI make π-π stacking interactions with Tyr181, Tyr188, and Tyr318.

A distinguishing feature of TMC278 to the other DAPY compounds is a cyanovinyl on “Wing 1.” The cyanovinyl is positioned in a hydrophobic tunnel composed of the is sidechains of Tyr188, Phe227, Trp229, and Leu234. The hydrophobic tunnel opens toward the nucleic acid binding cleft near the polymerase active site. The interaction of the cyanovinyl group and the tunnel explains the improved potency of TMC278 compared to other DAPY NNRTIs. The torsional flexibility of the cyanovinyl group should allow TMC278 to bind RT with mutations in the tunnel, such as the Tyr188Leu mutation.

4. Binding of TMC278 to Leu100Ile-Lys103Asn Double Mutant

TMC278 overcomes all resistance mutations that it has been tested against. The mutant that had the greatest effect on the EC₅₀ (the 50% effective concentration) was the double mutant Leu100Ile/Lys103Asn. The EC₅₀ of the double mutant was 7 nM versus 0.4 nM for wild-type RT. The crystal structure was determined at 2.9 Å resolution to elucidate the mechanism that TMC278 uses to overcome this very potent resistance double mutation. The RMSD of RT52A/TMC278 with the 2.9 Å Leu100Ile/Lys103Asn structure is 0.82 Å.

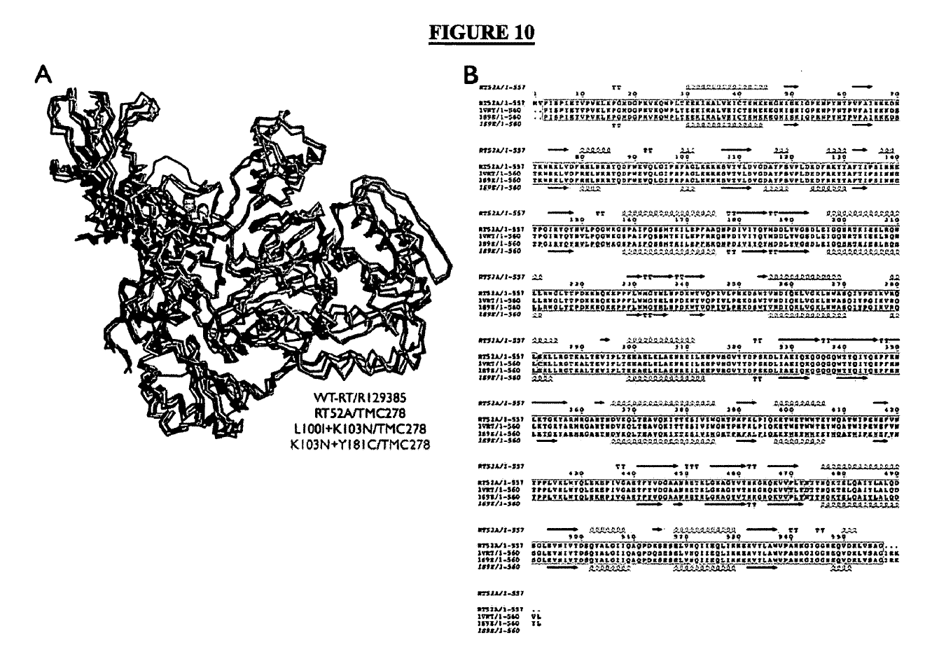

FIG. 12 shows the clear electron density defining the binding of TMC278 to the mutant RT. One of the interesting features of the structure is that TMC278 develops a hydrogen bond with Asn103 instead of the hydrogen bond with the Ile101 main-chain carbonyl. The interaction with Asn103 should help overcome the resistance of this mutation due to TMC278 disrupting the hydrogen bond network it normally forms in the unliganded structure. The Leu100Ile mutation causes a steric hindrance in NNIBP. TMC278 “wiggles” by altering its torsional angles and “jiggles” by translating 1.3 Å in the pocketto adjust to the steric hindrance. By being able to bind in the NNIBP in multiple conformations, TMC278 is able to inhibit multiple variations of the NNIBP. The wiggling and jiggling phenomenon was, first described from a single mutant structure of RT/TMC125 (Das et al., 2004). This is the first study to directly show multiple conformations of the same inhibitor with different RT mutants.

5. Binding of TMC278 to Lys103Asn/Tyr181Cys Double Mutant

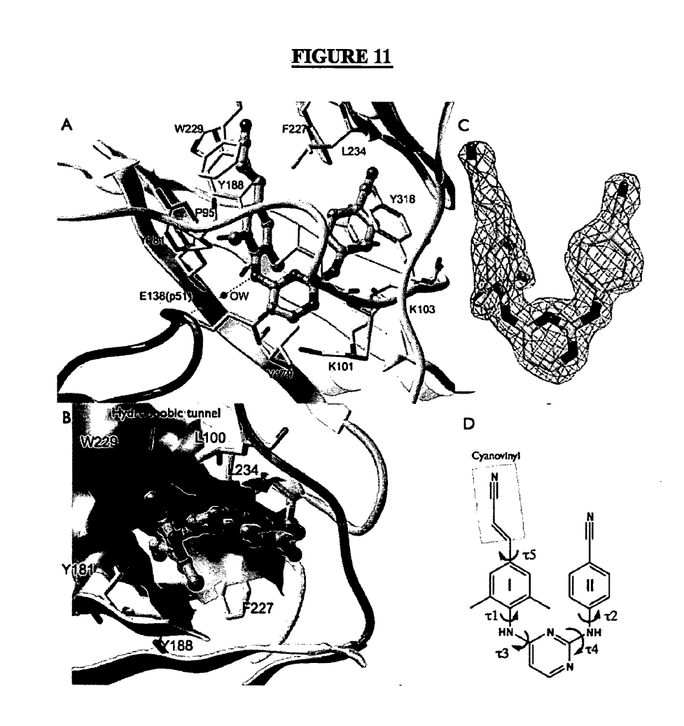

Lys103Asn and Tyr181Cys mutants were present in more than 10% of the patients failing retroviral therapy in one study (Cheung et al., 2004). The Lys103Asn/Tyr181Cys double mutant is resistant to all available NNRTIs, but TMC278 has an EC₅₀ of 1.0 nM against it (Guillemont et al., 2005; Janssen et al, 2005). To show how TMC278 avoids the resistance mutations we solved a 2.1 Å resolution structure of it with the double mutant Lys103Asn/Tyr181Cys. The RMSD of RT52A/TMC278 with Lys103Asn/Tyr181Cys structure is 0.61 Å. FIG. 13 depicts an overlay of RT52A/TMC278 and Lys103Asn/Tyr181Cys with TMC278. Similar to the other double mutant, TMC278 makes a hydrogen bond with Asn103. Loss of Tyr181 permits a shift in the Tyr183, which partially compensates for the lost interaction with Tyr181. This latter observation is especially interesting—Tyr183 is part of the “YMDD motif,” which is highly conserved in all HIV-1, HIV-2, and SIV RTs, and even present in HBV polymerase. The cyanovinyl moiety of TMC278 makes a favorable interaction with the aromatic side chain of Y183, essentially “recruiting” a portion of the polymerase active site to help in binding the NNRTI to compensate for loss of stabilizing interactions caused by the cysteine replacement of Tyr181.

6. Summary of Torsional Flexibility

Table 8 summarizes the torsional flexibility of TMC278 with the two double mutants structurally determined in this study compared to the wild-type NNIBP protein. It is clear from the change in angles that the torsional flexibility of the cyanovinyl and “Wing 1” allows TMC278 to overcome the resistance mutation Leu100Ile/Lys103Asn. This is the first study to directly demonstrate strategic flexibility in a series of mutants with the same inhibitor, providing a dramatic confirmation that wiggling and jiggling of an inhibitor can permit activity against a broad range of drug-resistant variants of a target such as HIV-1 RT.

TABLE 8
TMC278 torsional angles.

| | t1 | t2 | t3 | t4 | t5 |
|---|---|---|---|---|---|
| RT52/TMC278 | −94 | 17 | −12 | −9 | −45 |
| K103N-Y181C/ | −96 | 17 | −7 | −6 | −50 |
| TMC278 | | | | | |
| L100I-K103N/ | −115 | −2 | −6 | 10 | −4 |
| TMC278 | | | | | |

### Example 3

### Crystal Engineering of HIV-1 Reverse Transcriptase for High-Throughput Crystallography

1. Expression Vector and Mutation Construction

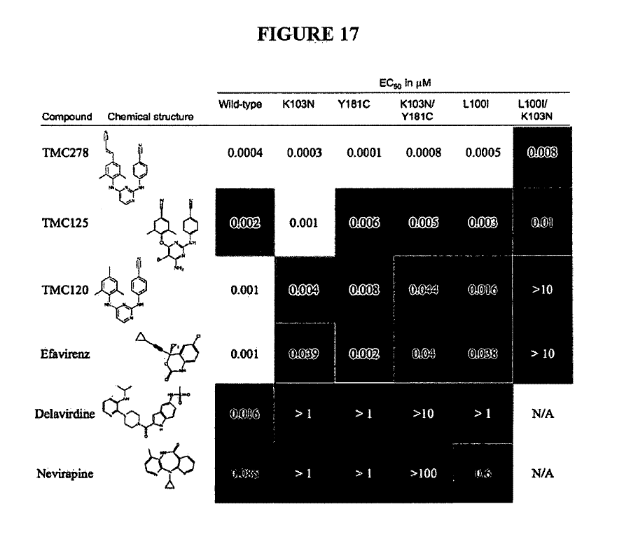

The RT coding DNA from the Q258C-RT construct (Sarafianos et al., 2003) was ligation independent cloned (LIC), with all vector-encoded amino acid sequence eliminated by restriction digestion post-LIC, into pCDF-2 Ek/LIC with the LIC Duet™ Minimal Adaptor (Novagen) according to manufacturer's recommendations. The RT-encoding dual expression vector is designated pRT1. Mutagenesis was completed using MOE-LIC. See FIG. 17A for the location and pairing of the primers on pRT1. The methylated and non-methylated primers are listed in Table 11.

To minimize false positive colonies the vector was restriction digested with the appropriate restriction enzymes to remove the ORF protein-coding DNA that was to be replaced (NcoI and SacI for ORF-1 or NdeI and XhoI for ORF-2). For ORF-2, 3 μl of vector (250 ng/μl) was digested in a 20 μl volume with 1 μl NdeI (20,000 units/ml) and 1 μl XhoI (20,000 units/ml) for one hour at 37° C. with NEBuffer2 (New England Biolabs). For p66, mutagenesis overlap extension PCR was performed using mutated overlap segments with the 2′-O-methylated primers to amplify the full insert with PfuUltra™ II Fusion HS DNA Polymerase (Stratagene). A typical overlap extension PCR was performed with 1 μl of each template, 1 μl (20 pmols) of each primer, 39 μl water, 1 μl (25 mM each) dNTPs, 5 μl 10× PfuUltra buffer, and 1 μl PfuUltra™ H Fusion HS DNA Polymerase (Stratagene). The PCR program is listed: 3 min at 95° C.; followed by 5 cycles of 1 min at 95° C., 1 min at 50° C., and 30 s at 72° C.; 30 cycles of 30 s at 95° C., 30 s at 53° C., and 45 s at 72° C.; ending with a final extension step of 10 min at 72° C. The digested vector is [was?would be] amplified in a separate reaction tube with complementary methylated primers.

The PCR products were then gel purified, and 0.04 pmols of vector and insert were mixed at a 1:1 insert to vector molar ratio in a buffer containing 25 mM Tris pH 8.0, 5 mM MgCl₂, 0.025 mg/ml BSA, and 2.5 mM DTT in a 20 μl volume. The mixture was heated to 70° C. and cooled slowly over 2 h in a water bath. Once cooled to ˜40° C., 1 μl of 25 mM EDTA was added and the mixture incubated at room temperature for 5 min before being desalted using a Centri-Sep column (Princeton Separations) or ethanol precipitation (Donahue et al., 2002). Five μl of desalted annealed DNA was added to electrocompetent NovaBlue cells (Novagen) and electroporated according to manufacturer's recommendations.

2. Expression and Purification of RT

pRT containing BL21-CodonPlus®-RIL cells were induced with 1 mM IPTG at an OD₆₀₀ of 0.9 followed by expression at 37° C. for three hours. Ni-NTA purification was performed according to the manufacturer's recommendations (Qiagen) with the following modifications: no added lysozyme, 600 mM NaCl in each of the standard buffers, 0.1% Triton X-100 added to the lysate and wash buffers, and a high-salt wash step performed with 1.2 M NaCl added to the standard wash buffer. After elution the HRV14 3C protease was added (1:100 ratio of protease:RT) and incubated at 4° C. overnight. Mono Q was performed as described (Clark et al., 1995). The RT was buffer exchanged and concentrated to 20 mg/ml in 10 mM Tris pH 8.0 and 75 mM NaCl. The concentrated RT was aliquoted and stored at −80° C. or placed at 4° C. for immediate crystallization.

3. Crystallization

The RT was screened unliganded, with a 2.5-fold molar excess of NNRTI, or with a 5-fold molar excess of RNHI using the hanging-drop vapor diffusion method. Depending on the number of samples being screened, EasyXtal DG-Tools (Qiagen) or Linbro Plates (Hampton Research) crystallization trays were used for screening. Based on visually identified crystal hits, further optimization was used. RT52A and RT69A crystals were produced in a matrix of 24 conditions from 9-12% PEG 8000, 50 mM imidazole pH 6.0-6.8, 10 mM spermine, 15 mM MgSO₄, and 100 mM ammonium sulfate. All successful crystallization experiments were performed at 4° C.

4. Data Collection

Crystals of RT52A were flash-cooled by immersion into liquid nitrogen after briefly dunking the crystal into cryoprotective solution containing well solution plus 27% ethylene glycol and the inhibitor at the same concentration as in the hanging drop. Best results were found when using MicroMounts (MiTeGen) for mounting the crystals. Data for screening and data set collection were obtained at the Cornell High Energy Synchrotron Source (CHESS) F1 and A1 beamlines, National Synchrotron Light Source (NSLS) beamlines X25 and X29, and Advanced Photon Source (APS) at Argonne National Laboratory (ANL), SER-CAT beamline 191D. The diffraction data were indexed, processed, scaled and merged using HKL2000 (Otwinowski et al., 1999). The resolution of the data was estimated using the last resolution shell values for completeness, R-merge, and the ratio of I to σ(I).

5. RT Activity Assays

The DDDP processivity assay was done as previously described (Boyer et al., 2002). The RNase H activity assay was performed as described (Boyer et al., 2004).

6. Results

Engineered RTs were mutagenized using the novel, flexible and cost effective method of the present invention, known as methylated overlap-extension ligation independent cloning (MOE-LIC). The new RT constructs faciliatates fast and high resolution structure determination that is enhancing the understanding of the enzyme's mechanisms and accelerating the design of improved drugs targeting RT.

The present Example used a co-expression system that allows subunit-specific mutagenesis at multiple positions and the addition of a purification tag on the C or N terminus of the subunit of choice for facile purification. In the initial co-expression construct, the p51 subunit consisted of 428 residues and a hexahistidine purification tag at the C terminus (Huang et al., 1998 and Sarafianos et al., 2003). The co-expression construct codes for the p66 Q258C mutant, which is used to produce homogenous nucleic-acid cross-linked samples for X-ray crystallographic studies. This plasmid facilitates expression, purification, and crystallization of multiple RT constructs in parallel.

To produce diffraction quality crystals of RT with TMC278, a crystal engineering technique was developed that employs an iterative high-throughput approach to create and test RT mutants for crystallization. The approach examines many RT mutants in parallel for cloning, expression, purification, and crystallization. Based on the quality of the X-ray diffraction from the crystals, the next round of mutagenesis uses the best construct from the previous round as a template and other information obtained from the experiment for optimization. It was thereby attempted to artificially evolve RT for improved crystallization in a cyclic process in which the fittest RT construct from the previous cycle is the parental template for the next cycle (FIG. 17).

### Co-Expression and Mutant Cloning

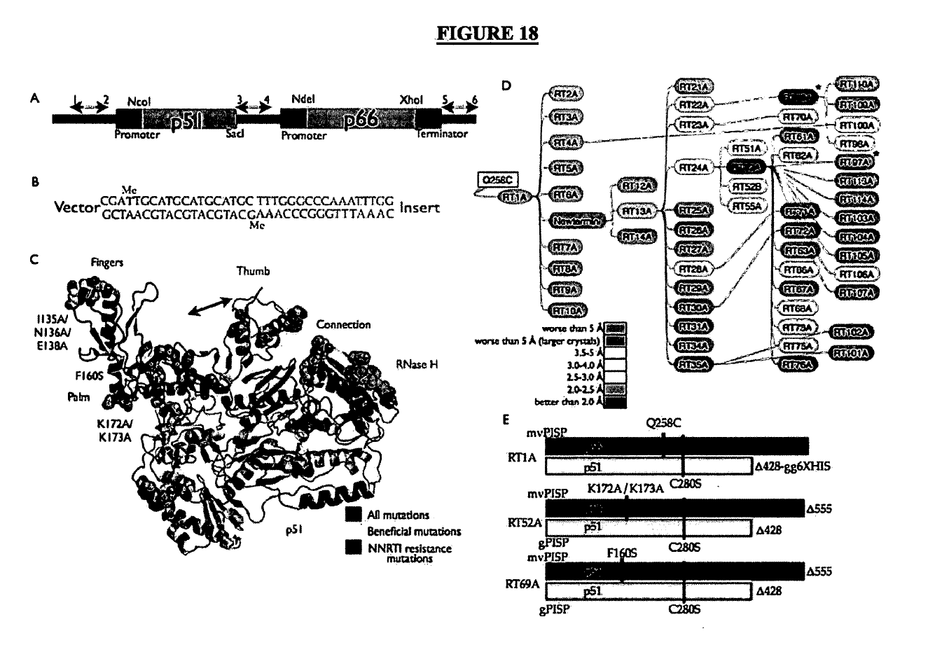

A modular co-expression system was chosen to allow high-throughput subunit-specific mutagenesis of RT (FIG. 18A). The system allowed for high expression yield (˜40 mg/liter) under standard expression conditions. In the expectation of creating many RT mutants, a rapid, high yield, and inexpensive mutagenesis system was sought. Donahue et al. (2002) proposed a ligation independent cloning technique, which uses terminator primers to create 12-15 nucleotide complementary overhangs on the insert and vector. The insert and vector are annealed and transformed into bacteria, thereby avoiding any post-PCR enzymatic steps. The terminating residue in the primer is a 2′-O-methylated nucleotide, which causes early termination of thermostable polymerases Taq or Pfu (FIG. 18B). There are two major limitations with this technique: 1) the 2′-O-methylated primers cost ˜$100 per pair and 2) the site of 2′-O-methylation has a 20% mutation rate.

A novel terminator primer technique for rapid mutagenesis of RT called methylated overlap-extension ligation independent cloning (MOE-LIC) was developed by the present invention. MOE-LIC uses overlap-extension mutagenesis (Ho et al., 1989) and terminator primers outside the open reading frame (ORF) to avoid unwanted mutagenesis of the coding or regulatory regions. Overlap-extension PCR provides the flexibility for inserting a completely new sequence or mutagenizing a previously constructed insert (Horton et al., 1989). For the co-expression system a total of three terminator primer pairs (FIG. 18A) were required at a cost of approximately $300, which could be used for over a thousand reactions with no additional enzyme cost besides the PCR polymerase. Error rates were found to be extremely low with one unintended mutation found per 30 mutants produced.

### Mutagenesis and Crystallization

A protein engineering methodology for the crystallization of RT was developed by combining several strategies as follows: 1) disrupt or enhance common crystal contacts in the existing crystal forms of RT; 2) remove high B-factor patches, primarily disordered termini in the parent C2 RT/NNRTI crystal form; 3) reduce surface entropy by mutagenesis of lysine and glutamic acid patches to alanine (for review Derewenda and Vekiloc, 2006); 4) use the wealth of information about multiple crystal forms of RT (e.g., sequence variations, different sets of crystal contacts, ordered/disordered regions, etc.); 5) avoid mutating conserved residues; and 6) use multiple iterative rounds of mutagenesis/crystallization to improve the X-ray diffraction quality (FIG. 17). FIG. 18C shows the location of the mutations that were made for crystallization trials (see Table 9 for a complete list of the 59 RT variants and the diffraction resolution of the crystals). Eighteen crystallization conditions, chosen from previously reported crystallographic studies of HIV-1 RT (Clark et al., 1995, Chan et al., 2001, Rodgers et al., 1995, Hogberg et al., 1999, and unpublished data), were used for the initial crystal screening of each RT variant (Supplemental Table 10). Crystallization of individual RT samples was attempted unliganded, with TMC278, and with other NNRTIs in parallel.

The first round of mutagenesis/crystallization produced constructs RT1-10 and crystals of RT/TMC278 complexes that diffracted to very poor resolution (FIG. 18D). Although none of the constructs produced improved X-ray diffraction quality, one construct where p66 is terminated at residue 555 produced larger crystals than those terminated at residue 560. In the next cycle, the termini for both the p66 and p51 subunits were optimized. Based on the notion that disordered termini residues hinder tight packing in the crystal lattice, any disordered residues at the termini, including purification tags, were removed prior to crystallization. The C termini were truncated at residue 555 for p66 and 428 for p51 based on knowledge from existing RT crystal forms. Of the three round two constructs, RT13A with a N-terminal HRV14 3C cleavable 6×His-tag gave the highest yield of monodisperse protein, as measured by dynamic light scattering, suggesting the sample as the optimal candidate for crystallization trials.

RT13A became the template for the third round of mutagenesis, resulting in constructs RT21-35. The crystals of RT24A/TMC278 complex diffracted X-rays to 3.3 Å resolution, which was the best achieved with TMC278) compared with any previous RT construct. The 3.3 Å diffraction dataset was anisotropic and produced multiple diffraction patterns, which did not permit obtaining a reliable complete data set necessary for structure determination. All the above constructs of RT had a p66 Q258C mutation that was used for cross-linking RT to nucleic acid (Huang et al., 1998 and Sarafianos et al., 2003). It was decided to revert residue 258 to glutamine in the fourth round of mutagenesis to reduce any disorder resulting from having a surface cysteine residue not crosslinked to nucleic acid.

New Crystal Form and High-Resolution Diffraction from RT52A/NNRTI Crystals

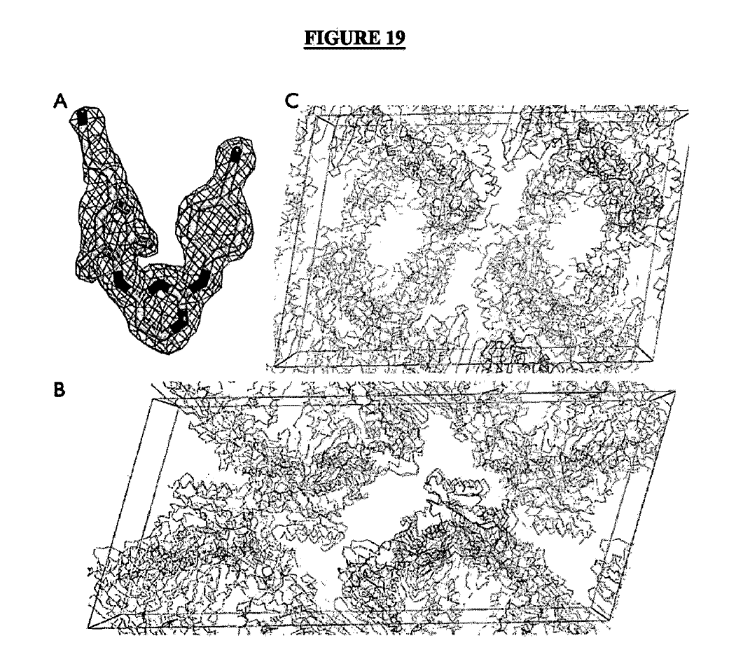

RT52A (FIG. 18E), which is RT24A with a C258Q reversion, when complexed with TMC278 and other NNRTIs could produce crystals within 1-3 days. The crystals of the RT52A/NNRTI complexes diffracted X-rays to high resolution (often better then 2.0 Å resolution). The quality of the 1.8 Å RT52A/TMC278 structure (Das et al., 2008) is evident from the high-resolution electron density map for the inhibitor shown in FIG. 19A. The structures of RT52A/NNRTI complexes revealed a new crystal form of RT. This new crystal form has preserved the symmetry of its parent crystal space group C2, but with distinctly different unit cell parameters and crystal contacts (FIG. 19B-C). Tighter crystal packing of RT52A molecules is evident from a 14% decrease in solvent content and a 19% decrease in unit cell volume compared to NNRTI complexed with non-engineered RT (construct designated 1B1) (Clark et al., 1995). There is also a near doubling in the number of residues involved in crystal packing (within 4.5 Å of each other), from 97 residues to 194, and the surface area involved in crystal contacts, from 1556 Å² to 2707 Å²(http://www.ebi.ac.uk/msd-srv/prot_int/pistart.html).

For 1B1-RT/NNRTI crystallization studies, RT was expressed as a single-chain p66 that produces a p51 chain via cleavage at residue 447 by a co-purifying bacterial protease, ultimately yielding p66/p51 heterodimer (Clark et al. 1995). Impressively, altering RT52A at the C terminus of p51 to terminate at 447 alone changes the unit cell to that seen with 1B1-RT complexed with NNRTIs, but with a significant drop in diffraction resolution to 2.7 Å (Table 9). Mutants were then constructed to test each of the changes made to produce RT52A, and each was found to be required for high-resolution X-ray diffraction (Table 9). These results provide clear evidence of the benefit of crystal engineering through RT mutations at multiple sites.

TABLE 9

| | | | Clone/ | |
|---|---|---|---|---|
| Construct | Template | Mutation | Express | Crystals/Diffraction |
| RT1A | | Paul Boyer's Q258C construct in | | 10 Å |
| | | pRT | | |
| RT2A | RT1A | p66: K172R | | 10 Å |
| RT3A | RT1A | p66: K512Q | | no crystals |
| RT4A | RT1A | p66: E28A | | 10 Å |
| RT5A | RT1A | p51: L92 and G93 removed | | 10 Å |
| RT6A | RT1A | p66: delta 3 N-terminus | | 10 Å |
| RT7A | RT1A | p66: delta 4 N-terminus | | no crystals |
| RT8A | RT1A | p51: delta 3 N-terminus | | 10 Å |
| RT9A | RT1A | p51: delta 4 N-terminus | | 12 Å |
| RT10A | RT1A | p51: delta 427 | | no crystals |
| Newterm | RT1A | p66: delta 555; p51: delta 447 | | 9 Å |
| ini | | | | |
| RT12A | RT1A | p66: N-terminus HRV3c-hexahistag | | |
| | | delta 555; p51: delta 428 | | |
| RT13A | RT1A | p51: N-terminus HRV3c-hexahistag | Best | crystals - no |
| | | delta 428; p66 delta 555 | expression | diffraction |
| | | | of | |
| | | | RT12A-14A | |
| RT14A | RT1A | p66: C-terminus hexahistag; p51: | | |
| | | delta 428 | | |
| RT21A | RT13A | p66: K223A and E224A | cloning | |
| | | | failed | |
| RT22A | RT13A | p66: F160S | | 4.0 Å |
| RT23A | RT13A | p66: K219A and K220A | | 3.7 Å |
| RT24A | RT13A | p66: K172A and K173A | | RT24A/TMC278 |
| | | | | a = 99.95 b = 99.95 |
| | | | | c = 515.469 p622 |
| | | | | 3.6 Å |
| | | | | RT24A/CL32543 |
| | | | | (NNRTI) a = 73.51 |
| | | | | b = 90.44 c = 126.17 |
| | | | | α = 105.39 |
| | | | | β = 94.73 γ = 110.37 |
| | | | | p1 3.3 Å |
| RT25A | RT13A | p66: K201A/E203A/E204A | cloning | |
| | | | failed | |
| RT26A | RT13A | p66: K395A and E396A | expression | |
| | | | failed | |
| RT27A | RT13A | p66: K527A, E529A, and K530A | expression | |
| | | | failed | |
| RT28A | RT13A | p66: E449A | | RT28A/CL32543 |
| | | | | (NNRTI) a = 96.39 |
| | | | | b = 96.39 c = 527.85 |
| | | | | p622 3.8 Å |
| RT29A | RT13A | p66: E297A, E298A, and E300A | expression | |
| | | | failed | |
| RT30A | RT13A | p66: R463A/Q464S/K465L/V466S/ | | 5.5 Å |
| | | V467S | | |
| RT31A | RT13A | p66: K461R, P468T, and N471D | | low resolution |
| | | | | powder |
| RT34A | RT13A | p66: full length | | 6 Å |
| RT35A | RT13A | p66: C258Q | | RT35A/JLJ0135 |
| | | | | (NNRTI) a = 89.98 |
| | | | | b = 127.12 |
| | | | | c = 254.94 β = 93.66 |
| | | | | P21 3.6 Å |
| RT52A | RT24A | p66: C258Q | | RT52A/TMC278 |
| | | | | a = 163.37 b = 73.26 |
| | | | | c = 110.07 |
| | | | | β = 100.84 C2 1.8 Å |
| | | | | RT52A/KMMP05 |
| | | | | (RNHI) a = 163.56 |
| | | | | b = 73.23 c = 107.74 |
| | | | | β = 100.15 C2 2.5 Å |
| | | | | RT52A/unliganded |
| | | | | a = 235.4 |
| | | | | b = 72.69 c = 94.95 |
| | | | | β = 104.87 C2 2.7 Å |
| RT52B | RT52A | P51: delta 447 | | RT52B/CL32543 |
| | | | | (NNRTI) a = 226.21 |
| | | | | b = 69.06 c = 103.77 |
| | | | | β = 105.64 C2 2.8 Å |
| RT51A | RT52A | p66: L100I and K103N | | RT51A/TMC278 |
| | | | | a = 164.15 b = 72.93 |
| | | | | c = 113.04 |
| | | | | β = 101.42 C2 2.2 Å |
| RT55A | RT52A | p66: K103N and Y181C | | RT55A/TMC278 |
| | | | | a = 162.84 b = 73.18 |
| | | | | c = 108.92 |
| | | | | β = 100.79 C2 2.3 Å |
| RT61A | RT52A | p66: full length to 560 | | no crystals |
| RT62A | RT52A | p51: no HRV 3c cleavage site but | | 3.8 Å |
| | | hexahis-tag on N-terminus | | |
| RT63A | RT52A | p51: same as RT1A | | no crystals |
| RT66A | RT52A | p66: I135A/N136A/E138A | | RT66A/JLJ0135 |
| | | | | (NNRTI) a = 89.98 |
| | | | | b = 127.12 |
| | | | | c = 277.35 |
| | | | | p212121 2.6 Å |
| RT67A | RT52A | p51: N-terminal deletion of PIS | | no crystals |
| RT68A | RT52A | p66: delta 552 | | RT68A/JLJ0135 |
| | | | | (NNRTI) a = 162.30 |
| | | | | b = 73.76 c = 109.28 |
| | | | | β = 100.18 C2 2.5 Å |
| [symbol] | [symbol] | [symbol] | [symbol] | [symbol] |
| RT70A | RT23A | p66: C258Q | | 7.0 Å |
| RT71A | RT28A | p66: C258Q | | 6.0 Å |
| RT72A | RT30A | p66: C258Q | | 6.0 Å |
| RT73A | RT52A | p66: K201A/E203A/E204A | | RT73A/JLJ0135 |
| | | | | (NNRTI) a = 90.81 |
| | | | | b = 247.97 |
| | | | | c = 134.32 C222 |
| | | | | 3.6 Å |
| RT75A | RT52A | p66: E449A | | 3.8 Å |
| RT76A | RT52A | p66: R463A/Q464S/K465L/V466S/ | | no crystals |
| | | V467S | | |
| RT97A | RT52A | p66: P468T/N471D | + | RT97A/unliganded |
| | | | | a = 164.02 |
| | | | | b = 71.05 |
| | | | | c = 108.73 |
| | | | | β = 104.5 C2 2.1 Å |
| | | | | But 1.5° |
| | | | | mosaicity |
| RT98A | RT69A | p66: P468T/N471D | + | RT98A/GMMP028 |
| | | | | (RNHI) |
| | | | | a = 143.37 b = 73.4 |
| | | | | c = 109.9 β = 91.5 |
| | | | | 2.6 Å |
| | | | | But 1.6° |
| | | | | mosaicity |
| RT100A | RT35A | p66: E28A | | RT100A/GMMP028 |
| | | | | (RNHI) 2.8 Å |
| RT101A | RT35A | p66: K43A/E44A/K49A | | RT101A/JLJ0135 |
| | | | | (NNRTI) a = 162 |
| | | | | b = 220 c = 109 |
| | | | | β = 100.4 C2 2.9 Å |
| RT102A | RT35A | p66: N348I | | RT102A/JLJ0135 |
| | | | | (NNRTI) a = 162 |
| | | | | b = 73.4 c = 108.9 |
| | | | | β = 100.4 C2 2.7 Å |
| | | | | RT102A/GMMP028 |
| | | | | (RNHI) |
| | | | | a = 164.39 |
| | | | | b = 72.20 |
| | | | | c = 109.11 |
| | | | | β = 104.54 2.8 Å |
| RT103A | RT52A | p66: K465R/P468K | | no crystals |
| RT104A | RT52A | p66: V466I/V467I/L469I | | RT104A/JLJ0135 |
| | | | | (NNRTI) a = 162 |
| | | | | b = 73.3 c = 108.9 |
| | | | | β = 100.3 C2 2.4 Å |
| | | | | RT104A/GMMP028 |
| | | | | (RNHI) |
| | | | | a = 163.22 b = 72.6 |
| | | | | c = 109.37 β = 108 |
| | | | | 2.9 Å |
| RT105A | RT52A | p66: K465R/V466I/V467E/P468K/ | | no crystals |
| | | L469I | | |
| RT106A | RT52A | p66: V466I/V467E/P468E | | RT106A/GMMP028 |
| | | | | (RNHI) |
| | | | | a = 163.22 b = 72.6 |
| | | | | c = 109.37 β = 108 |
| | | | | 2.9 Å |
| RT107A | RT52A | p66: K461R/K465R/V466I | | |
| RT109A | RT69A | D498N | | |
| RT110A | RT69A | E478Q | | No diffraction |
| RT113A | RT52A | p66: N348I | | |
| RT114A | RT55A | p66: N348I | | |

[symbol]

indicates data missing or illegible when filed

TABLE 10
Table 2. Crystallization Trial Kit developed for mutant RT's

A1. 50 mN bis-Tris propane pH 6.8 100 mM NH4SO4, 10% v/v glycerol,
8% w/V PEG8000
A2. 50 mM bis-Tris propane pH 6.8 100 mM NH4SO4, 10% v/v glycerol,
9% w/V PEG8000
A3. 50 mM bis-Tris propane pH 6.8 100 mM NH4SO4, 10% v/v glycerol,
10% w/V PEG8000
A4. 50 mM bis-Tris propane pH 6.8 100 mM NH4SO4, 10% v/v glycerol,
11% w/V PEG8000
A5. 50 mM bis-Tris propane pH 6.8 100 mM NH4SO4, 10% v/v glycerol,
12% w/V PEG8000
A6. 50 mM bis-Tris propane pH 6.4, 100 mM ammonium sulfate, 5%
(w/v) sucrose, 5% (v/v) glycerol, 10% (w/v) PEG 8000, 20 mM
MgCl2
B1. 33% saturated ammonium sulfate/100 mM sodium phosphate pH 6.8
B2. 34% saturated ammonium sulfate/100 mM sodium phosphate pH 6.8
B3. 35% saturated ammonium sulfate/100 mM sodium phosphate pH 6.8
B4. 1.4 M (NH4)SO4, 50 mM HEPES pH 7.2, 5 mM MgCl2, 300 mM
KCl
B5. 6% w/v PEG3400 at pH 5.0 with citrate/phosphate
B6. 7% w/v PEG3400 at pH 5.0 with citrate/phosphate
C1. 8% w/v PEG3400 at pH 5.0 with citrate/phosphate
C2. 9% w/v PEG3400 at pH 5.0 with citrate/phosphate
C3. 10% w/v PEG3400 at pH 5.0 with citrate/phosphate
C4. 50 mM Imidazole pH 6.4, 100 mM ammonium sulfate, 15 mM
MgSO4, 10 mM Spermine, 10% PEG 8000
C5. 50 mM Imidazole pH 6.4, 100 mM ammonium sulfate, 15 mM
MgSO4, 10 mM Spermine, 11% PEG 8000
C6. 50 mM Imidazole pH 6.4, 100 mM ammonium sulfate, 15 mM
MgSO4, 10 mM Spermine, 12% PEG 8000

The use of drug fragment cocktail screening (Bosch et al., 2006) is a potentially powerful technique for finding new inhibitors and sites for inhibition, but this approach was less feasible with the earlier, lower resolution RT crystals. Drug fragment cocktails are usually dissolved in DMSO prior to soaking of crystals in the DMSO plus crystallization solution. To determine if RT52A crystals could be used for fragment cocktail screening, crystals were soaked in 10-20% DMSO before and during cryoprotection. No loss in diffraction quality was found with 10% DMSO, and only a moderate decline in diffraction quality was found when 20% DMSO was used (2.0 Å versus 1.8 Å, data not shown). This result indicates that RT52A is a suitable construct for structure-based drug design through screening for binding of drug-like small chemical fragments and lead optimization at both existing and novel sites.

TABLE 11

| | | Length | |
|---|---|---|---|
| Name | Sequence | (nt) | Tm |
| 3CBAMH1 | GCGGATCCGGACCAAACACAGAATTTGCACTATCC | 35 | 83.1/61.5/67. |
| 3ccleavage | CGCCATGGCACATCACCACCACCATCACGCTCTTG | 77 | 94.5/72.9/ |
| | AAGTCCTCTTTCAGGGACCCATTAGCCCTATTGAGA | | 76.4 |
| | CTGTAC | | |
| 3CXho | CGGCTCGAGTTAGTTTCTCTACAAAATATTGTTTTTT | 48 | 81.8/60.2/ |
| | AAGTTGAGCTG | | 64.2 |
| ASLSSPfor | GAAAAGCAGGATATGTTACTAACAAAGGAGCATCA | 68 | 88.4/66.8/ |
| | CTGTCTAGCCCCCTAACTAACACAACAAATCAG | | 69.8 |
| ASLSSPrev | GCTCCTTTGTTAGTAACATATCCTGCTTTTC | 31 | 75.3/53.7/ |
| | | | 58.4 |
| C258Qfor | GACAGCTGGACTGTCAATGACATACAGAAGTTAGT | 51 | 85.9/64.3/68. |
| | GGGGAAATTGAATTGG | | |
| c258qrev | CTGTATGTCATTGACAGTCCAGCTGTC | 27 | 76.2/54.6/ |
| | | | 59.5 |
| catrem | GGATCCTTACGCCCCGCCCAGCTCTCTAGAGAGCT | 37 | 89.5/67.9/ |
| | CG | | 72.2 |
| d428revorf1 | CCAGCTCTCTAGAGAGCTCGGTGATGGTGATGGTG | 40 | 88./66.4/70.4 |
| | ATGGC | | |
| d428revorf2 | CCAGCTCTCTAGAGAGCTCGTTAGTGATGGTGATG | 43 | 87.5/65.9/ |
| | GTGATGGC | | 69.5 |
| d428revstorf1 | CCAGCTCTCTAGAGAGCTCGTTAGTGATGGTGATG | 43 | 87.5/65.9/ |
| | GTGATGGC | | 69.5 |
| D428stapsac | CGGGAGCTCTTACTGGTACCATAATTTCACTAAAGG | 39 | 83.1/61.5/ |
| | AGG | | 64.6 |
| D440stopsec | GTGGAGCTCTTAGAAGGTTTCTGCTCCTACTATGG | 35 | 81.9/60.3/ |
| | | | 63.7 |
| D447stopsac | GTGGAGCTCTTAGTTAGCTGCCCCATCTACATAGA | 37 | 82.9/61.3/ |
| | AG | | 64.8 |
| deltL92for | CTCAAGACTTCTGGGAAGTTCAAATACCACATCCC | 50 | 86./64.4/68.4 |
| | GCAGGGTTAAAAAAG | | |
| deltL92rev | GTATTTGAACTTCCCAGAAGTCTTGAG | 27 | 72.9/51.3/56. |
| deltw212for | GAGCTGAGACAACATCTGTTGAGGGGACTTACCAC | 49 | 86.6/65./69.3 |
| | ACCAGACAAAAAAC | | |
| deltw212rev | CCCCTCAACAGATGTTGTCTCAGCTC | 26 | 77.3/55.7/61. |
| e28afor | CAAAAGTTAAACAATGGCCATTGACAGCAGAAAAAA | 63 | 83.1/61.5/ |
| | TAAAAGCATTAGTAGAAATTTGTACAG | | 65.6 |
| e28arev | CTGCTGTCAATGGCCATTGTTTAACTTTTG | 30 | 75.4/53.8/ |
| | | | 60.1 |
| E297Afor | CACTAACAGAAGTAATACCACTAACAGCAGCAGCA | 60 | 89.1/67.5/ |
| | GCGCTAGAACTGGCAGAAAACAGAG | | 71.4 |
| E297Arev | CTGCTGTTAGTGGTATTACTTCTGTTAGTGC | 31 | 76.5/54.9/ |
| | | | 59.4 |
| E449Afor | GTAGATGGGGCAGCTAACAGGGCGACTAAATTAGG | 57 | 87.3/65.7/ |
| | AAAAGCAGGATATOTTACTAAC | | 69.1 |
| E449Arev | CGCCCTGTTAGCTGCCCCATCTAC | 24 | 78.8/57.2/ |
| | | | 63.3 |
| farforward | AGCCATACCGCGAAAGG | 17 | 65.6/44./54.2 |
| ForcesLIC | CGGGCTTTCTCCTTCCTCTCCCTTATGCGACTCC | 34 | 85.4/63.8/ |
| | | | 68.3 |
| forvectLIC3 | CGCTCCTCTTCGGGCCCGCCAGCACATGGACTCG | 34 | 90.3/68.7/ |
| | | | 74.7 |
| iso257for | CAGCTGGACTGTCAATGACATTTGTAAGTTAGTGG | 37 | 81.7/60.1/ |
| | GG | | 64.7 |
| iso257rev | ATGTCATTGACAGTCCAGCTG | 21 | 68.6/47./54.8 |
| K103Nfor | GAATACCACATCCCGCAGGGTTAAAAAAGAATAAAT | 57 | 86.1/64.5/ |
| | CAGTAACAGTACTGGATGTGG | | 67.9 |
| K172Afor | CAAAAATCTTAGAGCCTTTTGCAGCACAAAATCCAG | 59 | 83.6/62./65.7 |
| | ACATAGTTATCTATCAATACATG | | |
| K172Arev | GATTTTGTGCTGCAAAAGGCTCTAAGATTTTTGTCA | 39 | 79.8/58.2/64. |
| | TGC | | |
| K172Rfor | CATGACAAAAATCTTAGAGCCTTTTCGTAAACAAAA | 61 | 83.1/61.5/ |
| | TCCAGACATAGTTATCTATCAATAC | | 64.4 |
| K172RREV | ACGAAAAGGCTCTAAGATTTTTGTCATG | 28 | 71.7/50.1/ |
| | | | 56.8 |
| K201Afor | CTTAGAAATAGGGCAGCATAGAACAGCAATAGCGG | 58 | 89.5/67.9/ |
| | CGCTGAGACAACATCTGTTGAGG | | 71.9 |
| K201Arev | ATTGCTGTTGTATGCTGCCCTATTTCTAAG | 30 | 75.4/53.8/ |
| | | | 59.2 |
| K219Afor | GGGGAGTTACCACACCAGACGCAGCACATCAGAAA | 48 | 89.6/68./72.2 |
| | GAACCTCCATTCC | | |
| K219Arev | TGCGTCTGGTGTGGTAAGTCGCC | 23 | 76.8/55.2/ |
| | | | 62.6 |
| K223Afor | GTACAGCCTATAGTGCTGCCAGCAGCAGACAGCTG | 48 | 89.6/68./72.1 |
| | GACTGTCAATGAC | | |
| K223Arev | CTGCTGGCAGCAGTATAGGCTGTAC | 25 | 77.5/55.9/ |
| | | | 61.2 |
| K395Afor | GAAAGACTCCTAAATTTAAACTACCCATACAAGCGG | 58 | 87.5/65.9/ |
| | CAACATGGGAAACATGGTGGAC | | 69.7 |
| K395Arev | CGCTTGTATGGGTAGTTTAAATTTAGGAGTCTTTC | 35 | 77.4/55.8/ |
| | | | 59.7 |
| K4611Rfor | AAAAGCAGGATATGTTACTAACAGAGGAAGACAAA | 80 | 88.2/66.6/ |
| | AGGTTGTCACCCTAACTGACACAACAAATCAGAAAA | | 70.1 |
| | CTGAGTTAC | | |
| k461rrev | CCTCTGTTAGTAACATATCCTGCTTTTC | 28 | 73.4/51.8/ |
| | | | 55.8 |
| k512Qfor | GGAATCATTCAAGCACAACCAGATCAAAGTGAATCA | 60 | 84.6/63./66. |
| | GAGTTAGTCAATCAAATAATAGAG | | |
| k512qrev | TTTGATCTGGTTGTGCTTGAATGATTCC | 28 | 73.4/51.8/ |
| | | | 58.5 |
| K527Afor | GTCAATCAAATAATAGAGCAGTTAATAGCAAAGGCA | 59 | 88.1/66.5/ |
| | GCGGTCTATCTGGCATGGGTACC | | 69.9 |
| K527Arev | GCCTTTGCTATTAACTGCTCTATTATTTGATTGACTA | 39 | 77.7/56.1/60. |
| | AC | | |
| kozacatf | ACGCCACCATGGCTCACCATCATCAC | 26 | 78.9/57.3/ |
| | | | 65.1 |
| L100lk103Nfor | GAATACCACATCCCGCAGGGATAAAAAAGAATAAAT | 57 | 86.1/64.5/ |
| | CAGTAACAGTACTGGATGTGG | | 67.7 |
| ncomutfor | CTTCTTTCGCCCCCGTTTTCACGATGGGCAAATATTA | 44 | 85.4/63.8/ |
| | TACGCAAG | | 68.9 |
| ncomutrev2 | TGAAAACGGGGGCGAAGAAG | 20 | 70.3/48.7/ |
| | | | 57.6 |
| orf1-2rbsfor | TAGAAATAATTTTGTTTAACTTTAAGAAGGAGATATA | 38 | 72.3/50.7/54. |
| | C | | |
| orf13primfor | CGAGCTCTCTAGAGAGCTGG | 20 | 72.3/50.8/ |
| | | | 55.5 |
| orf13primrev | CGCGGGATCGAGATCAACGC | 20 | 74.4/52.8/ |
| | | | 60.7 |
| orf1rev5prim | GTTTAACTTTAAGAAGGAGATATACCATGGTT | 32 | 73.1/51.5/ |
| | | | 55.7 |
| orf1rev5prim | AACCATGGTATATCTCCTTCTTAAAGTTAAAC | 32 | 73.1/51.5/ |
| | | | 55.7 |
| orf1rev5prim | AACCATGGTATATCTCCTTCTTAAAGTTAAAC | 32 | 73.1/51.5/ |
| | | | 55.7 |
| orf2 p66rev | GAAACTTTACCAGACTCGAGTTAAAGTATTTTCCTG | 44 | 81.7/60.1/ |
| | ATTCCAGC | | 63.9 |
| orf2hisrev | GAAACTTTACCAGACTCGAGTTAGTGATGGTGGTG | 41 | 83.9/62.3/ |
| | GTGATG | | 66.1 |
| orf2nderev | CATATGTATATCTCCTTCTTAAAGTTAAAC | 30 | 69.7/48.1/ |
| | | | 51.1 |
| orf2p66IE | AGAAGGAGATATACATATGCATATGGTTATTGAGAC | 55 | 81.5/59.9/ |
| | TGTACCAGTAAAATTAAAG | | 62.8 |
| orf2p66PIE | AGAAGGAGATATACATATGCATATGGTTCCTATTGA | 58 | 83./61.4/64.1 |
| | GACTGTACCAGTAAAATTAAAG | | |
| orf2p66PISP | AGAAGGAGATATACATATGCATATGGTTCCCATTAG | 48 | 83.4/61.8/65. |
| | CCCTATTGAGAC | | |
| Orf2vrevLIC | CACGCGGGCGGGCGTTGGATCCCCCCGGGTCC | 32 | 93.6/72./78.7 |
| Orf2vrevLIC2 | CACGGGGGCGGCCGTGGATCCTTACGCCCCGC | 32 | 92.4/70.8/ |
| | | | 77.6 |
| orfcasforLIC | CGGCCGCCCGCGTGTGTTGATCTCGATGCCGCG | 33 | 90.6/69./75.7 |
| P51licrevx | GAGGAGAAGCCCGGTCTCGAGTTAGTGATGGTGG | 42 | 88.8/67.2/71. |
| | TGGTGATG | | |
| p5lorf1stop | GCGAGCTCTTAGTGATGGTGGTGGTGATG | 29 | 80.8/59.2/ |
| | | | 64.4 |
| p66D555xho | CGGCTCGAGTTATCCAGCACTGACTAATTTATCTAC | 39 | 81.8/60.2/ |
| | TTG | | 63.9 |
| P66licrevx | GAGGAGAAGCCCGGTCTCGAGTTATAGTATTTTCC | 47 | 86.8/65.2/ |
| | TGATTCCAGCAC | | 68.8 |
| p66orf1ie | CTCCATGGTTATTGAGACTGTACCAGTAAAATTAAA | 37 | 77.6/56./59.3 |
| | G | | |
| p66orf1IE | CTTTAAGAAGGAGATATACCATGGTTATTGAGACTG | 54 | 82.5/60.9/ |
| | GTACCAGTAAAATTAAAG | | 63.2 |
| p66orf1pie | CTCCATGGTTCCTATTGAGACTGTACCAGTAAAATT | 40 | 79.8/58.2/ |
| | AAAG | | 61.4 |
| P66orf1PIE | CTTTAAGAAGGAGATATACCATGGTTCCTATTGAGA | 46 | 83.6/62./64.3 |
| | CTGGTACCAG | | |
| P66orf1PISP | CTTTAAGAAGGAGATATACCATGGTTCCCATTAGCC | 46 | 83.6/62./64.6 |
| | CTATTGAGAC | | |
| p66orf1pispie | CTCCATGGTTCCCATTAGCCCTATTGAGAC | 30 | 79.5/57.9/ |
| | | | 61.9 |
| p66orf1stop | CAGAGCTCGTTACAGTATTTTCCTGATTCCAGCACT | 39 | 83.1/61.5/ |
| | GAC | | 65.5 |
| p66orf2licr | TAGGGCTAATGGGAACCATATG | 22 | 69.3/47.7/ |
| | | | 53.4 |
| p66orf2rev | GTTTCTTTACCAGACTCGAGTTATAGTATTTTCCTGA | 48 | 83.4/61.8/ |
| | TTCCAGCACTG | | 65.1 |
| p66revorf1 | CAGTGCTGGAATCAGGAAAATACTACGAGCTCTCT | 45 | 86.2/64.6/ |
| | AGAGAGCTGG | | 67.9 |
| p66revorf1 | CCAGCTCTCTAGAGAGCTCGTAGTATTTTCCTGATT | 45 | 86.2/64.6/ |
| | CCAGCACTG | | 67.9 |
| p66revstorf1 | CCAGCTCTCTAGAGAGCTCGTTATAGTATTTTCCTG | 48 | 85.9/64.3/ |
| | ATTCCAGCACTG | | 67.3 |
| remCAT | GCCCCCGGGAGCCAGCTCTCTAGAGAGCTC | 30 | 87.7/66.1/ |
| | | | 70.5 |
| remtermrev | GCCGGATCCTTACGCCCCGCCCTGCC | 26 | 86.7/65.1/ |
| | | | 72.3 |
| revcasLIC3 | GGCCCGAAGAGGAGCGCCGGTTTCTTTACCAGACT | 39 | 89.2/67.6/ |
| | CGAG | | 72.4 |
| RevvectLIC | GAGGAGAAAGCCCTGCGTCGAGATCCCGGAC | 31 | 86./64.4/69.4 |
| revvectLIC2 | GAGGAGAAAGCCCGGGTATGGCATGATAGCGCC | 33 | 85.6/64./69.4 |
| sacforterm | CCGGAGCTCTCTAGAGAGCTGGCTC | 25 | 80.7/59.1/ |
| | | | 63.4 |
| SeqCas2Revb | CTCAGGTACACGACCGCAAG | 20 | 72.3/50.8/ |
| | | | 57.5 |
| SeqCas2Revc | GAGTCCATGTGCTGGCGTTC | 20 | 72.3/50.8/ |
| | | | 58.4 |
| seqrt454rev | TCCTTTGTTAGTAACATATCCTGC | 24 | 68.5/46.9/ |
| | | | 52.7 |
| termmutfor | CGGGATCCAGCAAAAAACCCCTCAAGACCCGTTTA | 39 | 85.9/64.3/ |
| | GAGG | | 69.2 |
| y181cfor | CCTTTTAAAAAACAAAATCCAGACATAGTTATCTGT | 65 | 83.4/61.8/ |
| | CAATACATGGATGATTTGTATGTAGGATC | | 64.8 |
| y181crev | AGATAACTATGTCTGGATTTTGTTTTTTAAAAGG | 34 | 72.3/50.7/ |
| | | | 55.7 |
| y188Lfor | GACATAGTTATCTATCAATACATGGATGATTTGCTT | 61 | 85.2/63.6/ |
| | GTAGGATCTGACTTAGAAATAGGGC | | 65.8 |
| y188Lrev | CAAATCATCCATGTATTGATAGATAACTATGTC | 33 | 73.3/51.7/ |
| | | | 54.8 |
| catorflic1 | GACGACGACAAGATCCATGGACTACIGAGTGAGAG | 38 | 85.1/63.5/ |
| | CTC | | 67.1 |
| catrevlicadadapt | CGCGGGCGGCCGGGATCCCCCCGGGTCCTCCTTT | 35 | 95./73.4/79.2 |
| | C | | |
| catrevlicsing | GAGGAGAAGCCCGGTGGATCCCCCCGGGTCCTCC | 38 | 91.6/70./74.4 |
| | TTTC | | |
| seqforcas2 | TTGATCTCGATCCCGCG | 17 | 65.6/44./53.7 |
| seqrevcas2 | GCTAGTTATTGCTCAGCGGTG | 21 | 70.7/49.1/56. |
| seqforcas1 | GCTCTCCCTTATGCGACTCC | 20 | 72.3/50.8/ |
| | | | 56.7 |
| seqrevcas1 | CGGGATCGAGATCAACGCG | 19 | 71.8/50.2/58. |
| Y181172Afor | CCTTTTGCAGCACAAAATCCAGACATAGTTATCTGT | 65 | 65.9/64.3/67.7 |
| | CAATACATGGATGATTTGTATGTAGGATC | | |
| Y181172Arev | AGATAACTATOTCTGGATTTTGTGCTGCAAAAGG | 34 | 77.2/55.6/ |
| | | | 61.2 |
| p66plus560 | CGGCTCGAGTTATAGTATTTTCCTGATTCCAGCACT | 54 | 84.6/63./66. |
| | GACTAATTTATCTACTTG | | |
| no3ccleavage | CGCCATGGCACATCACCACCACCATCACGCTCTTC | 59 | 92.2/70.6/ |
| | CCATTAGCCCTATTGAGACTGTAC | | 74.6 |
| p51ietv | CGCCATGGGACATCACCACCACCATCACGCTCTTG | 79 | 91.8/70.2/74. |
| | AAGTCCTCTTTCAGGGAATTGAGACTGTACCAGTAA | | |
| | AATTAAAG | | |
| p66d552 | CGGCTCGAGTTAGACTAATTTATCTACTTGTTCATTT | 42 | 81.8/60.2/ |
| | CCTCC | | 63.5 |
| R307Arev | CTTTTAGAATCTCTGCGTTTTCTGCCAGTTCTAGCT | 40 | 83.1/61.5/ |
| | CTGC | | 65.8 |
| R307A | GCAGAAAACGCAGAGATTCTAAAAGAACCAGTACA | 40 | 81.8/60.2/ |
| | TGGAG | | 64.6 |
| E248Arev | CAGCTGTCTTTTGCTGGGAGCACTATAGGCTGTACT | 37 | 84.2/62.6/ |
| | G | | 67.4 |
| E248A | CTGCCAGCAAAAGACAGCTGGACTGTCAATGACAT | 37 | 82.9/61.3/ |
| | AC | | 66.5 |
| I125N136E138Are | CCCTGGTGTCGCATTGGCTGCACTAGGTATGGTAA | 49 | 87.4/65.8/70. |
| v | ATGCAGTATACTTC | | |
| I135N136E138A | GCAGCCAATGCGACACCAGGGATTAGATATCAGTA | 47 | 86.8/65.2/ |
| | CAATGTGCTTCC | | 69.8 |

The Tm field contains three melting temperature values calculated in the program AmplifX (http://ifrjr.nord.univ-mrs.fr/AmplifX). The melting temperatures of the oligonucleotide from the template are calculated three different ways: the standard simple and approximate method: TM = 81.5 + 0.41*GC-675/N, the second takes the salt concentration in a PCR
reaction into account TM = 81.5 + 16.6 × log10([Na+] + [K+]) + 0.41 × (% GC)-675/N (with default values: [Na+] + [K+] = 0.05 (50 mM)), the third is the most precise called the bases stacking method TM = ΣΔH/(ΣΔS + 0.368 × Nxln[Na+] + R × ln[Primer]/4) with R = 1.987 and the different ΔH and ΔS taken in Santalucia (1998). Santalucia J PNAS 95 pp1460-1465
(1998)

### Validation of RT52A and Derivatives

Comparison of the RT52A/TMC278 structure with 1B1 RT/NNRTI structures showed that the fold for RT, distribution of secondary structure elements, and mode of NNRTI binding (Das et al 2008) are very similar, suggesting no significant impact of crystal engineering mutation on structure and functions of RT. Proteins RT35A (RT52A without the K172A/K173A mutation), RT51A (RT52A+L100I/K103N), RT52A, and RT55A (RT52A+K103N/Y181C) were tested for DNA-dependent DNA polymerase processivity and RNase H activity. FIG. 18A shows that RT52A has similar processivity as wild-type HIV-1 RT (RT p66 co-expressed with HIV-1 protease), with RT51A having a diminished processivity and RT55A an increase. Each of the mutants has similar RNase H activities (FIG. 18B).

Engineering of High-Resolution apo-RT Crystals

While RT52A successfully produced crystals of RT/NNRTI complex diffracting to high resolution, the unliganded RT52A crystals diffracted only to ˜3 Å resolution (Table 9). The apo-form of 1B1 RT (Hsiou et al. 1996) crystallizes with different unit cell parameters compared to those of RT/NNRTI complexes. The difference in the unit cell parameters between RT and RT/NNRTI crystals is a consequence of packing of two structurally distinct (thumb up vs. down) conformations of RT. This may explain why RT52A, which is optimized to produce RT/NNRTI crystals diffracting to high resolution, fails to do so if crystallized without an NNRTI. A different set of mutations may therefore be necessary to obtain a high-resolution apo-RT crystal form.

Subsequent rounds of mutagenesis were focused on obtaining high-resolution crystals of apo enzyme and in complexes with RNase H inhibitor (RNHI) bound or DNA bound RT. One of the successful mutants for apo and RNHI-bound crystals is RT69A which contains an adventitious mutation F160S; this construct yields crystals that diffract X-rays to 1.8 Å resolution. Another mutant with improved crystals for RNHIs is RT97A, which contains the mutations P468T/N471D in addition to the RT52A mutations. RT97A produces crystals that diffract X-rays to 2.1 Å resolution. Thermal stability assays of various mutants using circular dichroism did not show any significant in-solution stability changes that would lead to the observed improvement in diffraction quality (FIG. 19).

### Discussion

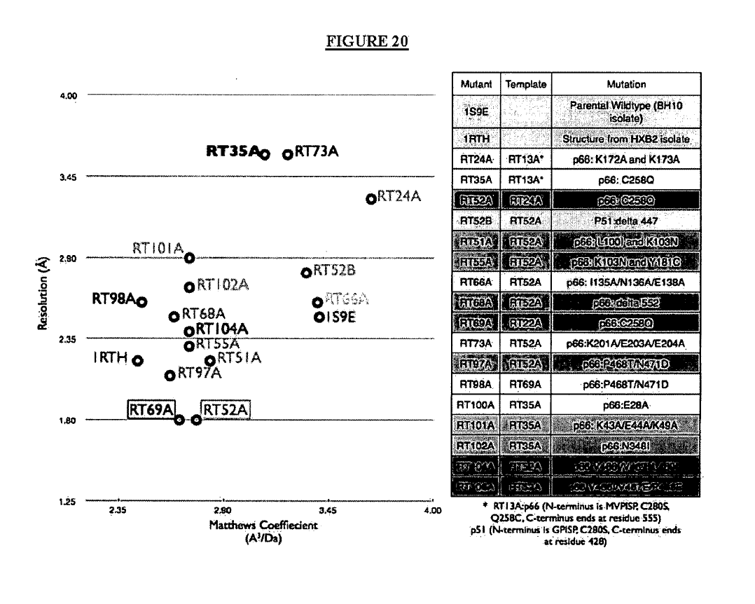

In general, there is no rationale for crystallization of proteins and improvement of diffraction quality of crystals, although it is highly desirable and remains challenging. Our successful approach of using protein engineering to improve the resolution from ˜6 Å to 1.8 Å of a very important HIV-1 drug complex has implications in designing anti-AIDS drugs and also provides a rare example of the use of rational approaches in enhancing the diffraction quality of macromolecular crystals. Reverting each of the mutations of RT52A either caused a loss in crystallization or diminished diffraction quality. Further mutagenesis showed that the unit cell of RT52A/NNRTI complexes is primarily defined by the termini mutations, whereas, the other mutations have additive effects in increasing the X-ray diffraction resolution (FIG. 20 and Table 9). We propose that the unit cell of RT52A and the very similar unit cell of RT69A (except ˜4° difference in b) are determined primarily by the termini of the construct, and the residue substitutions cause a stabilizing effect on the crystallized conformation of RT. The stabilization of a crystallized conformation within the confines of tighter crystal packing is thereby responsible for the improved diffraction (FIG. 20).

A flexible enzyme like RT that exhibits hinge movements may assume different conformations in solution and becomes relatively homogeneous when complexed with a ligand that may favor a single conformation or a subset of conformations. Different types of ligands enrich specific subsets of RT conformations and therefore favor formation of different crystal forms. In the process of protein engineering for crystallization, it became clear that the engineering protocol must be applied and optimized separately for different conformations of RT induced by binding of distinctive types of ligand and substrates. The ligand specificity of crystallization has led to protein engineering being applied to other types of RT complexes that have been resistant to structural studies in the past. RT69A is the first of the successful constructs tested with non-NNRTI ligand specificity in mind. RT69A contains the mutation F160S that is located adjacent to the binding cleft for nucleic acid near the polymerase site. Therefore, RT69A may not be the optimal construct for studies near the polymerase active site; however, it is a suitable construct for structural studies of RT in complexes with RNHIs. RT97A does not contain the F160S loss-of-function mutation but with X-ray diffraction resolution of 2.1 Å can be readily used for studies of RNHIs. Further work with current constructs as well as further mutagenesis should provide high-resolution structures of RT in different functional states, especially those with bound nucleic acid template-primers.

The approach of the present invention was successful in finding a RT mutant that gave diffraction quality crystals in the presence of TMC278. The superior crystallizability and diffraction quality obtained by crystal engineering demonstrates the usefulness of a systematic iterative mutagenesis approach for improving crystallization of critical drug targets and functionally important macromolecules. This success has led to the feasibility of doing high-throughput crystallization of RT in complex with NNRTIs. It is now possible to produce high-resolution diffraction within days of starting crystal trials with a new inhibitor. This opens up new possibilities of structure-based drug design through drug candidate co-crystallization studies as well as fragment screening (Hartshorn et al., 2005).

### Example 4

### High Resolution Structures of HIV-1 RT/TMC278 Complexes: Strategic Flexibility Explains Potency Against Resistance Mutations

1. Expression, Purification, and Crystallization

The IB1 RT used in earlier crystallographic studies of RT/NNRTI complexes produced RT/TMC278 crystals that diffracted to only 6 Å resolution. To overcome this obstacle and obtain suitable diffraction data for structural studies, a systematic crystal engineering approach that improved resolution was employed. The RT used in the current crystallographic analyses reported here were developed using this strategy. The RT/TMC278 complexes were crystallized from an engineered RT at 20 mg/ml in 10 mM Tris pH 8.0, 75 mM NaCl containing TMC278 with a 5:1 molar ratio of TMC278 to RT. Crystals were obtained in hanging drop vapor diffusion setups at 4° C. The well solution contained 12% PEG 8000, 100 mM ammonium sulfate, 10 mM MgCl₂, 15 mM spermine, and 50 mM imidazole buffer at pH 6.8. The crystals grew to appropriate size for diffraction within one week. Crystals of the RT/TMC278 complexes were dipped for 10 seconds in their respective mother liquors containing 25% ethylene glycol for cryoprotection. The cryoprotected crystals were flash-cooled in liquid N₂ and transported to synchrotron sources.

2. Structure Solution

Diffraction data were collected from one crystal of each type of RT/NNRTI complex at the Cornell High Energy Synchrotron Source (CHESS) F1 beam line. The data were processed using HKL2000. The engineered RT/TMC278 complexes crystallized in a new crystal form. The previously reported structure of the RT/R147681 complex was used as a starting model for obtaining molecular replacement solutions for the structure of the wild-type RT/TMC278 complex. The 1.8 Å resolution structure of the wild-type RT/TMC278 complex was used as the starting model for obtaining the structures of I100L/K103N mutant and K103N/Y181C mutant RTs in complexes with TMC278. The final models for the three structures were obtained after cycles of model building in COOT and restrained refinement using REFMAC and CNS 1.1. The high resolution structure of the RT/TMC278 complex revealed no metal binding at the polymerase active site. Also, no metal ion with clear coordination geometry could be located at the RNase H active site. An electron density peak that is nearly the positional equivalent of a metal cation at the RNase H active site, however, was assigned as a water as its lacks the proper metal coordination. Coordinates and structure factors for the structures of wild-type RT/TMC278, K103N/Y181C/TMC278, and L100I/K103N/TMC278 complexes are available from the Protein Data Bank with PDB IDs 2ZD1, 3BGR, and ZZZ, respectively.

### HIV-1 RT/TMC278 Complex

The present invention describes the structure of wild-type HIV-1 RT complexed with TMC278 at 1.8 Å resolution, using a new RT crystal form engineered by systematic RT mutagenesis. This high resolution structure reveals that the cyanovinyl group of TMC278 is positioned in a hydrophobic tunnel connecting the NNRTI-binding pocket to the nucleic acid-binding cleft. The crystal structures of TMC278 in complexes with the double mutant K103N/Y181C (2.1 Å) and L100I/K103N HIV-1 RTs (2.9 Å), demonstrated that TMC278 adapts to bind mutant RTs. In the K103N/Y181C RT/TMC278 structure, loss of the aromatic ring interaction caused by the Y181C mutation is counter balanced by new interactions between the cyanovinyl group of TMC278 and the aromatic side chain of Y183, which is facilitated by an ˜1.5 Å shift of the conserved Y₁₈₃MDD motif. In the L100I/K103N RT/TMC278 structure, the binding mode of TMC278 is significantly altered so that the drug conforms to changes in the binding pocket primarily caused by the L100I mutation. The flexible binding pocket acts as a molecular “shrink wrap” that makes a shape complementary to the optimized TMC278 in wild-type and drug-resistant forms of HIV-1 RT. The crystal structures provide a better understanding of how the flexibility of an inhibitor can compensate for drug resistance mutations.

A systematic protein engineering approach according to the present invention was used to obtain a mutant form of RT that yielded better diffracting crystals of the RT/TMC278 complex. Successful protein engineering included: (i) truncating the termini of the protein; (ii) removing surface lysine and glutamic acid patches; and (iii) altering amino acid residues to make new lattice contacts and/or remove some of the lattice contacts seen in earlier crystal forms. This mutated RT produced crystals of the HIV-1 RT/TMC278 complex in a new crystal form that is distinct from the reported crystals of RT/NNRTI complexes. One of the new crystal forms of the engineered RT/NNRTI complexes diffracted X-rays to 1.8 Å, significantly better than any of the reported structures of HIV-1 RT. The L100I/K103N and K103N/Y181C double mutant (in the p66 subunit only) RTs were designed based on the above construct, and their structures in complexes with TMC278 were determined at 2.9 and 2.1 Å resolution, respectively.

### Results

### Engineering RT for High Resolution Diffraction

Numerous earlier attempts to obtain a crystal structure of the HIV-1 RT/TMC278 complex failed and the best crystals diffracted X-rays to only 6 Å resolution. In contrast, the engineered RT/TMC278 complex crystallized in a new form and the crystals diffracted X-rays to 1.8 Å resolution (Table 2). The structure of wild-type HIV-1 RT/TMC278 was determined by molecular replacement using the structure of RT/R147681 (PDB ID 1S6Q) as the starting model and refined to 1.8 Å resolution to R-work and R-free of 0.221 and 0.248, respectively. This high resolution structure of HIV-1 RT has excellent stereochemistry (>91% of amino acid residues are in the most favored regions of the Ramachandran plot with no outliers; Procheck G-factor=0.25) and a reliable solvent model. The form of recombinant RT (1B1) used in previous structural studies of RT/NNRTI complexes crystallized with the symmetry of space group C2 with unit cell volume ˜1.6×10⁶ Å³, one molecule/asymmetric unit, approximate solvent content of 64%, and a Matthews coefficient of 3.4. The p66 fingers and thumb subdomains are flexible and not involved in any significant crystal contacts. Earlier structural studies have shown that NNRTI binding is accompanied by repositioning of the thumb and fingers subdomains, resulting in a conformation of RT with a wide cleft between these mobile subdomains. Comparing the crystal structures of a number of RT/NNRTI complexes revealed that individual NNRTIs have both short-range and long-range effects on the conformation of RT, and affect the precise positioning of the p66 thumb and fingers subdomains. Several DAPY inhibitors, by virtue of their structural flexibility and compactness, are predicted to have the ability to bind RT in more than one conformation. These different binding modes for a single NNRTI may also lead to differences in the positions of the fingers and thumb. In the context of a crystal lattice, this heterogeneity in the arrangement of RT molecules would reduce the resolution of X-ray diffraction from the crystal.

An engineered RT variant (RT52A) crystallized in a new form with the symmetry of C2 space group and a unit cell volume of ˜1.3×10⁶ Å³. The unit cell volume and the solvent content are reduced by ˜18% and 28%, respectively, compared to the C2 crystal form obtained with the parental RT. The lower solvent content of ˜56% with a Matthews coefficient of 2.8, compared to 64% solvent of the old C2 unit cell with a Matthews coefficient of 3.4, reflects significantly tighter packing of the RT molecules in the crystal lattice. The new crystal form involves new protein contacts; of the new contacts, a set of back-to-back interactions between the p66 thumb and p66 fingers of symmetry-related RT molecules may be critical in stabilizing the positions of p66 thumb and fingers subdomains in the new crystal lattice (Supplementary FIG. S1). The tighter packing of the engineered RT molecules and the specific intermolecular interactions seen with this form of RT may have contributed to the higher order and high resolution (1.8 Å) diffraction. A total of 113,072 unique reflections were used to refine the structure of one RT molecule, which is about 2.3 times the number of observations (49,347 reflections) used in refining the published highest resolution (2.2 Å) structure (PDB ID 1VRT) of an HIV-1 RT/NNRTI complex. The substantial increase in experimental measurements leads to higher accuracy and greater overall reliability of the current structure.

Individual subdomains of the engineered wild-type (RT52A) RT/TCM278 structure and 1B1 RT/TMC120 structure are highly similar. The overall Ca atom superposition of the structures had an rmsd of 1.6 Å, primarily due to small differences in the relative positioning of the subdomains. The Ca superposition of the binding pocket regions of both structures (residues 98-110, 178-190, and 226-240 of the p66 subunit) showed an rmsd of 0.85 Å. The overall similarity in the binding of the two DAPY compounds also suggests only subtle or modest effects of the crystal-engineered mutations on the inhibitor binding. The engineered RT52A also exhibited both DNA polymerization and RNase H activities similar to 1B1 RT.

### 1.8 Å Resolution Structure of the Wild-Type HIV-1 RT/TMC278 Complex

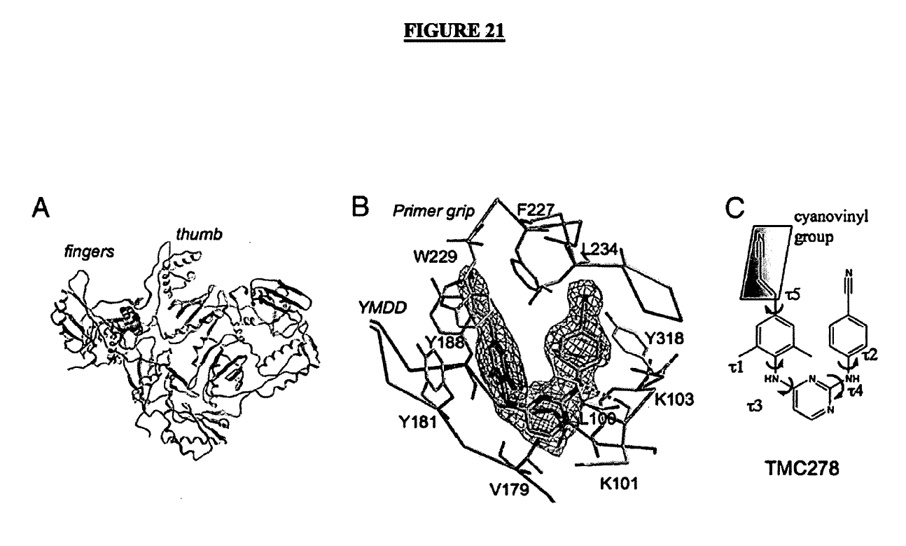

Overall, the structure of the p66/p51 RT heterodimer (FIG. 21A) in the HIV-1 RT/TMC278 complex resembles the open-cleft conformation seen in the previous structures of RT/NNRTI complexes. The electron density maps (FIG. 21B) unambiguously defined the position and conformation of TMC278 in the structure of HIV-1 RT/TMC278 complex. TMC278 has a conformation that is similar to the horseshoe conformation seen with other DAPY inhibitors, with the three aromatic rings connected by two linking amino groups, and a cyanovinyl (acrylonitrile) substituent that is unique to TMC278 (FIG. 21C). The torsion angles of the rotatable bonds (τ1-τ4) of TMC278 have values similar to those of the prototype DAPY analog TMC120 (R147681/dapivirine) bound to RT, although the two structures were determined in two different crystal forms using two different RT constructs.

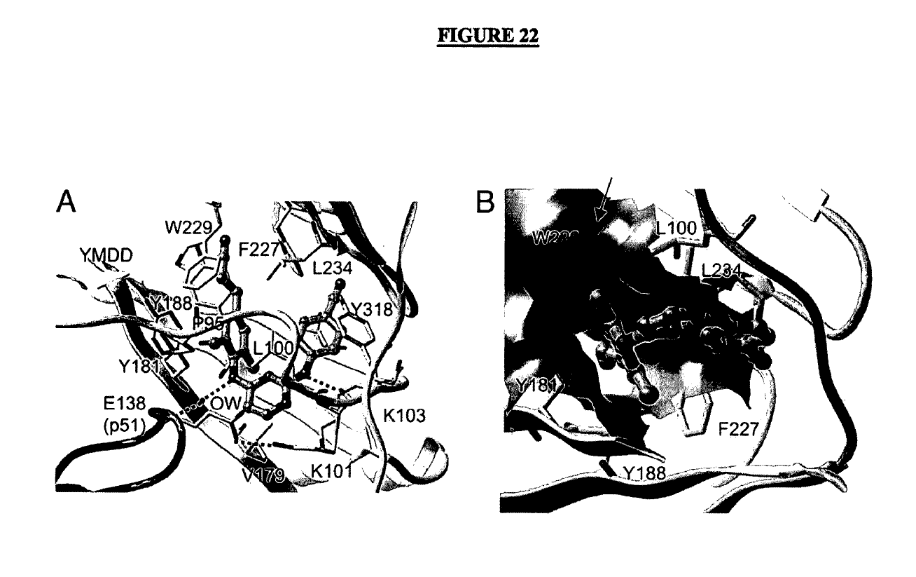

TMC278 makes important contacts with a number of key amino acids in the NNRTI binding pocket (FIG. 22). The hydrogen bond between a linker nitrogen atom of TMC278 and the main-chain carbonyl oxygen of K101 is conserved in the binding of many NNRTIs. The second linker nitrogen is involved in a water-mediated hydrogen bond network with the main-chain carbonyl group of E138 of the p51 subunit (FIG. 22A). The dimethylphenyl ring and its attached 4-cyanovinyl group interact with the hydrophobic core of the binding pocket. The cyanovinyl group is positioned to fit into a hydrophobic tunnel formed by the side chains of amino acid residues Y188, F227, W229, and L234; this tunnel opens toward the nucleic acid-binding cleft (FIG. 22B). A similar tunnel was seen in the binding of a cyanovinyl-containing iodo-pyridinone (IOPY) NNRTI (PDB ID: 2B5J). In the free TMC278 molecule, the cyanovinyl group is expected to be coplanar with the dimethylphenyl ring. However, in the RT-bound conformation, the plane of the cyanovinyl group is inclined 45° to the plane of the dimethylphenyl ring. The extensive interactions of the cyanovinyl group with the hydrophobic tunnel may explain why TMC278 is the most potent of the DAPY analogs.

The high resolution structure provides a reliable solvent model. The amino acid residues K101 and K103 are solvent exposed (FIG. 22A) and, if mutated, each can confer NNRTI resistance. In the RT/TMC278 structure, the Nζ atom of K103 interacts with two water molecules whereas the corresponding Nζ of K101 interacts with four oxygen atoms: the carbonyl oxygen of G99, both carboxyl oxygen atoms of E138 (of the p51 subunit), and a water molecule. The location of the K101-N□ atom in the TMC278 complex is similar to that in the recently published structure of the HIV-1 RT/GW420867X complex; however, the identification of the interaction between K101-Nζ and four surrounding oxygen atoms including one from a solvent water molecule defines a novel polar environment for K101. The different environments for and interactions of K101 and K103 may help account for the differences in resistance seen when these two lysines are mutated, even though both of their side chains point toward a common putative entrance to the NNRTI-binding pocket.

### Structure of the K103N/Y181C Double Mutant RT/TMC278 Complex

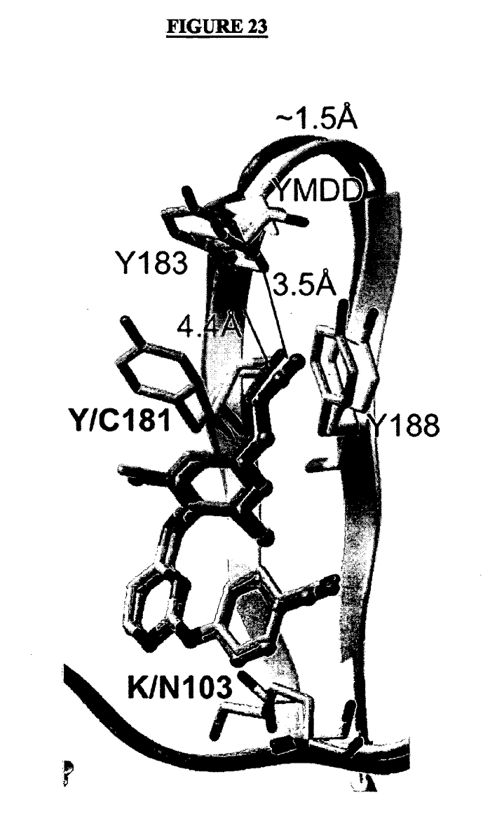

K103N and Y181C are the two resistance mutations most frequently observed in patients treated with NNRTIs, and viruses carrying these mutations show high levels of resistance to existing NNRTIs. However, TMC278 inhibits K103N, Y181C, and K103N/Y181C RT mutants at an EC₅₀<1 nM. The crystal structure of the K103N/Y181C mutant RT/TMC278 complex was determined at 2.1 Å resolution with R-work and R-free of 0.228 and 0.269, respectively. Superposition of this structure onto the wild-type RT/TMC278 structure revealed no major conformational changes for the bound TMC278 (FIG. 23). The number of distances <4.5 Å between pairs of atoms, one from RT and the other from TMC278, was used as an indicator of the extent of the hydrophobic interactions between RT and TMC278. In the K103N/Y181C mutant RT/TMC278 complex, the number of such distances is 51, which is almost same number of distances in the wild-type RT/TMC278 complex. A slight tilt (5°) of τ3 results in displacement of the dimethylphenyl-4-cyanovinyl group away from the mutated Y181C side chain. The interaction between the dimethylphenyl ring of TMC278 and the aromatic side chain of Y181 is lost, and a void is created by the mutation. In the structure of the mutant RT, the amino acid residue Y183, which is part of the conserved Y₁₈₃MDD motif at the polymerase active site, is shifted by ˜1.5 Å toward the NNRTI-binding pocket, permitting it to participate in the binding of TMC278 by binding the cyanovinyl group (FIG. 23).

The ability of the cyanovinyl group of TMC278 to recruit Y183 helps to compensate for the loss of interactions due to Y181C mutation; the involvement of Y183 in this compensatory interaction is particularly fortuitous and significant because Y183 is completely conserved in all HIV-1 sequences. This mode of compensatory interaction is different from that observed for another NNRTI, HBY 097, which developed a hydrogen bond with the thiol group of the mutated C181 side chain; in the K103N/Y181C mutant RT structure, the thiol group of C181 has a hydrogen bond with a water molecule at the equivalent position of the Oh atom of Y181 in the wild-type RT/TMC278 structure. Subtle conformational changes (as reflected by the torsion angles r of TMC278 in the K103N/Y181C mutant RT/TMC278 complex enhance the interactions of the cyanovinyl group with the modified hydrophobic tunnel (FIG. 22B), supplementing the contributions of the novel interactions with Y183. The extent of the interaction between the other mutated amino acid, N103, and TMC278 is comparable to the interaction between K103 of wild-type RT and TMC278: the number of distances <4.5 Å between the atoms of TMC278 and the amino acid K/N103 are 16 and 17, respectively, in the wild-type and the mutant structures.

### Structure of the L100I/K103N Mutant RT/TMC278 Complex

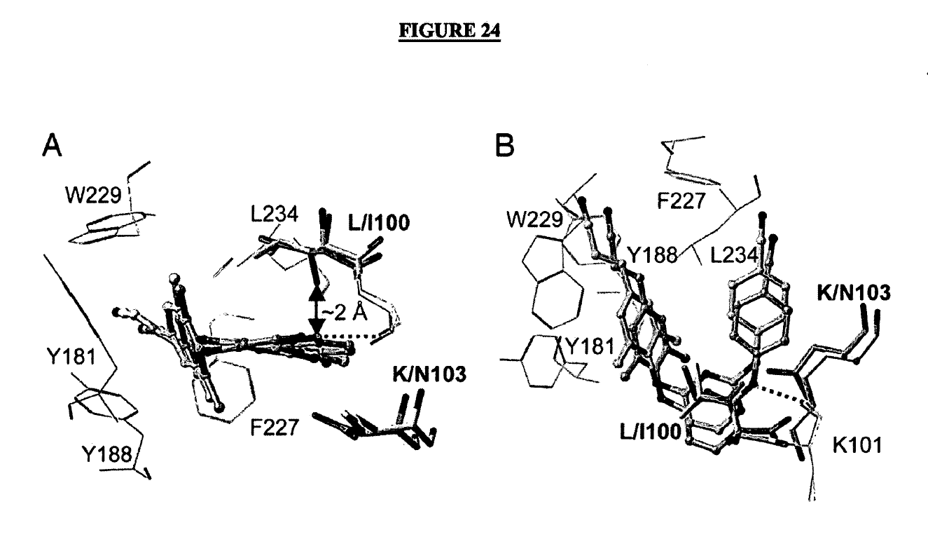

Among the known NNRTI-resistance mutations the L100I/K103N double mutation has the greatest effect on the potency of TMC278. However, TMC278 still inhibits the double mutant at ˜8 nM EC₅₀ (Table 1). The crystal structure of L100I/K103N mutant RT/TMC278 complex was determined at 2.9 Å resolution. The refined structure has R and R-free of 0.240 and 0.299, respectively. In the wild-type RT/TMC278 structure, L100 is near the center of the pocket and primarily interacts with the central pyrimidine ring of TMC278; K103 is located on the other side of the pyrimidine ring. Comparison of the structures of the L100I/K103N mutant RT/TMC278 and wild-type RT/TMC278 complexes (FIG. 24) shows that B-branching of 1100 in the L100I mutant would lead to steric conflict with the inhibitor if TMC278 were to bind in a conformation similar to that seen in the wild-type RT/TMC278 complex; in the mutant structure the C^{γ}2 atom of 1100 would be only ˜2 Å away from the position of the central pyrimidine ring of TMC278 when it is bound to wild-type RT. However, when TMC278 binds to the L1001/K103N mutant RT the drug undergoes significant conformational (wiggling) and positional (jiggling) rearrangements compared to the position is which it binds to wild-type RT (FIG. 24). To avoid steric conflict with the L100I mutation TMC278 shifts away from 1100 and towards N103 (FIG. 24A); the position of the entire inhibitor molecule is displaced by ˜1.5 Å in the pocket. The number of distances <4.5 Å between TMC278 and I100 is 13 in the complex with the L1001/K103N mutant RT, which is considerably less than the 28 and 30 distances <4.5 Å in the complexes with wild-type RT and K103N/Y181C mutant; however, in compensation, the number of protein-ligand distances <4.5 Å for residue 103 increases from 16 and 17 in the wild-type and K103N/Y181C mutant structures, respectively, to 27 in the L100I/K103N mutant RT/TMC278 structure.

In the L100I/K103N complex, the rotatable torsion angles τ1-τ5 of TMC278 are changed by 18, 18, 5, 22, and 45°, respectively, with respect to the wild-type RT/TMC278 complex. Unlike its configuration in the wild-type RT/TMC278 and Y181C/K103N mutant RT/TMC278 structures, the cyanovinyl group is almost co-planar with the dimethylphenyl ring in the L100I/K103N mutant RT/TMC278 structure. In the L100I/K103N complex structure the amino acid residues in the NNRTI-binding pocket are rearranged to optimize the inhibitor-protein interactions. This contrasts with an earlier proposal that the basis of the effects of the L100I mutation was a loss of interactions with Y181 and Y188. However, analysis of all of the structural results shows that L100I introduces a significant distortion in the NNRTI-binding pocket. NNRTIs that do not have the ability to wiggle and jiggle and adapt their shape to the various pockets found in the NNRTI-resistant RTs fail against the known mutants either because their binding is susceptible to steric hindrance, because they lose key hydrophobic interactions, or because mutations like K103N interfere with entry of the NNRTIs into the pocket.

### Role of the Cyanovinyl Group of TMC278

The cyanovinyl group of TMC278 is not present in the other DAPY analogs. Analysis of the crystal structures suggests that the cyanovinyl group contributes to the enhanced potency of TMC278 relative to the other DAPY analogs, and that this moiety helps TMC278 to retain potency against NNRTI-resistance mutations. As has already been discussed, the cyanovinyl group is positioned in a cylindrical tunnel connecting the NNRTI-binding pocket to the nucleic acid-binding cleft that resembles a “piston and ring” structure (FIG. 22B). The extent of the interactions between the cyanovinyl group and the hydrophobic tunnel is conserved despite rearrangements in RT and TMC278 that accompany the pocket mutations (Supplementary Table S1). Apparently, the maintenance of cyanovinyl group interactions with RT is critical for retaining the potency of TMC278 against a broad range of NNRTI-resistance mutations. Analysis of the torsional flexibility clearly demonstrates how TMC278 is resilient in overcoming the effects of drug-resistance mutations. A 2D infrared spectroscopic study of TMC278 complexed with the engineered RT52A HIV-1 RT revealed that the conformational distribution of drug-protein complexes is relaxing on the tens of picoseconds timescale; i.e., TMC278 loses structural “memory” of its binding mode within tens of picoseconds. These motions are consistent with the concept that TMC278 is flexible even when bound to HIV-1 RT and can change its conformation to adapt to the elastic NNRTI-binding pocket.

### Implications for Drug Design

High resolution structures of RT provide opportunities for understanding inhibitor-protein interactions with greater accuracy, more reliable determination of the structural effects of resistance mutations, and for systematic structure-based drug design targeting the NNRTI-binding pocket. The opening of the tunnel to the nucleic acid-binding site suggests the possibility of extending NNRTIs so that they could interact directly with the conserved residues involved in dNTP and/or nucleic acid binding, a concept that has been previously proposed. The interactions of the TMC278 cyanovinyl group with the hydrophobic tunnel enhances the binding of the inhibitor, and the group is also important for the potency of the inhibitor against drug-resistance mutations. Comparison of structures of TMC278 in complexes with L100I/K103N and wild-type RT clearly demonstrated the importance of strategic flexibility (wiggling) and repositioning (jiggling).

The RT-bound conformations of TMC278 are somewhat different from each other and from its free-state low-energy conformation obtained using the molecular modeling software Schrödinger (http://www.schrodinger.com/). However, the total energy calculated for the different conformations of TMC278 are not significantly different from its free-state low-energy conformation. It is expected that a small molecule would bind to a receptor approximately at its low-energy conformation. The fact that TMC278 can achieve near-low-energy conformations when bound to different forms of HIV-1 RT explains why TMC278 maintains its high potency against the mutant RTs.

The HIV-1 RT binding pocket for NNRTIs is flexible and can accommodate a diverse range of small molecule chemotypes. The binding pocket flexibility can be described as a “molecular shrink wrap” phenomenon in which the protein structure adapts and can form a complementary shape to surround the bound inhibitor. Analysis of the K103N/Y181C mutant RT/TMC278 structure reveals how TMC278 can take advantage of the structural flexibility of RT, inducing localized changes in the protein that lead to new interactions with Y183 that compensate the loss of the hydrophobic interaction caused by the Y181C mutation. The fact that compensatory changes can occur both in the protein and in the drug suggests that optimal drug design strategies should carefully consider and take advantage of the flexibility of both the inhibitor and protein. Considering the potential flexibility of both the protein and the drug should be strategic considerations in early stages of programs to design drugs that are intended to be broadly effective against targets that readily mutate and develop drug resistance.

This invention may be embodied in other forms or carried out in other ways without departing from the spirit or essential characteristics thereof. The present disclosure is therefore to be considered as in all respects illustrative and not restrictive, the scope of the invention being indicated by the appended Claims, and all changes which come within the meaning and range of equivalency are intended to be embraced therein.

The documents cited and listed herein, related to the above disclosure and particularly to the experimental procedures and discussions. The documents should be considered as incorporated by reference in their entirety.

We claim:

1. An isolated nucleic acid molecule encoding a peptide comprising the amino acid sequence of SEQ ID NO:1.

2. An isolated nucleic acid molecule encoding a peptide comprising the amino acid sequence of (SEQ ID NO:2).

3. The nucleic acid molecule of claim 1, further comprising SEQ ID NO: 2.

4. An isolated nucleic acid molecule encoding at least a portion of the amino acid sequence of human immunodeficiency virus reverse transcriptase (HIV-RT) wherein at least one terminal end of the protein is truncated.

5. An isolated nucleic acid molecule encoding at least a portion of the amino acid sequence of human immunodeficiency virus reverse transcriptase (HIV-RT) wherein:
a. the amino-terminus of HIV-RT p66 comprises amino acid residues MVPISP (SEQ ID NO: 4);
b. the nucleic acid molecule encodes alanine at amino acid residue 172 of p66;
c. the nucleic acid molecule encodes alanine at amino acid residue 173 of p66;
d. the nucleic acid molecule encodes serine at amino acid residue 280 of p66;
e. the nucleic acid molecule encodes serine at amino acid residue 280 of p51;
f. the carboxy-terminus of p66 terminates at residue 555; and
g. the carboxy-terminus of HIV-RT p51 terminates at residue 428.

6. The nucleic acid molecule of claim 5, wherein the amino-terminus of p51 comprises a human rhinovirus subtype 14 3C(HRV-14 3C) protease cleavage site, wherein the HRV-14 3C protease cleavage site is situated between a hexaHIS purification tag and the p51 coding sequence, thereby facilitating generation of a post-protease amino-terminus of gPISP upon exposure to HRV-14 3C protease under standard conditions for HRV-14 3C protease activity.

7. The isolated nucleic acid molecule of claim 5, wherein the nucleic acid molecule comprises the nucleic acid sequence of SEQ ID NO 3.

8. A recombinant vector comprising the nucleic acid molecule of claim 5.

9. The nucleic acid molecule of claim 5, wherein the nucleic acid molecule encodes HIV-RT p66 and the amino-terminus of p66 begins with the amino acid residues MVPISP (SEQ ID NO: 121).

10. An isolated nucleic acid molecule encoding at least a portion of the amino acid sequence of human immunodeficiency virus reverse transcriptase (HIV-RT), wherein the nucleic acid molecule encodes alanine at amino acid residue 172 of p66.

11. The isolated nucleic acid molecule of claim 10, wherein the nucleic acid molecule encodes HIV RT p66 and wherein the amino terminus of p66 comprises amino acid residues MVPISP (SEQ ID NO: 121).

12. The isolated nucleic acid molecule of claim 10, wherein the nucleic acid molecule encodes alanine at amino acid residue 173 of p66.

13. The isolated nucleic acid molecule of claim 10, wherein the nucleic acid molecule encodes encodes serine at amino acid residue 280 of p66.

14. The isolated nucleic acid molecule of claim 10, wherein the nucleic acid molecule encodes serine at amino acid residue 280 of p51.

15. The isolated nucleic acid molecule of claim 10, wherein the nucleic acid molecule encodes HIV RT p66 and wherein the carboxy-terminus of p66 terminates at residue 555.

16. The isolated nucleic acid molecule of claim 10, wherein the nucleic acid molecule encodes HIV RT p51 and wherein the amino-terminus of p51 comprises a human rhinovirus subtype 14 3C protease (HRV-14 3C) cleavage site.

17. The isolated nucleic acid molecule of claim 16, wherein the HRV-14 3C protease cleavage site is situated between a hexaHIS purification tag and the p51 coding sequence, thereby facilitating generation of a post-protease amino-terminus of gPISP upon exposure to HRV-14 3C protease under standard conditions for HRV-14 3C protease activity.

18. The isolated nucleic acid molecule of claim 10, wherein the nucleic acid molecule encodes the carboxy-terminus of p51 terminates at residue 428.

19. A composition comprising the HIV-RT product of the expression of the nucleic acid of claim 3.

20. An isolated nucleic acid or portion thereof wherein the nucleic acid:
a. encodes at least a portion of a human immunodeficiency virus (HIV) reverse transcriptase (RT); and
b. is capable of hybridizing under standard hybridization conditions to a nucleic acid sequence or complement thereof, of claim 1.

21. The isolated nucleic acid of claim 20 wherein the nucleic acid:
a. encodes at least a portion of a human immunodeficiency virus (HIV) reverse transcriptase (RT); and
b. is capable of hybridizing under standard hybridization conditions to a nucleic acid sequence or complement thereof, of claim 2.

22. The recombinant vector of claim 8, wherein the vector is a plasmid.

23. A prokaryotic host cell transformed with the vector of claim 22.

24. A eukaryotic host cell transformed with the vector of claim 22.

25. An isolated cell line comprising the nucleic acid of claim 3.

26. A method for generating crystallization variants of an HIV-RT-NNRTI complex, comprising the steps of:
a. Truncating at least one terminus of HIV-RT;
b. Reducing surface lysine acid regions; and
c. Mutating at least one amino acid residue, thereby altering lattice contact from the non-mutated residue.

27. The method of claim 26, wherein step b comprises reducing surface glutamic acid regions.

28. The method of claim 26, wherein step b comprises mutating lysine to alanine.

29. The method of claim 27, wherein step b comprises mutating glutamic acid to alanine.

30. The method of claim 26, wherein step c is systematic mutagenesis.

31. The method of claim 26, wherein step c is achieved by methylated overlap extension ligation independent cloning.

32. The method of claim 26, further comprising the step of selecting mutant HIV RT for enzymatic activity.

33. The method of claim 26, further comprising the step of crystallizing the mutant HIV-RT.

34. The method of claim 26, further comprising the step of minimizing mutation of conserved amino acid residues.

35. The method of claim 31, further comprising the step of determining the three dimensional crystal structure of the mutant HIV-RT-NNRTI complex.

36. The HIV-RT-NNRTI complex produced by the method of claim 26.

37. The method of claim 26, wherein the NNRTI is a DAPY compound.

38. The method of claim 27, wherein the DAPY compound is selected from the group consisting of TMC278 and TMC125.

39. A method for identifying HIV-RT inhibitor solvent molecules comprising the steps of
a. Soaking a small molecule fragment into a crystallization variant generated by the method of claim 26, thereby forming an HIV-RT complex with the molecule;
b. Determining three dimensional structure of the complex; and
c. Determining HIV-RT enzyme activity.


SEQUENCE LISTING (128 sequences)

SEQ ID NO: 1 - protein, 557 aa, HIV
MVPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFAAQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLSKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNKGRQKVVPLTNTTNQKTELQAIYLALQDSGLEVNIVTDSQYALGIIQAQPDKSESELVNQIIEQLIKKEKVYLAWVPAHKGIGGNEQVDKLVSAG

SEQ ID NO: 2 - protein, 443 aa, HIV
MAHHHHHHALEVLFQGPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFKKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQTYQIYQEPFKNLKTGKYARMRGAHTNDVKQLTEAVQKITTESIVIWGKTPKFKLPIQKETWETWWTEYWQATWIPEWEFVNTPPLVKLWYQ

SEQ ID NO: 3 - DNA, 6662 bp, HIV
aacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatataccatggcacatcaccaccaccatcacgctcttgaagtcctctttcagggacccattagccctattgagactgtaccagtaaaattaaagccaggaatggatggcccaaaagttaaacaatggccattgacagaagaaaaaataaaagcattagtagaaatttgtacagaaatggaaaaggaagggaaaatttcaaaaattgggcctgagaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttaaaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactacccatacaaaaggaaacatgggaaacatggtggacagagtattggcaagccacctggattcctgagtgggagtttgttaatacccctcctttagtgaaattatggtaccagtaagagctctctagagagctggctcccggggggatcccggccgcccgcgttgatctcgatcccgcgaaattaatacgactcactataggggaattgtgagcggataacaattcccctctagaaataattttgtttaactttaagaaggagatatacatatggttcccattagccctattgagactgtaccagtaaaattaaagccaggaatggatggcccaaaagttaaacaatggccattgacagaagaaaaaataaaagcattagtagaaatttgtacagaaatggaaaaggaagggaaaatttcaaaaattgggcctgagaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttgcagcacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattaagtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactacccatacaaaaggaaacatgggaaacatggtggacagagtattggcaagccacctggattcctgagtgggagtttgttaatacccctcctttagtgaaattatggtaccagttagagaaagaacccatagtaggagcagaaaccttctatgtagatggggcagctaacagggagactaaattaggaaaagcaggatatgttactaacaaaggaagacaaaaggttgtccccctaactaacacaacaaatcagaaaactgagttacaagcaatttatctagctttgcaggattcaggattagaagtaaacatagtaacagactcacaatatgcattaggaatcattcaagcacaaccagataaaagtgaatcagagttagtcaatcaaataatagagcagttaataaaaaaggaaaaggtctatctggcatgggtaccagcacacaaaggaattggaggaaatgaacaagtagataaattagtcagtgctggataactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatggtcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggc

SEQ ID NO: 4 - protein, 15 aa, HIV
MAHHHHHHALEVLFQ

SEQ ID NO: 5 - DNA, 35 bp, HIV
gcggatccggaccaaacacagaatttgcactatcc

SEQ ID NO: 6 - DNA, 77 bp, HIV
cgccatggcacatcaccaccaccatcacgctcttgaagtcctctttcagggacccattagccctattgagactgtac

SEQ ID NO: 7 - DNA, 48 bp, HIV
cggctcgagttagtttctctacaaaatattgttttttaagttgagctg

SEQ ID NO: 8 - DNA, 68 bp, HIV
gaaaagcaggatatgttactaacaaaggagcatcactgtctagccccctaactaacacaacaaatcag

SEQ ID NO: 9 - DNA, 31 bp, HIV
gctcctttgttagtaacatatcctgcttttc

SEQ ID NO: 10 - DNA, 51 bp, HIV
gacagctggactgtcaatgacatacagaagttagtggggaaattgaattgg

SEQ ID NO: 11 - DNA, 27 bp, HIV
ctgtatgtcattgacagtccagctgtc

SEQ ID NO: 12 - DNA, 37 bp, HIV
ggatccttacgccccgcccagctctctagagagctcg

SEQ ID NO: 13 - DNA, 40 bp, HIV
ccagctctctagagagctcggtgatggtgatggtgatggc

SEQ ID NO: 14 - DNA, 43 bp, HIV
ccagctctctagagagctcgttagtgatggtgatggtgatggc

SEQ ID NO: 15 - DNA, 43 bp, HIV; identical to SEQ ID NO: 14

SEQ ID NO: 16 - DNA, 39 bp, HIV
cgggagctcttactggtaccataatttcactaaaggagg

SEQ ID NO: 17 - DNA, 35 bp, HIV
gtggagctcttagaaggtttctgctcctactatgg

SEQ ID NO: 18 - DNA, 37 bp, HIV
gtggagctcttagttagctgccccatctacatagaag

SEQ ID NO: 19 - DNA, 50 bp, HIV
ctcaagacttctgggaagttcaaataccacatcccgcagggttaaaaaag

SEQ ID NO: 20 - DNA, 27 bp, HIV
gtatttgaacttcccagaagtcttgag

SEQ ID NO: 21 - DNA, 49 bp, HIV
gagctgagacaacatctgttgaggggacttaccacaccagacaaaaaac

SEQ ID NO: 22 - DNA, 26 bp, HIV
cccctcaacagatgttgtctcagctc

SEQ ID NO: 23 - DNA, 63 bp, HIV
caaaagttaaacaatggccattgacagcagaaaaaataaaagcattagtagaaatttgtacag

SEQ ID NO: 24 - DNA, 30 bp, HIV
ctgctgtcaatggccattgtttaacttttg

SEQ ID NO: 25 - DNA, 60 bp, HIV
cactaacagaagtaataccactaacagcagcagcagcgctagaactggcagaaaacagag

SEQ ID NO: 26 - DNA, 31 bp, HIV
ctgctgttagtggtattacttctgttagtgc

SEQ ID NO: 27 - DNA, 57 bp, HIV
gtagatggggcagctaacagggcgactaaattaggaaaagcaggatatgttactaac

SEQ ID NO: 28 - DNA, 24 bp, HIV
cgccctgttagctgccccatctac

SEQ ID NO: 29 - DNA, 17 bp, HIV
agccataccgcgaaagg

SEQ ID NO: 30 - DNA, 34 bp, HIV
cgggctttctccttcctctcccttatgcgactcc

SEQ ID NO: 31 - DNA, 34 bp, HIV
cgctcctcttcgggcccgccagcacatggactcg

SEQ ID NO: 32 - DNA, 37 bp, HIV
cagctggactgtcaatgacatttgtaagttagtgggg

SEQ ID NO: 33 - DNA, 21 bp, HIV
atgtcattgacagtccagctg

SEQ ID NO: 34 - DNA, 57 bp, HIV
gaataccacatcccgcagggttaaaaaagaataaatcagtaacagtactggatgtgg

SEQ ID NO: 35 - DNA, 59 bp, HIV
caaaaatcttagagccttttgcagcacaaaatccagacatagttatctatcaatacatg

SEQ ID NO: 36 - DNA, 39 bp, HIV
gattttgtgctgcaaaaggctctaagatttttgtcatgc

SEQ ID NO: 37 - DNA, 61 bp, HIV
catgacaaaaatcttagagccttttcgtaaacaaaatccagacatagttatctatcaatac

SEQ ID NO: 38 - DNA, 28 bp, HIV
acgaaaaggctctaagatttttgtcatg

SEQ ID NO: 39 - DNA, 58 bp, HIV
cttagaaatagggcagcatagaacagcaatagcggcgctgagacaacatctgttgagg

SEQ ID NO: 40 - DNA, 30 bp, HIV
attgctgttctatgctgccctatttctaag

SEQ ID NO: 41 - DNA, 48 bp, HIV
ggggacttaccacaccagacgcagcacatcagaaagaacctccattcc

SEQ ID NO: 42 - DNA, 23 bp, HIV
tgcgtctggtgtggtaagtcccc

SEQ ID NO: 43 - DNA, 48 bp, HIV
gtacagcctatagtgctgccagcagcagacagctggactgtcaatgac

SEQ ID NO: 44 - DNA, 25 bp, HIV
ctgctggcagcactataggctgtac

SEQ ID NO: 45 - DNA, 58 bp, HIV
gaaagactcctaaatttaaactacccatacaagcggcaacatgggaaacatggtggac

SEQ ID NO: 46 - DNA, 35 bp, HIV
cgcttgtatgggtagtttaaatttaggagtctttc

SEQ ID NO: 47 - DNA, 80 bp, HIV
aaaagcaggatatgttactaacagaggaagacaaaaggttgtcaccctaactgacacaacaaatcagaaaactgagttac

SEQ ID NO: 48 - DNA, 28 bp, HIV
cctctgttagtaacatatcctgcttttc

SEQ ID NO: 49 - DNA, 60 bp, HIV
ggaatcattcaagcacaaccagatcaaagtgaatcagagttagtcaatcaaataatagag

SEQ ID NO: 50 - DNA, 28 bp, HIV
tttgatctggttgtgcttgaatgattcc

SEQ ID NO: 51 - DNA, 59 bp, HIV
gtcaatcaaataatagagcagttaatagcaaaggcagcggtctatctggcatgggtacc

SEQ ID NO: 52 - DNA, 39 bp, HIV
gcctttgctattaactgctctattatttgattgactaac

SEQ ID NO: 53 - DNA, 26 bp, HIV
acgccaccatggctcaccatcatcac

SEQ ID NO: 54 - DNA, 57 bp, HIV
gaataccacatcccgcagggataaaaaagaataaatcagtaacagtactggatgtgg

SEQ ID NO: 55 - DNA, 44 bp, HIV
cttcttcgcccccgttttcacgatgggcaaatattatacgcaag

SEQ ID NO: 56 - DNA, 20 bp, HIV
tgaaaacgggggcgaagaag

SEQ ID NO: 57 - DNA, 38 bp, HIV
tagaaataattttgtttaactttaagaaggagatatac

SEQ ID NO: 58 - DNA, 20 bp, HIV
cgagctctctagagagctgg

SEQ ID NO: 59 - DNA, 20 bp, HIV
cgcgggatcgagatcaacgc

SEQ ID NO: 60 - DNA, 32 bp, HIV
gtttaactttaagaaggagatataccatggtt

SEQ ID NO: 61 - DNA, 32 bp, HIV
aaccatggtatatctccttcttaaagttaaac

SEQ ID NO: 62 - DNA, 32 bp, HIV; identical to SEQ ID NO: 61

SEQ ID NO: 63 - DNA, 44 bp, HIV
gaaactttaccagactcgagttaaagtattttcctgattccagc

SEQ ID NO: 64 - DNA, 41 bp, HIV
gaaactttaccagactcgagttagtgatggtggtggtgatg

SEQ ID NO: 65 - DNA, 30 bp, HIV
catatgtatatctccttcttaaagttaaac

SEQ ID NO: 66 - DNA, 55 bp, HIV
agaaggagatatacatatgcatatggttattgagactgtaccagtaaaattaaag

SEQ ID NO: 67 - DNA, 58 bp, HIV
agaaggagatatacatatgcatatggttcctattgagactgtaccagtaaaattaaag

SEQ ID NO: 68 - DNA, 48 bp, HIV
agaaggagatatacatatgcatatggttcccattagccctattgagac

SEQ ID NO: 69 - DNA, 32 bp, HIV
cacgcgggcggccgttggatccccccgggtcc

SEQ ID NO: 70 - DNA, 32 bp, HIV
cacgcgggcggccgtggatccttacgccccgc

SEQ ID NO: 71 - DNA, 33 bp, HIV
cggccgcccgcgtgtgttgatctcgatcccgcg

SEQ ID NO: 72 - DNA, 42 bp, HIV
gaggagaagcccggtctcgagttagtgatggtggtggtgatg

SEQ ID NO: 73 - DNA, 29 bp, HIV
gcgagctcttagtgatggtggtggtgatg

SEQ ID NO: 74 - DNA, 39 bp, HIV
cggctcgagttatccagcactgactaatttatctacttg

SEQ ID NO: 75 - DNA, 47 bp, HIV
gaggagaagcccggtctcgagttatagtattttcctgattccagcac

SEQ ID NO: 76 - DNA, 37 bp, HIV
ctccatggttattgagactgtaccagtaaaattaaag

SEQ ID NO: 77 - DNA, 54 bp, HIV
ctttaagaaggagatataccatggttattgagactggtaccagtaaaattaaag

SEQ ID NO: 78 - DNA, 40 bp, HIV
ctccatggttcctattgagactgtaccagtaaaattaaag

SEQ ID NO: 79 - DNA, 46 bp, HIV
ctttaagaaggagatataccatggttcctattgagactggtaccag

SEQ ID NO: 80 - DNA, 46 bp, HIV
ctttaagaaggagatataccatggttcccattagccctattgagac

SEQ ID NO: 81 - DNA, 30 bp, HIV
ctccatggttcccattagccctattgagac

SEQ ID NO: 82 - DNA, 39 bp, HIV
cagagctcgttacagtattttcctgattccagcactgac

SEQ ID NO: 83 - DNA, 22 bp, HIV
tagggctaatgggaaccatatg

SEQ ID NO: 84 - DNA, 48 bp, HIV
gtttctttaccagactcgagttatagtattttcctgattccagcactg

SEQ ID NO: 85 - DNA, 45 bp, HIV
cagtgctggaatcaggaaaatactacgagctctctagagagctgg

SEQ ID NO: 86 - DNA, 45 bp, HIV
ccagctctctagagagctcgtagtattttcctgattccagcactg

SEQ ID NO: 87 - DNA, 48 bp, HIV
ccagctctctagagagctcgttatagtattttcctgattccagcactg

SEQ ID NO: 88 - DNA, 30 bp, HIV
gcccccgggagccagctctctagagagctc

SEQ ID NO: 89 - DNA, 26 bp, HIV
gccggatccttacgccccgccctgcc

SEQ ID NO: 90 - DNA, 39 bp, HIV
ggcccgaagaggagcgccggtttctttaccagactcgag

SEQ ID NO: 91 - DNA, 31 bp, HIV
gaggagaaagccctgcgtcgagatcccggac

SEQ ID NO: 92 - DNA, 33 bp, HIV
gaggagaaagcccgggtatggcatgatagcgcc

SEQ ID NO: 93 - DNA, 25 bp, HIV
ccggagctctctagagagctggctc

SEQ ID NO: 94 - DNA, 20 bp, HIV
ctcaggtacacgaccgcaag

SEQ ID NO: 95 - DNA, 20 bp, HIV
gagtccatgtgctggcgttc

SEQ ID NO: 96 - DNA, 24 bp, HIV
tcctttgttagtaacatatcctgc

SEQ ID NO: 97 - DNA, 39 bp, HIV
cgggatccagcaaaaaacccctcaagacccgtttagagg

SEQ ID NO: 98 - DNA, 65 bp, HIV
ccttttaaaaaacaaaatccagacatagttatctgtcaatacatggatgatttgtatgtaggatc

SEQ ID NO: 99 - DNA, 34 bp, HIV
agataactatgtctggattttgttttttaaaagg

SEQ ID NO: 100 - DNA, 61 bp, HIV
gacatagttatctatcaatacatggatgatttgcttgtaggatctgacttagaaatagggc

SEQ ID NO: 101 - DNA, 33 bp, HIV
caaatcatccatgtattgatagataactatgtc

SEQ ID NO: 102 - DNA, 38 bp, HIV
gacgacgacaagatccatggactactgagtgagagctc

SEQ ID NO: 103 - DNA, 35 bp, HIV
cgcgggcggccgggatccccccgggtcctcctttc

SEQ ID NO: 104 - DNA, 38 bp, HIV
gaggagaagcccggtggatccccccgggtcctcctttc

SEQ ID NO: 105 - DNA, 17 bp, HIV
ttgatctcgatcccgcg

SEQ ID NO: 106 - DNA, 21 bp, HIV
gctagttattgctcagcggtg

SEQ ID NO: 107 - DNA, 20 bp, HIV
gctctcccttatgcgactcc

SEQ ID NO: 108 - DNA, 19 bp, HIV
cgggatcgagatcaacgcg

SEQ ID NO: 109 - DNA, 65 bp, HIV
ccttttgcagcacaaaatccagacatagttatctgtcaatacatggatgatttgtatgtaggatc

SEQ ID NO: 110 - DNA, 34 bp, HIV
agataactatgtctggattttgtgctgcaaaagg

SEQ ID NO: 111 - DNA, 54 bp, HIV
cggctcgagttatagtattttcctgattccagcactgactaatttatctacttg

SEQ ID NO: 112 - DNA, 59 bp, HIV
cgccatggcacatcaccaccaccatcacgctcttcccattagccctattgagactgtac

SEQ ID NO: 113 - DNA, 79 bp, HIV
cgccatggcacatcaccaccaccatcacgctcttgaagtcctctttcagggaattgagactgtaccagtaaaattaaag

SEQ ID NO: 114 - DNA, 42 bp, HIV
cggctcgagttagactaatttatctacttgttcatttcctcc

SEQ ID NO: 115 - DNA, 40 bp, HIV
cttttagaatctctgcgttttctgccagttctagctctgc

SEQ ID NO: 116 - DNA, 40 bp, HIV
gcagaaaacgcagagattctaaaagaaccagtacatggag

SEQ ID NO: 117 - DNA, 37 bp, HIV
cagctgtcttttgctggcagcactataggctgtactg

SEQ ID NO: 118 - DNA, 37 bp, HIV
ctgccagcaaaagacagctggactgtcaatgacatac

SEQ ID NO: 119 - DNA, 49 bp, HIV
ccctggtgtcgcattggctgcactaggtatggtaaatgcagtatacttc

SEQ ID NO: 120 - DNA, 47 bp, HIV
gcagccaatgcgacaccagggattagatatcagtacaatgtgcttcc

SEQ ID NO: 121 - protein, 6 aa, HIV
MVPISP

SEQ ID NO: 122 - DNA, 38 bp, HIV
  modified_base (15)..(15): gm
gcccgaagaggagcnccggtttctttaccagactcgag

SEQ ID NO: 123 - DNA, 32 bp, HIV
  modified_base (14)..(14): cm
ctcctcttcgggcncgccagcacatggactcg

SEQ ID NO: 124 - DNA, 31 bp, HIV
  modified_base (12)..(12): gm
ggagaaagcccnggtatggcatgatagcgcc

SEQ ID NO: 125 - DNA, 31 bp, HIV
  modified_base (14)..(14): um
acgcgggcggccgnggatccttacgccccgc

SEQ ID NO: 126 - DNA, 33 bp, HIV
  modified_base (14)..(14): cm
cgggctttctcctnctctcccttatgcgactcc

SEQ ID NO: 127 - DNA, 32 bp, HIV
  modified_base (15)..(15): gm
cggccgcccgcgtgnttgatctcgatcccgcg

SEQ ID NO: 128 - DNA, 31 bp, HIV
  modified_base (15)..(15): tm
cacgcgggcggccgnggatccccccgggtcc